SEQUENCE LISTING (497 sequences)

SEQ ID NO: 1 - protein, 167 aa, Escherichia coli
  REGION 1..167: note = misc_feature - TadA*7.10 (ecTadA) polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 2 - DNA, 4092 bp, synthetic construct
  misc_feature 1..4092: note = BhCas12b GGSGGS-ABE8-Xten20at P153 polynucleotide sequence
  misc_feature 1..6: note = Kozak sequence
  misc_feature 7..57: note = Sequence encoding N-terminal nuclear localization signal
  misc_feature 58..513: note = Sequence encoding BhCas12b
  misc_feature 514..531: note = Sequence encoding a linker
  misc_feature 532..1029: note = Sequence encoding TadA*8
  misc_feature 1030..1089: note = Sequence encoding Xten20 linker
  misc_feature 1090..3954: note = Sequence encoding BhCas12b
  misc_feature 3955..4002: note = Sequence encoding C-terminal NLS
  misc_feature 4003..4008: note = Sequence encoding GS linker
  misc_feature 4009..4089: note = Sequence encoding 3x hemagglutinin (HA) tag
  source 1..4092: mol_type = other DNA
gccaccatggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatcccggaggctctggaggaagctccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgacggctcttctggatctgaaacacctggcacaagcgagagcgccacccctgagagctctggctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaagaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggccagaccgtgtacatccctgagagcaaggaccagaagcagaagatcatcgaagagttcggcgagggctacttcattctgaaggacggggtgtacgaatgggtcaacgccggcaagctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 3 - protein, 1361 aa, synthetic construct
  REGION 1..1361: note = BhCas12b GGSGGS-ABE8-Xten20at P153 polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPGGSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKKGSYPYDVPDYAYPYDVPDYAYPYDVPDYA

SEQ ID NO: 4 - DNA, 4092 bp, synthetic construct
  misc_feature 1..4092: note = BhCas12b GGSGGS-ABE8-Xten20at K255 polynucleotide sequence
  misc_feature 1..6: note = Kozak sequence
  misc_feature 7..57: note = Sequence encoding N-terminal nuclear localization signal (NLS)
  misc_feature 58..822: note = Sequence encoding BhCas12b
  misc_feature 823..840: note = Sequence encoding a linker
  misc_feature 841..1338: note = Sequence encoding TadA*8
  misc_feature 1339..1398: note = Sequence encoding Xten20 linker
  misc_feature 1399..3954: note = Sequence encoding BhCas12b
  misc_feature 3955..4002: note = Sequence encoding C-terminal NLS
  misc_feature 4003..4008: note = Sequence encoding a GS linker
  misc_feature 4009..4089: note = Sequence encoding 3x hemagglutinin (HA) tag
  source 1..4092: mol_type = other DNA
gccaccatggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatccctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaaggaggctctggaggaagctccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgacggctcttctggatctgaaacacctggcacaagcgagagcgccacccctgagagctctggcgaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggccagaccgtgtacatccctgagagcaaggaccagaagcagaagatcatcgaagagttcggcgagggctacttcattctgaaggacggggtgtacgaatgggtcaacgccggcaagctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 5 - protein, 1361 aa, synthetic construct
  REGION 1..1361: note = BhCas12b GGSGGS-ABE8-Xten20at K255 polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKGGSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKKGSYPYDVPDYAYPYDVPDYAYPYDVPDYA

SEQ ID NO: 6 - DNA, 4092 bp, synthetic construct
  misc_feature 1..4092: note = BhCas12b GGSGGS-ABE8-Xten20at D306 polynucleotide sequence
  misc_feature 1..6: note = Kozak sequence
  misc_feature 7..57: note = Sequence encoding N-terminal nuclear localization signal (NLS)
  misc_feature 58..975: note = Sequence encoding BhCas12b
  misc_feature 976..993: note = Sequence encoding a linker
  misc_feature 994..1491: note = Sequence encoding TadA*8
  misc_feature 1492..1551: note = Sequence encoding Xten20 linker
  misc_feature 1552..3954: note = Sequence encoding BhCas12b
  misc_feature 3955..4002: note = Sequence encoding C-terminal NLS
  misc_feature 4003..4008: note = Sequence encoding a GS linker
  misc_feature 4009..4089: note = Sequence encoding 3x hemagglutinin (HA) tag
  source 1..4092: mol_type = other DNA
gccaccatggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatccctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaagaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacggaggctctggaggaagctccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgacggctcttctggatctgaaacacctggcacaagcgagagcgccacccctgagagctctggcgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggccagaccgtgtacatccctgagagcaaggaccagaagcagaagatcatcgaagagttcggcgagggctacttcattctgaaggacggggtgtacgaatgggtcaacgccggcaagctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 7 - protein, 1361 aa, synthetic construct
  REGION 1..1361: note = BhCas12b GGSGGS-ABE8-Xten20at D306 polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDGGSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKKGSYPYDVPDYAYPYDVPDYAYPYDVPDYA

SEQ ID NO: 8 - DNA, 4092 bp, synthetic construct
  misc_feature 1..4092: note = BhCas12b GGSGGS-ABE8-Xten20at D980 polynucleotide sequence
  misc_feature 1..6: note = Kozak sequence
  misc_feature 7..57: note = Sequence encoding N-terminal nuclear localization signal (NLS)
  misc_feature 58..2997: note = Sequence encoding BhCas12b
  misc_feature 2998..3015: note = Sequence encoding a linker
  misc_feature 3016..3513: note = Sequence encoding TadA*8
  misc_feature 3514..3573: note = Sequence encoding Xten20 linker
  misc_feature 3574..3954: note = Sequence encoding BhCas12b
  misc_feature 3955..4002: note = Sequence encoding C-terminal NLS
  misc_feature 4003..4008: note = Sequence encoding a GS linker
  misc_feature 4009..4089: note = Sequence encoding 3x hemagglutinin (HA) tag
  source 1..4092: mol_type = other DNA
gccaccatggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatccctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaagaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggaggctctggaggaagctccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgacggctcttctggatctgaaacacctggcacaagcgagagcgccacccctgagagctctggcggccagaccgtgtacatccctgagagcaaggaccagaagcagaagatcatcgaagagttcggcgagggctacttcattctgaaggacggggtgtacgaatgggtcaacgccggcaagctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 9 - protein, 1361 aa, synthetic construct
  REGION 1..1361: note = BhCas12b GGSGGS-ABE8-Xten20at D980 polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGGSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKKGSYPYDVPDYAYPYDVPDYAYPYDVPDYA

SEQ ID NO: 10 - DNA, 4092 bp, synthetic construct
  misc_feature 1..4092: note = BhCas12b GGSGGS-ABE8-Xten20at K1019 polynucleotide sequence
  misc_feature 1..6: note = Kozak sequence
  misc_feature 7..57: note = Sequence encoding N-terminal nuclear localization signal (NLS)
  misc_feature 58..3114: note = Sequence encoding BhCas12b
  misc_feature 3115..3132: note = Sequence encoding a linker
  misc_feature 3133..3630: note = Sequence encoding TadA*8
  misc_feature 3631..3690: note = Sequence encoding Xten20 linker
  misc_feature 3691..3954: note = Sequence encoding BhCas12b
  misc_feature 3955..4002: note = Sequence encoding C-terminal NLS
  misc_feature 4003..4008: note = Sequence encoding GS linker
  misc_feature 4009..4089: note = Sequence encoding 3x hemagglutinin (HA) tag
  source 1..4092: mol_type = other DNA
gccaccatggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatccctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaagaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggccagaccgtgtacatccctgagagcaaggaccagaagcagaagatcatcgaagagttcggcgagggctacttcattctgaaggacggggtgtacgaatgggtcaacgccggcaagggaggctctggaggaagctccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgacggctcttctggatctgaaacacctggcacaagcgagagcgccacccctgagagctctggcctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 11 - protein, 1361 aa, synthetic construct
  REGION 1..1361: note = BhCas12b GGSGGS-ABE8-Xten20at K1019 polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKGGSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKKGSYPYDVPDYAYPYDVPDYAYPYDVPDYA

SEQ ID NO: 12 - protein, 208 aa, Petromyzon marinus
  REGION 1..208: note = MISC_FEATURE - tr|A5H718|A5H718_PETMA Cytosine deaminase OS=Petromyzon marinusOX=7757 PE=2 SV=1 amino acid sequence; PmCDA1 polypeptide sequence
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 13 - DNA, 766 bp, Petromyzon marinus
  misc_feature 1..766: note = EF094822.1 Petromyzon marinus isolate PmCDA.21 cytosine deaminase mRNA, complete cds; PmCDA1 polynucleotide sequence
  source 1..766: mol_type = genomic DNA
tgacacgacacagccgtgtatatgaggaagggtagctggatggggggggggggaatacgttcagagaggacattagcgagcgtcttgttggtggccttgagtctagacacctgcagacatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggaattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttaagaggctatgcggatggttttc

SEQ ID NO: 14 - protein, 145 aa, Homo sapiens
  REGION 1..145: note = MISC_FEATURE - tr|Q6QJ80|Q6QJ80_HUMAN Activation-induced cytidine deaminase OS=Homo sapiens; OX = 9606 GN=AICDA PE=2 SV=1; AID polypeptide sequence
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 15 - DNA, 10681 bp, Homo sapiens
  misc_feature 1..10681: note = NG_011588.1:5001-15681 Homo sapiens activation induced cytidine deaminase (AICDA), RefSeqGene (LRG_17) on chromosome 12; nucleic acid sequence of the CDS of human AID; polynucleotide sequence
  source 1..10681: mol_type = genomic DNA
agagaaccatcattaattgaagtgagatttttctggcctgagacttgcagggaggcaagaagacactctggacaccactatggacaggtaaagaggcagtcttctcgtgggtgattgcactggccttcctctcagagcaaatctgagtaatgagactggtagctatccctttctctcatgtaactgtctgactgataagatcagcttgatcaatatgcatatatattttttgatctgtctccttttcttctattcagatcttatacgctgtcagcccaattctttctgtttcagacttctcttgatttccctctttttcatgtggcaaaagaagtagtgcgtacaatgtactgattcgtcctgagatttgtaccatggttgaaactaatttatggtaataatattaacatagcaaatctttagagactcaaatcatgaaaaggtaatagcagtactgtactaaaaacggtagtgctaattttcgtaataattttgtaaatattcaacagtaaaacaacttgaagacacactttcctagggaggcgttactgaaataatttagctatagtaagaaaatttgtaattttagaaatgccaagcattctaaattaattgcttgaaagtcactatgattgtgtccattataaggagacaaattcattcaagcaagttatttaatgttaaaggcccaattgttaggcagttaatggcacttttactattaactaatctttccatttgttcagacgtagcttaacttacctcttaggtgtgaatttggttaaggtcctcataatgtctttatgtgcagtttttgataggttattgtcatagaacttattctattcctacatttatgattactatggatgtatgagaataacacctaatccttatactttacctcaatttaactcctttataaagaacttacattacagaataaagattttttaaaaatatatttttttgtagagacagggtcttagcccagccgaggctggtctctaagtcctggcccaagcgatcctcctgcctgggcctcctaaagtgctggaattatagacatgagccatcacatccaatatacagaataaagatttttaatggaggatttaatgttcttcagaaaattttcttgaggtcagacaatgtcaaatgtctcctcagtttacactgagattttgaaaacaagtctgagctataggtccttgtgaagggtccattggaaatacttgttcaaagtaaaatggaaagcaaaggtaaaatcagcagttgaaattcagagaaagacagaaaaggagaaaagatgaaattcaacaggacagaagggaaatatattatcattaaggaggacagtatctgtagagctcattagtgatggcaaaatgacttggtcaggattatttttaacccgcttgtttctggtttgcacggctggggatgcagctagggttctgcctcagggagcacagctgtccagagcagctgtcagcctgcaagcctgaaacactccctcggtaaagtccttcctactcaggacagaaatgacgagaacagggagctggaaacaggcccctaaccagagaagggaagtaatggatcaacaaagttaactagcaggtcaggatcacgcaattcatttcactctgactggtaacatgtgacagaaacagtgtaggcttattgtattttcatgtagagtaggacccaaaaatccacccaaagtcctttatctatgccacatccttcttatctatacttccaggacactttttcttccttatgataaggctctctctctctccacacacacacacacacacacacacacacacacacacacacacacaaacacacaccccgccaaccaaggtgcatgtaaaaagatgtagattcctctgcctttctcatctacacagcccaggagggtaagttaatataagagggatttattggtaagagatgatgcttaatctgtttaacactgggcctcaaagagagaatttcttttcttctgtacttattaagcacctattatgtgttgagcttatatatacaaagggttattatatgctaatatagtaatagtaatggtggttggtactatggtaattaccataaaaattattatccttttaaaataaagctaattattattggatcttttttagtattcattttatgttttttatgtttttgattttttaaaagacaatctcaccctgttacccaggctggagtgcagtggtgcaatcatagctttctgcagtcttgaactcctgggctcaagcaatcctcctgccttggcctcccaaagtgttgggatacagtcatgagccactgcatctggcctaggatccatttagattaaaatatgcattttaaattttaaaataatatggctaatttttaccttatgtaatgtgtatactggcaataaatctagtttgctgcctaaagtttaaagtgctttccagtaagcttcatgtacgtgaggggagacatttaaagtgaaacagacagccaggtgtggtggctcacgcctgtaatcccagcactctgggaggctgaggtgggtggatcgcttgagccctggagttcaagaccagcctgagcaacatggcaaaacgctgtttctataacaaaaattagccgggcatggtggcatgtgcctgtggtcccagctactagggggctgaggcaggagaatcgttggagcccaggaggtcaaggctgcactgagcagtgcttgcgccactgcactccagcctgggtgacaggaccagaccttgcctcaaaaaaataagaagaaaaattaaaaataaatggaaacaactacaaagagctgttgtcctagatgagctacttagttaggctgatattttggtatttaacttttaaagtcagggtctgtcacctgcactacattattaaaatatcaattctcaatgtatatccacacaaagactggtacgtgaatgttcatagtacctttattcacaaaaccccaaagtagagactatccaaatatccatcaacaagtgaacaaataaacaaaatgtgctatatccatgcaatggaataccaccctgcagtacaaagaagctacttggggatgaatcccaaagtcatgacgctaaatgaaagagtcagacatgaaggaggagataatgtatgccatacgaaattctagaaaatgaaagtaacttatagttacagaaagcaaatcagggcaggcatagaggctcacacctgtaatcccagcactttgagaggccacgtgggaagattgctagaactcaggagttcaagaccagcctgggcaacacagtgaaactccattctccacaaaaatgggaaaaaaagaaagcaaatcagtggttgtcctgtggggaggggaaggactgcaaagagggaagaagctctggtggggtgagggtggtgattcaggttctgtatcctgactgtggtagcagtttggggtgtttacatccaaaaatattcgtagaattatgcatcttaaatgggtggagtttactgtatgtaaattatacctcaatgtaagaaaaaataatgtgtaagaaaactttcaattctcttgccagcaaacgttattcaaattcctgagccctttacttcgcaaattctctgcacttctgccccgtaccattaggtgacagcactagctccacaaattggataaatgcatttctggaaaagactagggacaaaatccaggcatcacttgtgctttcatatcaaccatgctgtacagcttgtgttgctgtctgcagctgcaatggggactcttgatttctttaaggaaacttgggttaccagagtatttccacaaatgctattcaaattagtgcttatgatatgcaagacactgtgctaggagccagaaaacaaagaggaggagaaatcagtcattatgtgggaacaacatagcaagatatttagatcattttgactagttaaaaaagcagcagagtacaaaatcacacatgcaatcagtataatccaaatcatgtaaatatgtgcctgtagaaagactagaggaataaacacaagaatcttaacagtcattgtcattagacactaagtctaattattattattagacactatgatatttgagatttaaaaaatctttaatattttaaaatttagagctcttctatttttccatagtattcaagtttgacaatgatcaagtattactctttctttttttttttttttttttttttttgagatggagttttggtcttgttgcccatgctggagtggaatggcatgaccatagctcactgcaacctccacctcctgggttcaagcaaagctgtcgcctcagcctcccgggtagatgggattacaggcgcccaccaccacactcggctaatgtttgtatttttagtagagatggggtttcaccatgttggccaggctggtctcaaactcctgacctcagaggatccacctgcctcagcctcccaaagtgctgggattacagatgtaggccactgcgcccggccaagtattgctcttatacattaaaaaacaggtgtgagccactgcgcccagccaggtattgctcttatacattaaaaaataggccggtgcagtggctcacgcctgtaatcccagcactttgggaagccaaggcgggcagaacacccgaggtcaggagtccaaggccagcctggccaagatggtgaaaccccgtctctattaaaaatacaaacattacctgggcatgatggtgggcgcctgtaatcccagctactcaggaggctgaggcaggaggatccgcggagcctggcagatctgcctgagcctgggaggttgaggctacagtaagccaagatcatgccagtatacttcagcctgggcgacaaagtgagaccgtaacaaaaaaaaaaaaatttaaaaaaagaaatttagatcaagatccaactgtaaaaagtggcctaaacaccacattaaagagtttggagtttattctgcaggcagaagagaaccatcagggggtcttcagcatgggaatggcatggtgcacctggtttttgtgagatcatggtggtgacagtgtggggaatgttattttggagggactggaggcagacagaccggttaaaaggccagcacaacagataaggaggaagaagatgagggcttggaccgaagcagagaagagcaaacagggaaggtacaaattcaagaaatattggggggtttgaatcaacacatttagatgattaattaaatatgaggactgaggaataagaaatgagtcaaggatggttccaggctgctaggctgcttacctgaggtggcaaagtcgggaggagtggcagtttaggacagggggcagttgaggaatattgttttgatcattttgagtttgaggtacaagttggacacttaggtaaagactggaggggaaatctgaatatacaattatgggactgaggaacaagtttattttattttttgtttcgttttcttgttgaagaacaaatttaattgtaatcccaagtcatcagcatctagaagacagtggcaggaggtgactgtcttgtgggtaagggtttggggtccttgatgagtatctctcaattggccttaaatataagcaggaaaaggagtttatgatggattccaggctcagcagggctcaggagggctcaggcagccagcagaggaagtcagagcatcttctttggtttagcccaagtaatgacttccttaaaaagctgaaggaaaatccagagtgaccagattataaactgtactcttgcattttctctccctcctctcacccacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataaggtatcaattaaagtcggctttgcaagcagtttaatggtcaactgtgagtgcttttagagccacctgctgatggtattacttccatccttttttggcatttgtgtctctatcacattcctcaaatccttttttttatttctttttccatgtccatgcacccatattagacatggcccaaaatatgtgatttaattcctccccagtaatgctgggcaccctaataccactccttccttcagtgccaagaacaactgctcccaaactgtttaccagctttcctcagcatctgaattgcctttgagattaattaagctaaaagcatttttatatgggagaatattatcagcttgtccaagcaaaaattttaaatgtgaaaaacaaattgtgtcttaagcatttttgaaaattaaggaagaagaatttgggaaaaaattaacggtggctcaattctgtcttccaaatgatttcttttccctcctactcacatgggtcgtaggccagtgaatacattcaacatggtgatccccagaaaactcagagaagcctcggctgatgattaattaaattgatctttcggctacccgagagaattacatttccaagagacttcttcaccaaaatccagatgggtttacataaacttctgcccacgggtatctcctctctcctaacacgctgtgacgtctgggcttggtggaatctcagggaagcatccgtggggtggaaggtcatcgtctggctcgttgtttgatggttatattaccatgcaattttctttgcctacatttgtattgaatacatcccaatctccttcctattcggtgacatgacacattctatttcagaaggctttgattttatcaagcactttcatttacttctcatggcagtgcctattacttctcttacaatacccatctgtctgctttaccaaaatctatttccccttttcagatcctcccaaatggtcctcataaactgtcctgcctccacctagtggtccaggtatatttccacaatgttacatcaacaggcacttctagccattttccttctcaaaaggtgcaaaaagcaacttcataaacacaaattaaatcttcggtgaggtagtgtgatgctgcttcctcccaactcagcgcacttcgtcttcctcattccacaaaaacccatagccttccttcactctgcaggactagtgctgccaagggttcagctctacctactggtgtgctcttttgagcaagttgcttagcctctctgtaacacaaggacaatagctgcaagcatccccaaagatcattgcaggagacaatgactaaggctaccagagccgcaataaaagtcagtgaattttagcgtggtcctctctgtctctccagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaaggtgcgaaagggccttccgcgcaggcgcagtgcagcagcccgcattcgggattgcgatgcggaatgaatgagttagtggggaagctcgaggggaagaagtgggcggggattctggttcacctctggagccgaaattaaagattagaagcagagaaaagagtgaatggctcagagacaaggccccgaggaaatgagaaaatggggccagggttgcttctttcccctcgatttggaacctgaactgtcttctacccccatatccccgcctttttttcctttttttttttttgaagattatttttactgctggaatacttttgtagaaaaccacgaaagaactttcaaagcctgggaagggctgcatgaaaattcagttcgtctctccagacagcttcggcgcatccttttggtaaggggcttcctcgctttttaaattttctttctttctctacagtcttttttggagtttcgtatatttcttatattttcttattgttcaatcactctcagttttcatctgatgaaaactttatttctcctccacatcagctttttcttctgctgtttcaccattcagagccctctgctaaggttccttttccctcccttttctttcttttgttgtttcacatctttaaatttctgtctctccccagggttgcgtttccttcctggtcagaattcttttctcctttttttttttttttttttttttttttaaacaaacaaacaaaaaacccaaaaaaactctttcccaatttactttcttccaacatgttacaaagccatccactcagtttagaagactctccggccccaccgacccccaacctcgttttgaagccattcactcaatttgcttctctctttctctacagcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

SEQ ID NO: 16 - protein, 198 aa, Homo sapiens [sequence omitted]

SEQ ID NO: 17 - protein, 198 aa, Canis lupus
  REGION 1..198: note = MISC_FEATURE - Canine AID (clAID) polypeptide sequence
  REGION 1..30: note = MISC_FEATURE - Nuclear localization sequence
  REGION 183..198: note = MISC_FEATURE - Nuclear export signal
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 18 - protein, 199 aa, Bos taurus
  REGION 1..199: note = MISC_FEATURE - Bovine AID (btAID) polypeptide sequence
  REGION 1..30: note = MISC_FEATURE - Nuclear localization sequence
  REGION 183..199: note = MISC_FEATURE - Nuclear export signal
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 19 - protein, 239 aa, Rattus sp.
  REGION 1..239: note = MISC_FEATURE - Rat AID polypeptide sequence
  REGION 1..26: note = MISC_FEATURE - Nuclear localization sequence
  REGION 194..239: note = MISC_FEATURE - Nuclear export signal
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 20 - protein, 198 aa, Mus musculus [sequence omitted]

SEQ ID NO: 21 - protein, 229 aa, Rattus norvegicus [sequence omitted]

SEQ ID NO: 22 - protein, 229 aa, Mesocricetus auratus
  REGION 1..229: note = MISC_FEATURE - maAPOBEC-1 polypeptide sequence
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 23 - protein, 236 aa, Pongo pygmaeus
  REGION 1..236: note = MISC_FEATURE - ppAPOBEC-1 polypeptide sequence
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 24 - protein, 236 aa, Oryctolagus cuniculus
  REGION 1..236: note = MISC_FEATURE - ocAPOBEC1 polypeptide sequence
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 25 - protein, 235 aa, Monodelphis domestica
  REGION 1..235: note = MISC_FEATURE - mdAPOBEC-1 polypeptide sequence
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 26 - protein, 224 aa, Pongo pygmaeus
  REGION 1..224: note = MISC_FEATURE - ppAPOBEC-2 polypeptide sequence
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 27 - protein, 224 aa, Bos taurus
  REGION 1..224: note = MISC_FEATURE - btAPOBEC-2 polypeptide sequence
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 28 - protein, 440 aa, Mus musculus
  REGION 1..440: note = MISC_FEATURE - mAPOBEC-3-(1) polypeptide sequence
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 29 - protein, 429 aa, Mus musculus
  REGION 1..429: note = MISC_FEATURE - APOBEC-3-(2) (Mouse APOBEC-3) polypeptide sequence
  REGION 71..108: note = MISC_FEATURE - Nucleic acid editing domain
  REGION 288..319: note = MISC_FEATURE - Nucleic acid editing domain
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 30 - protein, 430 aa, Rattus sp.
  REGION 1..430: note = MISC_FEATURE - APOBEC-3 polypeptide sequence
  REGION 72..109: note = MISC_FEATURE - Nucleic acid editing domain
  REGION 289..320: note = MISC_FEATURE - Nucleic acid editing domain
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 31 - protein, 199 aa, Homo sapiens
  REGION 1..199: note = MISC_FEATURE - hAPOBEC-3A polypeptide sequence
  REGION 70..106: note = MISC_FEATURE - Nucleic acid editing domain
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 32 - protein, 373 aa, Homo sapiens
  REGION 1..373: note = MISC_FEATURE - hAPOBEC-3F polypeptide sequence
  REGION 65..99: note = MISC_FEATURE - Nucleic acid editing domain
  REGION 248..283: note = MISC_FEATURE - Nucleic acid editing domain
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 33 - protein, 370 aa, Macaca mulatta
  REGION 1..370: note = MISC_FEATURE - Rhesus macaque APOBEC-3G polypeptide sequence
  REGION 1..61: note = MISC_FEATURE - Cytoplasmic localization signal
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 34 - protein, 384 aa, Pan troglodytes
  REGION 1..384: note = MISC_FEATURE - Chimpanzee APOBEC-3G polypeptide sequence
  REGION 1..60: note = MISC_FEATURE - Cytoplasmic localization signal
  REGION 65..100: note = MISC_FEATURE - Nucleic acid editing domain
  REGION 257..291: note = MISC_FEATURE - Nucleic acid editing domain
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 35 - protein, 377 aa, Chlorocebus sabaeus
  REGION 1..377: note = MISC_FEATURE - Green monkey APOBEC-3G polypeptide sequence
  REGION 1..60: note = MISC_FEATURE - Cytoplasmic localization signal
  REGION 65..100: note = MISC_FEATURE - Nucleic acid editing domain
  REGION 257..290: note = MISC_FEATURE - Nucleic acid editing domain
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 36 - protein, 384 aa, Homo sapiens
  REGION 1..384: note = MISC_FEATURE - Human APOBEC-3G polypeptide sequence
  REGION 1..60: note = MISC_FEATURE - Cytoplasmic localization signal
  REGION 65..100: note = MISC_FEATURE - Nucleic acid editing domain
  REGION 257..291: note = MISC_FEATURE - Nucleic acid editing domain
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 37 - protein, 382 aa, Homo sapiens
  REGION 1..382: note = MISC_FEATURE - Human APOBEC-3B polypeptide sequence
  REGION 66..100: note = MISC_FEATURE - Nucleic acid editing domain
  REGION 251..289: note = MISC_FEATURE - Nucleic acid editing domain
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 38 - protein, 395 aa, Rattus sp.
  REGION 1..395: note = MISC_FEATURE - Rat APOBEC-3B polypeptide sequence
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 39 - protein, 226 aa, Bos taurus
  REGION 1..226: note = MISC_FEATURE - Bovine APOBEC-3B polypeptide sequence
DGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 40 - protein, 490 aa, Pan troglodytes
  REGION 1..490: note = MISC_FEATURE - Chimpanzee APOBEC-3B polypeptide sequence
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 41 - protein, 190 aa, Homo sapiens
  REGION 1..190: note = MISC_FEATURE - Human APOBEC-3C polypeptide sequence
  REGION 65..100: note = MISC_FEATURE - Nucleic acid editing domain
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 42 - protein, 190 aa, Gorilla sp.
  REGION 1..190: note = MISC_FEATURE - Gorilla APOBEC-3C polypeptide sequence
  REGION 65..100: note = MISC_FEATURE - Nucleic acid editing domain
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 43 - protein, 202 aa, Macaca mulatta
  REGION 1..202: note = MISC_FEATURE - Rhesus macaque APOBEC-3A polypeptide sequence
  REGION 72..103: note = MISC_FEATURE - Nucleic acid editing domain
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 44 - protein, 185 aa, Bos taurus
  REGION 1..185: note = MISC_FEATURE - Bovine APOBEC-3A polypeptide sequence
  REGION 56..86: note = MISC_FEATURE - Nucleic acid editing domain
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 45 - protein, 200 aa, Homo sapiens
  REGION 1..200: note = MISC_FEATURE - Human APOBEC-3H polypeptide sequence
  REGION 54..88: note = MISC_FEATURE - Nucleic acid editing domain
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 46 - protein, 210 aa, Macaca mulatta
  REGION 1..210: note = MISC_FEATURE - Rhesus macaque APOBEC-3H polypeptide sequence
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 47 - protein, 386 aa, Homo sapiens
  REGION 1..386: note = MISC_FEATURE - Human APOBEC-3D polypeptide sequence
  REGION 78..109: note = MISC_FEATURE - Nucleic acid editing domain
  REGION 262..296: note = MISC_FEATURE - Nucleic acid editing domain
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 48 - protein, 236 aa, Homo sapiens
  REGION 1..236: note = MISC_FEATURE - Human APOBEC-1 polypeptide sequence
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 49 - protein, 229 aa, Mus musculus
  REGION 1..229: note = MISC_FEATURE - Mouse APOBEC-1 polypeptide sequence
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 50 - protein, 224 aa, Homo sapiens
  REGION 1..224: note = MISC_FEATURE - Human APOBEC-2 polypeptide sequence
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 51 - protein, 224 aa, Mus musculus
  REGION 1..224: note = MISC_FEATURE - Mouse APOBEC-2 polypeptide sequence
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 52 - protein, 224 aa, Rattus sp.
  REGION 1..224: note = MISC_FEATURE - Rat APOBEC-2 polypeptide sequence
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 53 - protein, 208 aa, Petromyzon marinus
  REGION 1..208: note = MISC_FEATURE - Petromyzon marinus CDA1 (pmCDAl) polypeptide sequence
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 54 - protein, 381 aa, Homo sapiens
  REGION 1..381: note = MISC_FEATURE - Human APOBEC3G D316R D317R polypeptide sequence
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 55 - protein, 182 aa, Homo sapiens
  REGION 1..182: note = MISC_FEATURE - Human APOBEC3G chain A polypeptide sequence
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 56 - protein, 184 aa, Homo sapiens
  REGION 1..184: note = MISC_FEATURE - Human APOBEC3G chain A D120R D121R polypeptide sequence
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 57 - protein, 367 aa, Homo sapiens
  REGION 1..367: note = MISC_FEATURE - hAPOBEC-4 polypeptide sequence
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 58 - protein, 388 aa, Rattus norvegicus
  REGION 1..388: note = MISC_FEATURE - rAPOBEC-4 polypeptide sequence
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 59 - protein, 363 aa, Macaca fascicularis
  REGION 1..363: note = MISC_FEATURE - mfAPOBEC-4 polypeptide sequence
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 60 - protein, 218 aa, Rattus norvegicus
  REGION 1..218: note = MISC_FEATURE - rAPOBEC-1 (delta 177-186) polypeptide sequence
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 61 - protein, 218 aa, Rattus norvegicus
  REGION 1..218: note = MISC_FEATURE - rAPOBEC-1 (delta 202-213) polypeptide sequence
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 62 - protein, 198 aa, Mus musculus
  REGION 1..198: note = MISC_FEATURE - mouse cytidine deaminase polypeptide sequence
  REGION 1..30: note = MISC_FEATURE - Nuclear localization sequence
  REGION 183..198: note = MISC_FEATURE - Nuclear export signal
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 63 - protein, 224 aa, Petromyzon marinus
  REGION 1..224: note = MISC_FEATURE - pmCDA-1 polypeptide sequence
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 64 - protein, 331 aa, Petromyzon marinus
  REGION 1..331: note = MISC_FEATURE - pmCDA-2 polypeptide sequence
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 65 - protein, 209 aa, Petromyzon marinus
  REGION 1..209: note = MISC_FEATURE - pmCDA-5 polypeptide sequence
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 66 - protein, 158 aa, Saccharomyces cerevisiae
  REGION 1..158: note = MISC_FEATURE - yCD polypeptide sequence
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 67 - protein, 194 aa, synthetic construct
  REGION 1..194: note = cytidine deaminase polypeptide sequence
MKPQIRDHRPNPMEAMYPHIFYFHFENLEKAYGRNETWLCFTVEIIKQYLPVPWKKGVFRNQVDPETHCHAEKCFLSWFCNNTLSPKKNYQVTWYTSWSPCPECAGEVAEFLAEHSNVKLTIYTARLYYFWDTDYQEGLRSLSEEGASVEIMDYEDFQYCWENFVYDDGEPFKRWKGLKYNFQSLTRRLREILQ

SEQ ID NO: 68 - protein, 192 aa, synthetic construct
  REGION 1..192: note = cytidine deaminase polypeptide sequence
MADSSEKMRGQYISRDTFEKNYKPIDGTKEAHLLCEIKWGKYGKPWLHWCQNQRMNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRLLDIFWESKCRSPNPW

SEQ ID NO: 69 - protein, 280 aa, synthetic construct
  REGION 1..280: note = cytidine deaminase polypeptide sequence
MDPQRLRQWPGPGPASRGGYGQRPRIRNPEEWFHELSPRTFSFHFRNLRFASGRNRSYICCQVEGKNCFFQGIFQNQVPPDPPCHAELCFLSWFQSWGLSPDEHYYVTWFISWSPCCECAAKVAQFLEENRNVSLSLSAARLYYFWKSESREGLRRLSDLGAQVGIMSFQDFQHCWNNFVHNLGMPFQPWKKLHKNYQRLVTELKQILREEPATYGSPQAQGKVRIGSTAAGLRHSHSHTRSEAHLRPNHSSRQHRILNPPREARARTCVLVDASWICYR

SEQ ID NO: 70 - protein, 555 aa, synthetic construct
  REGION 1..555: note = pYY-BEM3.1; tr|F7B644|F7B644_HORSE polypeptide sequence
MPRGRARERQRRNPMEKLDAEAFSFHFLNMEFVYDRNCSYLCYQVEGRLSGSPVLSEQGVFPNEVCGKTRRHAELCFLDWFRGRLSPDEYYCVTWFISWSPCSNCAREVAEFLKRHRNVELSIFAARLYYCRDHEQGLQSLCNRGAQLAVMLRKDFTYCWDNFVHNSGREFSPWENIDANSDLLARKLEDLLKNPMEKLHRKTFSFHFRNLKFAKGRKCSYLCYRVEGRLSGSPGLSEQGVFLNEVCDENCRHAELCFLHWFRGRLSPHADYRVTWFISWSPCSNCAREVAEFLKQHRNVELHISAARLYYWQRNKPGLRNLRSSGAQLAIMFFWDFRDCWDNFVHNSGRHFIPWKKINVNSRLLATKLEDLLKNPLEKLHPNTFSFHFCNLEFAYDRKYSYLCYQVEGRLSGSPGLSEQGVFLNEVCGKTRCHAELCFLDWFRVRLSPDEYYRVTWFISWSPCFYCAREVADFLKQYRNVKLSIFAARLYYCRDHAQGLRSLCSSGAQLAIMFFWDFRYCWDNFVHNSGREFRPWKKINVNSRLLATKLEDILK

SEQ ID NO: 71 - protein, 437 aa, synthetic construct
  REGION 1..437: note = pYY-BEM3.2; tr|D1LZA1|D1LZA1_PANTI polypeptide sequence
MEPWRPSPRNPMDRIDPKTFRFQFPNLRYASGRKLCYLCFQVERDYFYYNDSDWGVFRNEVHPWAPCHAEQCFLSWFRDQYPYRDEDYNVTWFLSWSPCPTCAEEVVEFLEEYRNLTLSIFTSRLYYFWHPNYQEGLCKLWDAGVQLDIMSCDEFEYCWDNFVYHKGMRFQRRNLLKDYDFLAAKLQEILSPGQQRKRDWPFPPRPGAQVDPRSWVQEVTEPGINTRRHPLHLLVSFLLPRPTMNPLQEDIFYRQFGNQHRVPKPYYYRRKTYLCYQLKLPEGTLIDKDCLRNKKKRHAEICFIDKIKSLTRDTSQRFEIICYITWSPCPFCAEELVAFVKDNPHLSLRIFASRLYVHWRWKYQQGLRHLHASGIPVAVMSLPEFEDCWRNFVDHQDRLFQPWRNLDQYSESIKRRLGKILTPLNDLRNDFRNLKLE

SEQ ID NO: 72 - protein, 440 aa, synthetic construct
  REGION 1..440: note = pYY-BEM3.3; tr|A0A3Q0DM17|A0A3Q0DM17_TARSY polypeptide sequence
MPMKRMYSNIYFDHFNNQRLLSGQNAPWLCFKVERVENCMLVPLETGVFGNQVSGCCGKTERPVEPTSLTRSVLVSPNPGTELRAQQPSRKGHLGKLGCVEYPSPGLALVMLGYGASTYCPDSSMYCPETCHHPEMCFLYWFEKTLSHEEQYQITWYVSWSPCVNCAEEVAEFLSVHPKVNLTIYAARLYCYQKLNHRQGLRRLCKEGACVKIMNYEEFDHCWENFVYNNYKSFKPWVKLQDNYELLATELDKILRIPMERMPQKKFRFHFQNLIAKDRNTTWLCFEVKNVRKKHPPDLLERGIFQNQVTPRINCHAEMCFLSWFLENMLLHGKRYQVTWYISWSPCSICAEEVAEFLSAHPKVSLTIYAARLYYFWVPGYRQGLRRLVEEGARVEIMNYEEFDYCWENFVSINNEPFQPWEGLHEKYGYLVTKLNNILG

SEQ ID NO: 73 - protein, 453 aa, synthetic construct
  REGION 1..453: note = pYY-BEM3.4; tr|A0A3Q0DNJ5|A0A3Q0DNJ5_TARSY polypeptide sequence
MEDNPEPRPRQQMDQDTFIFNFNNDPSVRGRHQTFLCYEVEHLDDDTWVPQDKYLGFLHNQPQSRSNAYCAYHAELCFLELVSSWQLDPAQRYRVTCFISWSPCSSCAQEVAAFLKKNRHVTLRILAARIYDYYQGYEDGLRTLQGVGVDITVMTSAEFGHCWNTFVDHQGSPFQPWEGLDQHSQVIWQRMQDILQVIPAKYLMEKVKYTVTVDILFKGRVPGPRYLMDQNTFTRNFINNLSVSGRRQTLLCYEVERLGGDIWVPLDQLRGFLLSQARDVLNYYQGRHAEPCFLDLVSSWQLDPAQHYRVTWFISWSPCTSCAQAVAAFLRENRHVTLRILAARIYDYHQGYEEGLRTLQRTGAHIDIMTFKEFGHCWNTFVNHKGSPFKSWTGLDQHSQALRKRLQDILHTMASSLWDQSEPKKPIPSQEVTLPESIPPSHGNRFRLVKRPS

SEQ ID NO: 74 - protein, 424 aa, synthetic construct
  REGION 1..424: note = pYY-BEM3.5; tr|G5AYU5|G5AYU5_HETGA polypeptide sequence
FCFLSCVHRKPIERIYKKAFRFYFRNLRCAYGRNKTFLCYEVKRERDNKVLHKGVVLNQVEPYMPLHAELRFLSWFHDTLLCPLGSYQVTLYVSWSPCSECAEELTTFLAGHRNVTMTIYVAQLYYCNWKSPNREGLKILIAEDARLRVMFYDEFLYCWRNFVKNDYNNFDPWSLLDENSRYHNRILQNILKGWGRPHRVGPEGEQTATPGGSGGHCISVFSLLRRREMTLKEETFRVQFNNAYKAPKPYRRRVTYLCYQLQEANGDPLTKGCLRTKKGYHAESRFIKRICSMDLGQDQSYQVTCFLTWSPCPHCAQELVSFKRAHPHLRLQIFTARLFFHWKRSYQEGLQRLCRAQVPVAVMGHPEFAYCWDNFVDHQPGPFEPPWAKLEYYSSCLKRRLQQILRSWGVDDLTNDFRNLQLGP

SEQ ID NO: 75 - protein, 385 aa, synthetic construct
  REGION 1..385: note = pYY-BEM3.6; tr|A0A2Y9QMV5|A0A2Y9QMV5_TRIMA polypeptide sequence
MLSSPQTPGTRKPMKTLAPDEFSFNFENLRLAHGRNTTFLCFQVETKAPPSLNSPDSGIFQNQDHCPSHHHAEMVFLTWFQKRLSPAQHYEVTWYMSWSPCSRCAVQVAKFLKSNSTVNLSIFVARLYYPRELETKDGLHSLWQAGAQVQIMFFQDFKYCWENFVNNEGKPFQPWKNLDENSKDWDTELKDIHRNTTDLLTEEMFYSQFYNREKKSSIPRKTYLCYQLNEPQPVKRCLHYKKGYHAVTRFIDGIVSMNLDPARSYDITCYFTWSPCNRYARKLVSFIEDYPNLRLKVYTSRLYFHWCWTNMQGLQHLQNSRVTVAVMTFRDFEYCWKNFVDNQGKPFEPWEKLDLYSQSTERRLRRILKPLTPDVLNEDFGNLHL

SEQ ID NO: 76 - protein, 381 aa, synthetic construct
  REGION 1..381: note = pYY-BEM3.7; tr|H0XHI0|H0XHI0_OTOGA polypeptide sequence
LSCAFRDPMNRMYPKTFCQNFEKEPCPSNQNSSWLCFEVETKNSAVFFHRGVFRNQPAPPPRAPTSVLLSQGPVKTPCHAEECFLTWIQGVLPPDHHYHVTWYVSRGPCANCANLIVHFLAMHRRVTLTIFAAHLNFFWESDFQQGLLRMDQEGVQLHIMGYEEFEYCWDNFVYNQRKQFVPWNGLNENYEFMVSTLEDILRSPLDRIRQKDFSIHFRNSLWLDDKSTWLCFEVKRTKSPVPLYRGVFRNQSPPKTPCHAEVRFFTWLQDLPPDFCCQFTWYLSWSPCADCADLVANFLAKHRNVSLTIFVARLYYYRDPEMHRGLRRMYQEGANVDIMSVIEFEYCWDNFVYNQGKQFVPWNGLNENYEFLVPRLQEILE

SEQ ID NO: 77 - protein, 337 aa, synthetic construct
  REGION 1..337: note = pYY-BEM3.8; tr|A0A3M0K4Y7|A0A3M0K4Y7_HIRRU polypeptide sequence
MYISKKALRRHFDPRVYPRETYLLCELQWEGSRRVWIHWIRNVPDHHAEEYFLEEVFEPRNYGFCNITLYLSWSPCCTCCSKIRDFLKRNPNVKIDIRVARLIYPDYAETRSSLRELNGLQRVSIQVMEAAGLSCIESKNHRISQVERDPKGSSSPTLFTLQDHLKLSNMTESVIQDSVSIQICYQMRILGFQCHIRWKLQPEDFQRNYSPNQIGRVVYLLYEVRWRRGSIWRNWCSNNPEQHAEVNFLENHFHHRPQTPCSITWFLSTSPCGKCSRRILEFLKSQPNVTLEIYAAKLFRHHDIRNRQGLRNLMMNGVTIYIMNLEGNPASLCLSVD

SEQ ID NO: 78 - protein, 237 aa, synthetic construct
  REGION 1..237: note = pYY-BEM3.9; tr|A0A3P4LUZ8|A0A3P4LUZ8_GULGU polypeptide sequence
MSFEDYEYCWETFVDHKGMYFQSWDLLRDNDLLAAELKNILRSTMNPLRQEIFYHQFGNQPRAPRPYHRRKTYLCYQLQPHEGPITARVCLQNKKKRHAEIRFIDNIRALRLDRSQTFEITCYLTWSPCPTCAKALAVFVQDHPHISLRLFASRLFIHWCWKYQEGLRLLHRSRIPVAVMRLQEFEDCWRNFVDNQDEPFQPWNKLEQYSESITRRLRRILGHPQNNLENDFRNLHI

SEQ ID NO: 79 - protein, 221 aa, synthetic construct
  REGION 1..221: note = pYY-BEM3.10; tr|G5BPM8|G5BPM8_HETGA polypeptide sequence
RRRIEPWQFEASFDPRQLRRETCLLSEVRWGTSPRAWRGCSLNTARHAEVSFMDRLTSEGRLRGPVRCSITWFLSWSPCGACAQAIGEFLRQHPNVSLVIYIARLFWHVDEQNRQGLRDLVTRGVRMQVMSDPEFAHCWRNFVNYSPGQEARWPQVPPVWTWLYSLELHCILLNLPPCLKISRRHHNQLTFFQLILQNCHYQAIPSPVLLASGLIHPFVTW

SEQ ID NO: 80 - protein, 211 aa, synthetic construct
  REGION 1..211: note = pYY-BEM3.11; tr|H2M862|H2M862_ORYLA polypeptide sequence
MITKLDSVLLPKKKFIYHYKNMRWARGRHETYLCFVVKRRVGPESLSFDFGHLRNRNGCHVELLFLRHLSALCPGLWGYGATGQGRVSYSITWFCSWSPCANCSFRLAQFLSQTPNLRLRIFVSRLYFCDLEDSREREGLRMLKKVGVHITVMSYKDYFYCWQTFVARKQSKFKPWDGLHQNSVRLSRKLNRILQPCETEDFRDAFKLLGL

SEQ ID NO: 81 - protein, 353 aa, synthetic construct
  REGION 1..353: note = pYY-BEM3.12; tr|H0Y0C6|H0Y0C6_OTOGA polypeptide sequence
MYLKTFYRHFNNRPYLSRRNDTWLCFEVKTTSSNSPGSFYSGVFRNQGPRYCPWHTELCFLTWVRPIVSHHHFYQITWYMSWSPCANCAWQVATFLATHENVSLTNYTVRIYYFWRQDYRQGLLRMIEEGTQVYVMSSKEFQHCWENFVDHWGTRWVTCWNRLKKNYEFLVTRLSEILSDPKERISPNTFYNQFNNTPVPRGRKDTWLCFEVKEKNSNSPGSFHRGVFQNQVFSGTSSHARRCPPDHHYEVTWYTSWSPCAHCAWHVVNFLTSNPNVSLTIFAARLYYIYRPEIQQGLRRVFQEGAKVHIMSLKEFKYCWAKLVYNSGMRFMPWYQFNFNFLFPNTTLKGDLH

SEQ ID NO: 82 - protein, 189 aa, synthetic construct
  REGION 1..189: note = pYY-BEM3.13; tr|A0A3Q2Z5X6|A0A3Q2Z5X6_HIPCM polypeptide sequence
MDVHFMNFIYHYKNMRWAKGRNETYLCFVVKRRVGPNSLTFDFGHLRNRNGCHVELLFLRYLGRRLSYSITWFCSWSPCANCSAALSQFLSRMPNLRLRIFVARLYFCDMEDSHEREGLRLLQKAGVQVTVMSYKDYYYCWQTFVDRKKSHFKAWEDLHQNSVRLSRKLNRILQPCEMDLRDAFKLLGL

SEQ ID NO: 83 - protein, 190 aa, synthetic construct
  REGION 1..190: note = pYY-BEM3.15; tr|A0A2K6NY90|A0A2K6NY90_RHIRO polypeptide sequence
MNPHIRNPMEAMYPGTFYFHFKNLWEADNRNESWLCFAVEVIKHHSTVSWKRGVFRNQVDPETHCHAEKCFLSWFCDNTLSPKKNYQVTWYTSWSPCPECAREVAKFLARHSNVMLTIYTARLYYSQYPNYQEGLRRLNEEGVPVEIMDYEDFKYCWENFVYNGDELFKPWKGLKYNFLFLDSKLQEILE

SEQ ID NO: 84 - protein, 202 aa, synthetic construct
  REGION 1..202: note = pYY-BEM3.16; tr|Q6ICH2|Q6ICH2_HUMAN polypeptide sequence
MNPQIRNPMERMYRDTFYDNFENEPILYGRSYTWLCYEVKIKRGRSNLLWDTGVFRGPVLPKRQSNHRQEVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 85 - protein, 202 aa, synthetic construct
  REGION 1..202: note = pYY-BEM3.17; tr|G8GPV1|G8GPV1_CERNE polypeptide sequence
MDGSPASRPGHVMDPGTFTSNFNNKPWVSGQRETYLCYKVERSHNDTWVLLNQHRGFLRNQAKNRLHGDYGCHAELCFLGEVPSWRLDPTQTYRVTWFISWSPCFSGGCAEQVRAFLQENTHVRLRIFAARIYDYDFLYQEALRTLRDAGAQVSIMTYEEFKHCWDTFVDHQGRPFQPWDGLDEHSQALSGRLQAILQNQGN

SEQ ID NO: 86 - protein, 181 aa, synthetic construct
  REGION 1..181: note = pYY-BEM3.18; tr|Q1WBT6|Q1WBT6_SYMSY polypeptide sequence
MALLTAKTFRLQFNNKRRVTKPYYPRKALLCYQLTPQNGSTPTRGYFKNKKKRHAEIRFINKIKSMGLDETQCYQVTCYLTWSPCPSCAWELVDFIKAHDHLNLGIFASRLYYHWCRHQQEGLRLLCGSQVPVEVMGFPEFADCWENFVDHEEPLSFNPSEMLEELDKNSRAIKRRLEKIK

SEQ ID NO: 87 - protein, 211 aa, synthetic construct
  REGION 1..211: note = pYY-BEM3.19; tr|A0A3B4CS14|A0A3B4CS14_PYGNA polypeptide sequence
MDNTNRRKFIYHYKNVRWARGRHETYLCFVVKKRNSPDSLSFDFGHLRNRNGCHVELLFLRYIEVLCPGLWGSGVDGVRVSYAVTWFCSWSPCSNCAQRLTNFLSQTPNLRLRIFVARLYFCDEEDSLEREGLRHLQRAGVQITVMTYKDFFYCWQTFVASRERCFKAWEGLRQNSVRLSRKLNRILQVFISTPVISPLITTHLGQSWAGG

SEQ ID NO: 88 - protein, 315 aa, synthetic construct
  REGION 1..315: note = pYY-BEM3.20; tr|A0A087XZI4|A0A087XZI4_POEFO polypeptide sequence
RKVSYSVTWFCSWSPCANCSIRLAQFLHQTPNLRLRIFVSRLYFCDLEDSREREGLRILKKAGVHITVMSYKDYFYCWQTFVAKSQSKFKPWDGLHQNYIRLSRKLNRILQPALDIKKFIYHYKNLRWARGRCETYLCFVVKKKLHLFMFVIVGRNRLFDLNVTMNNKSLYLIPLHLQLLFLRHLGALCPGLWGYGVTGERKVSYSVTWFCSWSPCANCSIRLAQFLHQTPNLRLRIFVSRLYFCDLEDSREREGLRILKKAGVHITVMSYKDYFYCWQTFVAKSQSKFKPWDGLHQNYIRLSRKLNRILQVQFF

SEQ ID NO: 89 - protein, 189 aa, synthetic construct
  REGION 1..189: note = pYY-BEM3.21; tr|A0A341AEK4|A0A341AEK4_9CETA polypeptide sequence
MASDRGPSAGDATSRRRIEPWEFEVSFDPRELCKETRLLYEIKWGRSQHVWRHSGKNTTNHVECNFIEKFTSERPFHRSVSCCITWFLSWSPCWECSKAIREFLNQHPRVTLFIYVARLFQHMDPQNRQGLRDLIHSGVTIQIMGPTEYDYCWRNFVNYPPGKEAHWPRYPPPLMKLYALELHCIILVP

SEQ ID NO: 90 - protein, 180 aa, synthetic construct
  REGION 1..180: note = pYY-BEM3.22; tr|E2D879|E2D879_MUSMI polypeptide sequence
RNLISRETFNFNFENLCYAKGRKNTFLCYEVTRKDCDSPVSLCHGVFKNKGSIHAEICFLYWFHDKVLKVLTPREEFKVTWYMSWSPCFECAEQVVRFLATHHNLNLTIFSSRLYNVSDPDTQQKLCRLVQEGAQVAVMDLSEFKKCWEKFVDNDGQQFRPWKRLRTNFRYQNSKLQEIL

SEQ ID NO: 91 - protein, 221 aa, synthetic construct
  REGION 1..221: note = pYY-BEM3.23; tr|A0A2K5RDN6|A0A2K5RDN6_CEBCA polypeptide sequence
MWEAQSPGLSREWGSVAISPEDPGPLHIGRFLSCAFRHPMNAMYPGIFNFHFRNLRKAYGRNETWLCFTVEGIMNRSTVSWKSGVFRNQVGSDPFCHAEMCFLSWFRHNMLSPKKDYEVTWYASWSPCPECAGQVAEFLARHGNVRLTIFTAHLYYFWNPSFRQGLRRLSQEGASVLIMGYEDFEYCWDNFVYNDGQPFKPWKRLQDNSLSLYITLQEILQ

SEQ ID NO: 92 - protein, 206 aa, synthetic construct
  REGION 1..206: note = pYY-BEM3.24; tr|A0A2K5RDN7|A0A2K5RDN7_CEBCA polypeptide sequence
MEASPASRPRPLMGPRTFTENFTNNPEVFGRHQTYLCYEVKCQGPDGTRDLMTEQRDFLCNQARNLLSGFDGRHAERCFLDRVPSWRLDPAQTYRVTCFISWSPCFSCAREVAEFLQENPHVNLRIFAARIYDCRPRYEEGLQMLQNAGAQVSIMTSEEFRHCWDTFVDHQGHPFQPWEGLDEHSQALSRRLQAILQGNRWMILSL

SEQ ID NO: 93 - protein, 184 aa, synthetic construct
  REGION 1..184: note = pYY-BEM3.25; tr|A0A1C9CJ69|A0A1C9CJ69_CERAL polypeptide sequence
NPMKAMDPHIFYFHFKNLRKAYGRNETWLCFAVEIIKQRSTVPWRTGVFRNQVDPESHCHAERCFLSWFCEDILSPNTDYRVTWYTSWSPCLDCAGEVAEFLARHSNVELAIFAARLYYFWDTHYQQGLRSLSEKGASVEIMGYEDFKYCRENFVCDDGKPFKPWKGLKTNFRFLKRRLQEILE

SEQ ID NO: 94 - protein, 197 aa, synthetic construct
  REGION 1..197: note = pYY-BEM3.26; tr|A0A2R2Z4D2|A0A2R2Z4D2_PTEAL polypeptide sequence
MHLQVWRKVTEAWREGYTLKPWSRNPMERLYHDYFYFHFYNLPTPKHRNGCYICYQVEGTKKHSRMPLLRGVFENQESLDMMLSPGEKYRVTWYISWSPCFACVDEVIKFLREHTNVELIIFAARLYHSDILQYRQGLRKLHDAGVHVAIMSYYEFKHCLNDFVFHQGRSFCPWNDLNKNSKNLSNTLEDILRNQED

SEQ ID NO: 95 - protein, 206 aa, synthetic construct
  REGION 1..206: note = pYY-BEM3.27; tr|B7T161|B7T161_SHEEP polypeptide sequence
MTEGWAGSGLPGRGDCVWTPQTRNTMNLLRETLFKQQFGNQPRVPPPYYRRKTYLCYQLKELDDLMLDKGCFRNKKQRHAEIRFIDKINSLNLNPSQSYKIICYITWSPCPNCASELVDFITRNDHLNLQIFASRLYFHWIKPFCRGLHQLQKAGISVAVMTHTEFEDCWEQFVDNQLRPFQPWDKLEQYSASIRRRLQRILTAPT

SEQ ID NO: 96 - protein, 205 aa, synthetic construct
  REGION 1..205: note = pYY-BEM3.28; tr|A0A2R2X2G4|A0A2R2X2G4_PTEAL polypeptide sequence
MAGLGQACEGCCGQMPEISYPMGRLDPKTFSFEFKNLPYAYGRKSSYLCFQVEREQHSSPVPSDWGVFKNQFCGTEPYHAELCFLNWFRAEKLSPYEHYDVTWFLSWSPCSTCAEEIAIFLSNHKNVRLNIFVSRIYYFWKPAFRQGLQELDHLGVQLDAMSFDEFKYCWENFVDNQGMPFRCWKKVHQNYKSVLRKLNEILRRR

SEQ ID NO: 97 - protein, 309 aa, synthetic construct
  REGION 1..309: note = pYY-BEM3.29; tr|G1Q1M4|G1Q1M4_MYOLU polypeptide sequence
YAELSFLDLFQSWNLDRGRQYRLTWYMSWSPYPDCAQKLVEFLGENSHVTLRIFAADIHSLCSGYEDGLRKLRDARAQLAIMTRDELQYCWVTFVDNQGQPFRPWPNLVEHIKTKKQELKDILGNPMRRMYPKTFNFNFQNLNSYGRKSTFLCFEVETWEDGSVLDYQNGVFQNQLDPGHAELCFIEWFHEKVLFPDEVRCPDAQYHVTWYISWSPCFECAEQVAGFLNEHENVDLSISAARLYLCEDEDEQGLQDLVAAGAKVAMMAPEDFEYCWDNFVYNRGWPFTYWKHVRRNYGRLQEKLDEILW

SEQ ID NO: 98 - protein, 216 aa, synthetic construct
  REGION 1..216: note = pYY-BEM3.30; tr|A0A1S3AN78|A0A1S3AN78_ERIEU polypeptide sequence
RRIEPWEFEDFFDPRQFRPETCLLYEVRWGSSRNAWRSTARNTTRHAEVNFLERFAAERHFDKPVSCSITWFLSWSPCWECSQAIGAFLSQHPQVTLAIHVTRLFHHEDEQNRQGLRDLLARGVTLQVMGDSEYAHCWRTFVNSPPGAEGHYPRYPSDFTRLYALELHCIILGLPPCLEILRRYQNQFTLFRLVPQNCHYQMIPHLNFFVVRHYFF

SEQ ID NO: 99 - protein, 201 aa, synthetic construct
  REGION 1..201: note = pYY-BEM3.32; tr|Q4VUI3|Q4VUI3_XENLA polypeptide sequence
MTMDSMLLKRNKFIYHYKNLRWARGRHETYLCYIVKRRYSSVSCALDFGYLRNRNGCHAEMLFLRYLSIWVGHDPHRNYRVTWFSSWSPCYDCAKRTLEFLKGHPNFSLRIFSARLYFCEERNAEPEGLRKLQKAGVRLSVMSYKDYFYCWNTFVETRESGFEAWDGLHENSVRLARKLRRILQPPYDMEDLREVFVLLGL

SEQ ID NO: 100 - protein, 191 aa, synthetic construct
  REGION 1..191: note = pYY-BEM3.33; tr|E2RL86|E2RL86_CANLF polypeptide sequence
MNPLQEETFYQQFSNQRVPKPTYQRRTYLCYQLKPHEGSVIAKVCLQNQEKRHAEICFIDDIKSRQLDPSQKFEITCYVTWSPCPTCAKKLIAFVNDHPHISLRLFASRLYFHWRQKYKRELRHLQKSGIPLAVMSYLEFKDCWEKFVDHKGRPFQPWNKLKQYSESIGRRLQRILQPLNNLENDFRNLRL

SEQ ID NO: 101 - protein, 226 aa, synthetic construct
  REGION 1..226: note = pYY-BEM3.34; tr|G1LWB0|G1LWB0_AILME polypeptide sequence
SSAAPASIHLLDEDTFTENFRNDDWPSRTYLCYKVEGPDQGSGVPLGQDKGILHNKPAQGPEPSRHAECYLLEQIQSWNLDPKLHYGVTCFLSWSPCAKCAQKMARFLQENSHVSLKLFASRLYTRERWDEDYKEGLRTLKRAGASIAIMTYREFEHCWKTFVLHDQEGSCFQPWPFLHKESQKFSEKLQAILQVGVLLLSLPPPLPSSPLSSPWPFPAPLRASTG

SEQ ID NO: 102 - protein, 238 aa, synthetic construct
  REGION 1..238: note = pYY-BEM3.35; tr|A0A1U7S7K7|A0A1U7S7K7_ALLSI polypeptide sequence
MGEHWQYAGSGEYIPQDQFEENFDPSVLLAETHLLSELTWGGRPYKHWYENTEHCHAEIHFLENFSSKNRSCTITWYLSWSPCAECSARIADFMQENTNVKLNIHVARLYLHDDEHTRQGLRYLMKMKRVTIQVMTIPDYTYCWNTFLEDDGEDESDDYGGYAGVHEDEDESDDDDYLPTHFAPWIMLYSLELSCILQGFAPCLKIIQGNHMSPTFQLHVQDQEQKRLLEPANPWGAD

SEQ ID NO: 103 - protein, 220 aa, synthetic construct
  REGION 1..220: note = pYY-BEM3.36; tr|A0A2R2X2J8|A0A2R2X2J8_PTEVA polypeptide sequence
MPRIGNMNLLSEKTFNYHFGNQLRVKKPQGRRRTYLCYKLKLPNETLVKGYFINKKKNHAEIRFINKIRSLNLDQTQSYKITCYITWSPCSYCAGKLVALVKSCPHLSLQIFTSRLYYHWLWKNQAGLRYLWKINISVLVMKEPEFADCWDNFVNHQSRRFKPWEKLTQYSNSTERRLLRILRINRTDLFLAQSSEQDPGLNDLVDAIKRLFLDAHRPRD

SEQ ID NO: 104 - protein, 198 aa, synthetic construct
  REGION 1..198: note = pYY-BEM3.37; tr|A0A151P6M4|A0A151P6M4_ALLMI polypeptide sequence
MAVEEEKGLLGTSQGWKIELKDFQENYMPSTWPKVTHLLYEIRWGKGSKVWRNWCSNTLTQHAEVNCLENAFGKLQFNPPVPCHITWFLSWSPCCQCCRRILQFLRAHSHITLVIKAAQLFKHMDERNRQGLRDLVQSGVHVQVMDLPDYRYCWRTFVSHPHEGEGDFWPWFFPLWITFYTLELQHILLQQHALSYNL

SEQ ID NO: 105 - protein, 158 aa, synthetic construct
  REGION 1..158: note = pYY-BEM3.38; tr|A0A2K6MNR2|A0A2K6MNR2_RHIBE polypeptide sequence
IWLCFTMEIIKQCSTVSWKRGVFRNQVDPETHCHAERCFLSWFWEDTLSPNTNYQVTWYTSWSPCLDCAGEVAEFLARHSNVKLAIFAARLYYFWDTDYQQGLRSLSEEGTSVEIMGYEDFKYCWENFVYNGDEPFKPWKGLKYNFLFLDSKLQEILE

SEQ ID NO: 106 - protein, 182 aa, synthetic construct
  REGION 1..182: note = pYY-BEM3.40; tr|F1CGT0|F1CGT0_ANOCA polypeptide sequence
KAAILLSNLFFRWQMEPEAFQRNFDPREFPECTLLLYEIHWDNNTSRNWCTNKPGLHAEENFLQIFNEKIDIKQDTPCSITWFLSWSPCYPCSQAIIKFLEAHPNVSLEIKAARLYMHQIDCNKEGLRNLGRNRVSIMNLPDYRHCWTTFVVPRGANEDYWPQDFLPAITNYSRELDSILQD

SEQ ID NO: 107 - protein, 230 aa, synthetic construct
  REGION 1..230: note = pYY-BEM3.41; tr|C7AGG3|C7AGG3_HORSE polypeptide sequence
MDPQAPTQRGGLGQAYQGGDYVQAPGNGNTQHLLSEDVFKKQFGNQRRVTKPYYRRKTYVCYQLKLLRGPTIAKGYFRNKKKRHAEIRFIDKINSLGLDQDQSYEITCYVTWSPCATCACKLIKFTRKFPNLSLRIFVSRLYYHWFRQNQQGLRQLWASSIPVVVMGYQEFADCWENFADNRGNPFQSWEKLTEYSKGIKRRLQKILEPLNLNGLEDAMGNLKLGSVDLG

SEQ ID NO: 108 - protein, 197 aa, synthetic construct
  REGION 1..197: note = pYY-BEM3.42; tr|A0A250YMK7|A0A250YMK7_CASCN polypeptide sequence
MSLLKEDIFLYQFNNQQQVQKPYFRRRTYLCYQLEQPNGSRPQWPAKGCLQNKKGHHAEIRFIKRIHSMGLEQDQDYQITCYITWSPCLACACALAELKNHFPRLTLRIFASRLYFHWIRKFQMGLQHLYKSGVLVAVMSLPEFTDCWEKFVNHRQVFFTPWDKLEEHSRSIQRRLRRILQSWDVDDLTDDFRNLRL

SEQ ID NO: 109 - protein, 189 aa, synthetic construct
  REGION 1..189: note = pYY-BEM3.43; tr|B7T160|B7T160_SHEEP polypeptide sequence
MPWISDHVARLDPETFYFQFHNLLYAYGRNCSYICYRVKTWKHRSPVSFDWGVFHNQVYAGTHCHSERRFLSWFCAKKLRPDECYHITWFMSWSPCMKCAELVAGFLGMYQNVTLSIFTARLYYFQKPQYRKGLLRLSDQGACVDIMSYQEFKYCWKKFVYSQRRPFRPWKKLKRNYQLLAAELEDILG

SEQ ID NO: 110 - protein, 541 aa, synthetic construct
  REGION 1..541: note = pYY-BEM4.1; tr|A0A182D0J1|A0A182D0J1_BLAVI polypeptide sequence
MTNPESPPQAPCDFNEDALLNREPLRGSPIKFVSPVDYPDLVFALAGPVGVDIDYIQQSISDCLKSFDYSTEFIRITEIMQDIKCSKTIDCTDMLKEYQSKIEYANELRRAYRAKDLLAALTISAISKLREQIKERDEATNKSNIQPSRRKLAWVVRQLKTPEEVRLLRAVYGKQFVLVSIYSSPQRREDFLISKIKIKSRGTIDNNTSSEGAQRLIERDSKEDNEYGQNLSGTFCLGDIFVDSNNKESAIVSIDRFLNAFFGSNEISPTRDEYGMYLAKTASLRSCDLSRQVGAAIFSKTGEIISLGSNEVPKAGGGTYWTGDNADSRDIRLGHDPNEINKVEIFAEIISRLLEDKLLSNDLLNKDAASIVTILLSKNEGKRYKDLRVMDIIEFGRIIHAEMSAICDAARNGRAIIGATLFCTTFPCHLCAKHIVASGIGRIVYLEPYPKSYAKKLHSDSIQVEDHSDSEKVSFEPFIGISPSRYRELFEGGRRKDPFGEALKWKNDPRKPVIDVVVPPHFEAEKLVIAQLGKLIVSGTG

SEQ ID NO: 111 - protein, 330 aa, synthetic construct
  REGION 1..330: note = pYY-BEM4.2; tr|A0A2D6EXD2|A0A2D6EXD2_9ARCH polypeptide sequence
MIIGLVGTIGAGKQTIIDYLQEKYGYNALSCSDVLREILKKQGKPVTRDNLREIGNKTREEGGNGAIAKILLEKLRNNWKANYIVDSLRHPDEVSVLRTSPLFHLVAVDADLRIRFERVKARKREEEPTTLPAFVERDQKEMFGTGNEQRIRETMELADELVLNNGTVEELKQRIDDLNLVSDERLRPSWDDYFMRLARLAAQRSNCMSRKVGAIITKDRRVIATGYNGTPRGVKNCNEGGCERCNSAVAKGTAISECLCLHGEENAIIEAGRVRSEGATIYTSFLPCLWCTKMIIQAGLKEVVFSEVYDLHEASIKLFETSGVLIRRLK

SEQ ID NO: 112 - protein, 367 aa, synthetic construct
  REGION 1..367: note = pYY-BEM4.3; tr|F7YVM7|F7YVM7_9THEM polypeptide sequence
MNEFKYMSLALKLAKKGKYTTSPNPMVGAVIVKDGKILATGYHKKAGQPHAEINALSKLNFQAQNCEMYVTLEPCSHYGRTPPCADAIIRSGIRKVVIATLDPNPLVNGKGVEKLKNAGIEVVCGVLEEKAKKLNEKFFKYITTKIPFVALKIAQTLDGKIALKNGESKWITSEKSREYVHKLRMEYDAVLTGIGTILKDDPQLNVRLKKVYKQPLRIILDSKLKIPLSAKVLEDPSKVIILTTALADKEKLEELRSKGVEVIITNEKNGIVDLESALKILGEKKITSVMVEAGPTLLTSFLKESLFDKIYLFIAPKIFGADSKSVFSELGLEDISKSQKFSLESVKKIGEDLLLELYPKQLKKLEE

SEQ ID NO: 113 - protein, 449 aa, synthetic construct
  REGION 1..449: note = pYY-BEM4.4; tr|A0A3M6UNF1|A0A3M6UNF1_9CNID polypeptide sequence
MEEKSELENELMRSTSPKPSVPNGSKGNECEQRETRITKENLYMVLALWMEEFPVVEQTSSAKRLNKVGVVFVLPTDRVLAADCSRDGVHGVARVMVNHCGKLEGCKVFVSRKPCSLCAKLLVQSKVSRVFYLPIEPESENKGEIARADNLFKNSSVGQSVFVPCVEQKVLDKLEDKLPKEIITPDDISECRDNLLKKCGWSAEWFARAQASLPWPCFEGKMKSQVDNDFKSLIKWIAVVKAPMDKGVAFPKVKLTSDSRVVPDCDADNFPDSKTAYHMMIFAKMLARQTDDPKTGVGAVIVRGKVPDIVSLGWNGFPSKALYGEFPRASDDDRALQKKFPYVIHAEQNALMVRNVKDLTDGILFVTKPPCDECAPMIKLSGVKTIVIGEKIEKSRGGELSYNLIKEYIKEGIMTCYQMEATKTKAKRLASDPETRKRLKSSCSNSNDV

SEQ ID NO: 114 - protein, 414 aa, synthetic construct
  REGION 1..414: note = pYY-BEM4.5; tr|A0A2G3K826|A0A2G3K826_9BURK polypeptide sequence
MTKIIDDVNTAAAAVLDQATAAANQTTFAVGGVMVNNQTGEVISAIHNNVIIPLSNNVSFTFDPTAHGERQLVYWYYANKEALKLPEPNQITVITSLDPCAMCTGALLTAGFNVGVVAIDTYAGINCAQNFQFATLPANLRTKAQKNFGYYASGAANFKPLTRSYVGGPSVAFKNGVVTPANLRDCGTVFTQSVDTVRNTSNSTGLAPSQMSNPAELPSNSAILQAYRAIYKKAFTIKIDNPRLPDAQILTELKAVLADAPNARNAVAFIDPFGNLVLCMADAFNTSPVHAAFMNVTQEYAKTRWDLMNKYAQASTTDNPALYLTHPKYGTFVYLYAPDPDDSITIMSLGAYGSTMEGPIPNMFPSNLQFYYPPRNGAQFSELVPVVNELPPFYTQNVNISLMQVPGVTQAPTK

SEQ ID NO: 115 - protein, 308 aa, synthetic construct
  REGION 1..308: note = pYY-BEM4.6; tr|K1ZCJ4|K1ZCJ4_9BACT polypeptide sequence
MSSRAKKNRSTNLKKSIGQKSIENKPTDQKKDQVLVAYVPVIHEGYRRFFRHFPAVKELWLISQELSHELRSLQKDIRALKASETKKLLQTWGQFQKIKLLTPSSLAILQKTTTQLVFPDEEISHHLVEKYFAQNRVLFASFFLRWDKKSSLKKHDLQEYSEISNKEFDQMMIAIAQQEADKSDDWWRQVGGLIFKDETILLLAHNQHTPTEAEAYFAGDPRADFHQGEYLKISTAIHAEAYLIAQAAKQGISLEGADLYVTTFPCPVCAKQVAYSGIKRVFFREGYSLLDGETILKANGVKLIRVTV

SEQ ID NO: 116 - protein, 615 aa, synthetic construct
  REGION 1..615: note = pYY-BEM4.7; tr|A0A1G3PNQ8|A0A1G3PNQ8_9SPIR polypeptide sequence
MRDLPLLVLGLTGPMGAGCTRFARDISKMEPGKVIKKQGLLDQVAHEISELSKKASEIRLQCISNGKNSELAELKRLNRRLNAKLAERACLHVIAKSSLPEPLFISLNTIVIKIAVDSITAPEFAEWAKNHAKVADLLKWLRTQWESELTLYETWGQDAGRFSQDELEKMDAMFAEFERIGDEILKEDFETYFGKRNNDFSIRMFSENIRLSGNPFRPAENGGGGGKYDEPSMVMIARETDRYIRFYRTRSDQKRSHFFIIDEIKNPREAEYFRARHQNFFLVSIFSSSEIRASRMRRGLGHDAGVSDADFQHLFRELDSRDWGADDFDAHGLHRQNIYRCFNLADIAINNDVEDERFSEVLFNKFIRYYALMLSPGCVQPTPQETYMHLAYSLSLRSTCISRQVGAVITDLEDRILSLGWNEVPEGQIGCGLKVKKDYTDKENPLFEMEIWDNVITAEDLAVWDDEDSICVKDILSRIEIKTKLKSVSLTPEERADVLKALRIKRLEYSRSLHAEENAILQVASRGGVGLKDGTIYVTTFPCELCSKKIYQVGISKIYYTEPYPNSISEKVILKDGIRNIKILQFEGVKSYSYFKLFKPGFDKKDAQMLEGRGI

SEQ ID NO: 117 - protein, 291 aa, synthetic construct
  REGION 1..291: note = pYY-BEM4.8; tr|A0A1G0PGF4|A0A1G0PGF4_9BACT polypeptide sequence
MKHNNQLRKEIEKLLGQNSIIKNDELKKLQKEYKIETDELLISFLPYAAEFAKVPISKYKVGAVVLGKSGNIYFGSNMEFEAGALSATVHAEQSAVNNAWLNGETGINKIAVTAAPCGYCRQFLNELTTAKQLHVLLKDKNLEAAKVFKLTELLPEAFGPRDLEIEGGLMKVENHKLKIENINDELINAALEAANKSYAPYSKNYSGVSIQLSDGTIFSGRYSENAAYNPSLLPFQSALAFMNMNTKKGSNNKIVDAVLVEAVSNISQKDAAGTLLNSISKTKLRYYKIKN

SEQ ID NO: 118 - protein, 398 aa, synthetic construct
  REGION 1..398: note = pYY-BEM4.9; tr|A0A0P4WGY5|A0A0P4WGY5_9EUCA polypeptide sequence
MEENSSATSQPKCASRTKQGGNDLSTDMSNLSVGETKRTDFLPWDDYFMAVAFLSAMRSKDPSSQVGACIVNADKKIVGIGYNGMPIGCSDDELPWNKESLDPLQTKYMYVCHAEMNAIMNKNSSDLAGCCVYVALFPCNECAKLVIQAGIREVVFFSDKHQQKPETVASKKMLNMAGVAYRQYTPSQSKIELNLSLKEQEKSEPTADITQSSERDQNSKRKDYLSWEEYFMAMAHLSALRSKDPITQVGACIVNSKKKIVGIGYNGMPLGCNDDLMPWGNSSSNKLETKYMYVCHAGVNAIMNKNSCDVSGCTLYVALFPCNECAKVIIQAGIKTIIYASDTNKDQASILASKKMLDMAGIKYRADNLSQRKIVIDFKTIDWNSRFMNDHQNDPTCL

SEQ ID NO: 119 - protein, 259 aa, synthetic construct
  REGION 1..259: note = pYY-BEM4.10; tr|A0A3D8IG27|A0A3D8IG27_9HELI polypeptide sequence
MRKNILYFILTLFFLSGLYATSLPEDNVVSGVIYEKIDTVSAEVDHIYPMLALAIVYKDWQEKNMLNKQGHNIGLVIVDENNMPVFWVRNSVHATHNGTQHGEVRLVSNLLNCEGFNKYLDKYTLYTTLEPCIMCAGMLSMVQIPKVVYAQKDLSCGNTQEIISTAKYPRYYKAFTVENGYKKDLEECFEQYKICKNDSITDFLVNDSAKEIFRKASNDLQDYKVKFKENRRVIKVAQEFLQNIQTKDNLDVLQCPKNM

SEQ ID NO: 120 - protein, 256 aa, synthetic construct
  REGION 1..256: note = pYY-BEM4.11; tr|A0A351C8C4|A0A351C8C4_9BACT polypeptide sequence
MNELTKQSEHLRNEALRIATRSYVPYTGQQEGVIILLENGDLIPGVRVENASFQLTIPALQNALSTMYALQRTDISMIVSSIPFTDSDLAYTGGMAEIAWEMVGASLLLVAGAHIPEAGTFIDPARGENLLDVSREAALNAFIPESDFPVGSAIQTSDDVVIDGCNVEHSDWSKIICAERNVLSTARSYGLGQITTIYVSCPKEPGGTPCGACRQVIVELAPDATVWMDRGNQEPIAMKATKLLPGHFTGNVLKKQ

SEQ ID NO: 121 - protein, 301 aa, synthetic construct
  REGION 1..301: note = pYY-BEM4.12; tr|A0A1G6V2K7|A0A1G6V2K7_PEPNI polypeptide sequence
MPIVRVNEIGARLPEDWEALETAIWQAYVSREDLPDAGELDLTLVDDATIQELNKTHRQLDKSTDVLSFPMYDDRDDLAADVQAGLPVILGDIMISVPTAERQAQAYGHSFKREMAYLLVHGLLHIAGYDHMSAEEKSAMRRAEEAILADVDVPRDTAPSKTAAVLDEADVQALIDAARAARLQAYAPYSGYAVGAALLAADGRRFCGVNVENASYGATCCAERTALFAAVTAGARDFIALALVTEGDEPAPPCGLCRQALAEFSPDLAIYLAGPTGETYRRTSLAALFPEAFSLSTKESV

SEQ ID NO: 122 - protein, 528 aa, synthetic construct
  REGION 1..528: note = pYY-BEM4.13; tr|F2NP91|F2NP91_MARHT polypeptide sequence
MPVMETHALEARFKEALARLCPEGRLLAAVSGGGDSVALLYLLKAAGRDTIVAHLDHALRPDSAADAAFVEKLAQRLGFPLETEHVDVRALAHRKRINLEAAAREVRYAFLARVARRWKARCILTAHTLDDNAETVLLQILRGAGRGLGIRPLQRRVARPLLEFSRAELRAYLEARGARWLEDPTNRSLELDRNYLRHAVLPRITARFPHALEALARFSQAQQADDWALEALSARHLIPDRRWPVPAYRALPLERAPEALRRRAIRGVLEALGVRPEARLVADVEAALGGRAQTLPGGVVVRRQRGTLFFIPPTVRFPKVQPPAGLEARPPRPGDYLVFPYGRKRLVDFLNERGVPRELKRRWPVGAVGAEVRWVYGLWPEPDEDRYMRRALVLARAAARQGEVPIGAVLVRDGAVLAEAANAVEASRDATAHAELLALRTALRRVGEKVLPGATLYVTLEPCPMCYGAILEARVARVVYGVENLKAGAFTVHGLEPRVALEAGRVEGECAKVLKDFFARLRPGRDGA

SEQ ID NO: 123 - protein, 226 aa, synthetic construct
  REGION 1..226: note = pYY-BEM4.14; tr|A0A316TX77|A0A316TX77_9BACT polypeptide sequence
MINGYTPYSGNQNTCYVKGESGTFYPGVRIENVSYPLTISSVQAAVCSCLANSDNPVEYYTGDHQPELLQVWADEYDMKPGGKLPDSPLKLFDPLVPSIPDIKKELDVLTEKSVTPNSGFPVSALLQTEKGYIRGVNIELSSWALGLCAERVAISRALTAGYTQFKSIHIYAPEADFVSPCGACRQVLLEVMPDADTELYHGDGTLSKHIVSDLLPFGFTSHKLKK

SEQ ID NO: 124 - protein, 321 aa, synthetic construct
  REGION 1..321: note = pYY-BEM4.15; tr|R6VYG3|R6VYG3_9FIRM polypeptide sequence
MIHKGTQTIETKRLILRAFTPDDAEAAFENWMSDPKVTEFLRWKTHADISDSRKIVNEWANGSADPEFYQWAIVPKDVNEPIGTISVVDRNDALGIFHIGYCIGSKWWHKGITSEAFSAVIHFLFEEVGANRIESQHDPENIHSGDVMKKCGLTFEGTLRQADFNNRGIVDACVYSILQSEWQNNTSVWQRLYNAALTVQNDRVVSPFIDAGGVAAALMTKKGNIYTGICIDTASTLGMCAERNAVANMLTNGESRIDKIVAVMPDGKVGAPCGACREYMMQLDRDSGDIEILLDLETEKTVRLKDLIPDWWGAERFGDTE

SEQ ID NO: 125 - protein, 229 aa, synthetic construct
  REGION 1..229: note = pYY-BEM4.16; tr|A0A3C1HZ18|A0A3C1HZ18_9BACI polypeptide sequence
MGDIMENWNELSEPWKRCFLQAWKAYCHGSIPIGAVLVDSEGEIFLEGRNRVHELTAPEGQLCDCRIAHAEMNVLVQVKTSDYEKLSGATIYSTMEPCIQCFGAIILSRIKNISFAAIDDKLAGATTLEDRHGFIKSRNLNIAGPFSHLGEIQIILRTDFLLRIFDSEYADPLIAAHEKDYPIGVALGRHYHRNNRLQVAKKETIPFGELFNEFSFDIKRAREGYTLGK

SEQ ID NO: 126 - protein, 205 aa, synthetic construct
  REGION 1..205: note = pYY-BEM4.17; tr|A0A1M6KV24|A0A1M6KV24_9BACT polypeptide sequence
MEASQQNILLKIEGKGPVAEINFTVTLPEWLVEQVQSGSTVFLTQKEKMRFVLELARKNVAQETGGPFAAAVFSLESGELVSAGVNVVVESRCSSAHAEVVALSLAQKAVDSHDLGAAGLPRMVLVSSAEPCAMCMGAIPWSGVKQVICGARDEDVRSVGFDEGAKPLEWVEDFAERGIEVIRDVLREEATEVLWDYRERGGEIY

SEQ ID NO: 127 - protein, 207 aa, synthetic construct
  REGION 1..207: note = pYY-BEM4.18; tr|A0A2U0T9B4|A0A2U0T9B4_9RHIZ polypeptide sequence
METAELISRLLDVIEKDIAPVTAKGVARGNKLFGAAILKKSDLAVIVAETNNEIENPLWHGEMQAIKRFFELPADQRPATRDCLFLATHEPCSLCLSGITWSGFDNFYYLFSHQDSRDGFAIPYDIQILKSVYAVPEPETGTVSPARDLYNRSNDFWTSHGLQDMIAGLARSNREALLARIDDLNALYAELSERYQRDKGGKGIPLP

SEQ ID NO: 128 - protein, 231 aa, synthetic construct
  REGION 1..231: note = pYY-BEM4.19; tr|A0A2K9PN08|A0A2K9PN08_9FLAO polypeptide sequence
MSDKKESKIKISKTSESIELDEIHSLLSYSIVQKFWENDDRNGRGYNVGVILVDENKNIVDWDINSVNKTENSTQHGEMRLISRYLDKDELYSLKGYTMYPTLEPCAMCAGMMTMTNVYRTVNGQMDYFYSKALERLSIDTRECGGYPPYPRTVISEISPSSISTRLDAEYKQYTNAGNKPIITKFLSTYKAKTIYDDAFNQFINFKCKFPENKTKYENAIKFYNSLPESI

SEQ ID NO: 129 - protein, 330 aa, synthetic construct
  REGION 1..330: note = pYY-BEM4.20; tr|F4PWM7|F4PWM7_CAVFA polypeptide sequence
MRFSLSLLFVILSVLLAGVLACKDPYNPETVDYGQCASATKANYEVRSDSKVLTPADLPADELAVHESRMRHIIDIARVNNKKFVSSIYFPNGTLACIGINTGKPNMIAHGEIVAIQNCTEIHGISMYTNYSIYTTGEPCSMCASAILWSRFKTVVWSTYNSDLYCKICMSNIPIDSSYIFSRAYGLGIEAPVAIGGVVKAEGDAWFGTYCNRPTSIYYIAPKCACQDPAKVSPLKFTQTRTTVWVEGGDKVVTQWNAIISNPSNSTIVDPPIVISPSVVFKGAPWGISAASEPNTYKLSYNKVLFPGQTFSFGYSVYGLEEVAFTALEA

SEQ ID NO: 130 - protein, 193 aa, synthetic construct
  REGION 1..193: note = pYY-BEM4.21; tr|U7QZM1|U7QZM1_PHOTE polypeptide sequence
MNKTRRKLLATLGIMSISMSFIAQAGEKKTQVINNILSKQEITEHEKYMREAIKEAIKNPKHPFGAVIVNRNNGEILSRGVNTGRNNPILHGEIQAINHYITQYGNQGWENVALYTTGEPCSMCMSALVWIGIREVIWATSISVIRNSGIRQIDISAHEIAERASSFYNPITLVGGILANETDKLFLERKRGN

SEQ ID NO: 131 - protein, 319 aa, synthetic construct
  REGION 1..319: note = pYY-BEM4.22; tr|A0A081CH48|A0A081CH48_PSEA2 polypeptide sequence
MASRRHLLATQVTGNHRKLSLWHLRGWLSPYTKLVDAVYFLTTNSFYHSLQTPPVQSITMLLSSIITSLALAAQASAYREGLHPEFQSGLSINSVPATDRDHWMRLANSAIYYPPVSHPCPQAPFGTAIVNTTSNELICAIANRVGSTGDPTQHGEITAIQHCTNVMRKKGLSPQEIIAAWKQLSLYTNAEPCTMCLSAIRWAGFKEVIYGTSVGTISENGRNQIYIPSNLVLEKSYSFGHATLMLGNILTHETDPFFQHQFNESAPCPVGCERTQVGEARVKTCEPVPNWQKLVRLEYSEDSRVGSEPVAHTPLHLEL

SEQ ID NO: 132 - protein, 203 aa, synthetic construct
  REGION 1..203: note = pYY-BEM4.23; tr|A0A3D3HMU1|A0A3D3HMU1_9GAMM polypeptide sequence
MDYSDAILGAITSIRRNSKQPGVNVTDNVTDSSTQYNNDEYWMRRALALAREAGEAGEIPVGAVLVKDNQQVAGGFNQPIRSHDPAAHAEILTLREAGAVLGNYRLIDTTLYVTLEPCMMCAGALVHSRIKRLVFGAAEPKTGAAGSFIDLLTLPRLNHYMEVTGGVLGEECSVLLSDFFRRRRAEKKALKRQNSESGSDSAS

SEQ ID NO: 133 - protein, 270 aa, synthetic construct
  REGION 1..270: note = pYY-BEM4.24; tr|A0A1N5WT13|A0A1N5WT13_9ACTN polypeptide sequence
MLEKIERRLVAAAEAVVRSPSTGDAHTVAAAAMDANGDIYSGVNVFHFTGGPCAELVVIGSAAAANAPPLITIVAVGDGDRGVIAPCGRCRQVMLDLHPDVFVIVPTGDGQLAAKPVRELLPFGYVARTGSTAPRVVYFHPRHYDTISSGLKTATVRFQDSVQTGPAVFVFDDGESIRRLDAVVEKVESRRLDHLTEEDAHHEALPDSDALRDAIKTQYPMLGDGDVVDVATFRLTAISAPDPDPRSSYPPAVSRCNPAGPRADLLVGQS

SEQ ID NO: 134 - protein, 257 aa, synthetic construct
  REGION 1..257: note = pYY-BEM4.25; tr|X0SAC5|X0SAC5_9ZZZZ polypeptide sequence
MTKDGRVIASAHDTEVTDQDSTAHAEINAIRKASKIYRKDLTGCLIISTHEPCPMCTGSIIWSNISKVVYGVSIRDSIKAGRDMINLSCKEIIKKPNAEINIYDGILKKECLKLYNNDTRKLVKKFRKYEWINIEENLLNKRMQWFENNKTMIRKLKGNDLEKAYHLILMKIGIKRSEAPIVKKSESKIIFHSKNYCPSLEACIILDLDTREVCKEIYERPTEELIRRLNSKLRFTRNYDCIRPYSDYCEEIIILEK

SEQ ID NO: 135 - protein, 154 aa, synthetic construct
  REGION 1..154: note = pYY-BEM4.26; tr|A0A3B8IC10|A0A3B8IC10_9BACT polypeptide sequence
MPSHEDFIHQCLELGKEALLQGNPPVGSVIVWQDQVIGRGIENGRSSGDITQHAELLALQEAVATGQRDKLKEAIIYSTHEPCVMCAYPIRQYKIPTVVYSVAVPELGGHTSSWHLLTTEDVPKWGKAPKIITGISAEEVEALNAAFQDSLKKG

SEQ ID NO: 136 - protein, 180 aa, synthetic construct
  REGION 1..180: note = pYY-BEM4.27; tr|A0A2N9P8B9|A0A2N9P8B9_9FLAO polypeptide sequence
MFIFKLISPPVSIEVYQDKIIQKLYICFMENIFTDEYFMKKALQEAETAFQQGEIPVGAVIVIDNRIIARSHNLTEMLNDVTAHAEMQAITASANFLGGKYLKDCTLYVTLEPCQMCAGALYWSQISKIVYGATDEQRGYRAMGAQLHPKTKVISGIMQNECTHLMKDFFKQRRSKSTKD

SEQ ID NO: 137 - protein, 178 aa, synthetic construct
  REGION 1..178: note = pYY-BEM4.28; tr|K1KX30|K1KX30_9BACT polypeptide sequence
MVKNPVNNNELYFGKHSEIPMNEEQKAYMKMAVDLSRSGMESGKGGPFGCVIVKDGKVIGIGSNSVLETNDPTAHAEIVAIRDACRNLGHFQLDGCEVYTSCEPCPMCLGAIYWARPSKVFFANDKRDAAEAGFDDDFIYQELELPYEKRKIPFEQGMQDTAKEVFQEWILKEDKTLY

SEQ ID NO: 138 - protein, 196 aa, synthetic construct
  REGION 1..196: note = pYY-BEM4.29; tr|R4XI84|R4XI84_TAPDE polypeptide sequence
MSSEIEPPSTDVHKHAVAEAADESGAADAFMQIALQQAETALLNKEVPVGCVFVHQPTGTVLATGANQTNASLNGTLHAEFVAIESILRDHPPSIFRESDLYVTVEPCVMCASALRQLQVRKVYFGCGNDRFGGCGSVFSIHSDASKTGDAAYMVESGIFRKEAIMLLRRFYLLQNESAPKPALKSTRVLKEHFDE

SEQ ID NO: 139 - protein, 212 aa, synthetic construct
  REGION 1..212: note = pYY-BEM4.30; tr|A0A239CVF7|A0A239CVF7_9DELT polypeptide sequence
MSPASKKHFPSLFSFLLLTIGLICGTAHAQPQGHTADDTAATLANASLKEHEPFIRRCYQLAIDAGKKGNHPFGALLVHKGKIVLEAENTVLTDNDFTNHAEMNLIAEAARTLSRQIIPEATVYTSCAPCAMCTATLAMAGFTRIVYGVSHDALNKRFGLKGKSVSCPALFKTMGMELEFVGPVLEKEGLRVFDFWPEKDPHAQMLKKQARK

SEQ ID NO: 140 - protein, 320 aa, synthetic construct
  REGION 1..320: note = pYY-BEM4.31; tr|A0A1Q3NME1|A0A1Q3NME1_9BACT polypeptide sequence
MTEFNYDWAKLAFSSKRPLTNLKATFIIAPREISEKRFTQLLKEYLPKGDILLGISKEDYVEGLEGQPQFAMLQQKTLQKLIDKVNDASAHKVYTLRYFQRELPAIIEKLTPPRVVGIHGSWHHSFHTLPIYYLLSEKRIPYQLVAAFSDEDEARAYEVATDKKIVRPTLEGSFDDTTVLQLTDEVAKSSYDYGFQTGAILAEKVNGVYQPVAAGFNKVVPYQTYALLNGASRETNFSPANDMNHYDTIHAEMQILVEAAKQGISLKDKTLFVNLMPCPSCARTLSQTELSEIVYRIDHSGGYAVDLLTKVGKDIRRIVY

SEQ ID NO: 141 - protein, 180 aa, synthetic construct
  REGION 1..180: note = pYY-BEM4.32; tr|A0A2G6N4N7|A0A2G6N4N7_9DELT polypeptide sequence
MKERTVSYSDRHFMAEALEMAESALTQGEFPVGCVIADGTAVVARGHRTGTTAGAVNEIDHAEINALRHLGLAGEHLDRTDLTIYSTMEPCLMCFAAIVLSGINRIVYAYEDVMGGGTGCDLTGLPPLYRDAPLTLVAGVRRRASLNLFRRFFTDPENGYWAGSLLSRYTLNQTKDSHRL

SEQ ID NO: 142 - protein, 156 aa, synthetic construct
  REGION 1..156: note = pYY-BEM4.33; tr|A0A0G0RBB8|A0A0G0RBB8_9BACT polypeptide sequence
MQSVQYNKLTHLQRRALDEAEQVLENSYNPYSHFYVGACLISEDEQLIAGTNFENAAYGSAICAERAAVLRANAMSIRRFRGIAIIARGEDFNTTEVTGPCGSCRQVLYEISQVSGCDLQVILATSKKDKIVITTIRELLPLAFGPLDLGVDIGKY

SEQ ID NO: 143 - protein, 217 aa, synthetic construct
  REGION 1..217: note = pYY-BEM4.34; tr|A0A327L2Q5|A0A327L2Q5_9RHIZ polypeptide sequence
MVTSRDGEDEAMMARCVALSRIAVGKGEYPFGAVVAREGRIVAEAINRTIRDGDVSRHAEVIALARAQKAIGRRELRECSLYSNVEPCAMCSYCIREAWVGRVVYALGSPVMGGVSKWNILRDDGLSGRMPQVFDAAPEVVSGVLVEQAQAAWRDWSPLAWEMITLRGLMTDPSARPECRTRAARPRSLWHHLVALIERPPRPYVDPTSAAEGHADL

SEQ ID NO: 144 - protein, 162 aa, synthetic construct
  REGION 1..162: note = pYY-BEM4.35; tr|S2DR30|S2DR30_9BACT polypeptide sequence
MKMKKKIEITVSLEVIQKSEWSKEDRSLIERAIHAVEHAHAPYSNFMVGTALLLDNGQIFSANNQENVSFPVGICAERAVLSYAMGNFPNNRPVKLAVVAKRRSDSTWATVTPCGLCRQTINEYEVKFGHPIEILMLNPGEEILKASGIDQLLPFRFNDLNS

SEQ ID NO: 145 - protein, 153 aa, synthetic construct
  REGION 1..153: note = pYY-BEM4.36; tr|A0A369QGF1|A0A369QGF1_9BACT polypeptide sequence
MEEHEKWMHWCLNLAQQALQQGDFPVGAVVVQKGKLIGQGVEAGQLKKDITCHAEMEAIRDARQTINTADLQNCILYSTHEPCIMCSYVIRHHKISRVVVGTTVPEVGGSSSAYPLLSAPDISIWVAPPHLVTGVLAEACQALSQAYKQKFKK

SEQ ID NO: 146 - protein, 159 aa, synthetic construct
  REGION 1..159: note = pYY-BEM4.37; tr|A0A1W6X4U4|A0A1W6X4U4_9RHIZ polypeptide sequence
MTNPSRQERWDRRFLELAKVFGTWSKDRSAGTGCVIVGPDRLLRASGYNGFARGIDDEVPERHERPAKYSWTEHAERNAIYNAAKLGISLDGCTAYVNWFPCIDCARAIVQAGIVRLVGLHPDHADQRWGSEFKFATEMLRESGIEIILYDIPELAARK

SEQ ID NO: 147 - protein, 490 aa, synthetic construct
  REGION 1..490: note = pYY-BEM4.38; tr|A0A238BW09|A0A238BW09_9BILA polypeptide sequence
MEEMARKIRTKAKKANSYCNTMTFLISKASIVLLKAECKRIELTVVIFRFLIKMNASEPNNELCDMTVIKSMLKITHVIFDLDGLLIDTEVVFSKVNQCLLSKYNKKFTPHLRGLVTGMPKKAAVTYILEHEKLSAKVDVDEYCKKYDEMAEEMLPKCSLMPGVMKLVRHLKTHSIPMAICTGATKKEFEIKTRYHKELLDLISLRVLSGDDPAVKRGKPAPDPFLVTMDRFKQKPEKAENVLVFEDAANGVCAAIAAGMNVIMVPDLTYMKIPEGLQNKINSFSDNLIISNDLNVALMSLKKELSEEEVHFLNRAFEIAVDAVLNNEVPVGCVFVFEGQEVAFGRNDVNRTKNPTYHAEMVALKMMKQWCMDNGRDLEEIMRRTTLYVTLEPCIMCASALYHLRLKKILYGAANERFGGLVSVGTREKYGAKHFIEIMPNLSVDRAVKLLKEFYEKQNPFCPEEKRKVKKPKKSGNNNDNSDDAVALNV

SEQ ID NO: 148 - protein, 159 aa, synthetic construct
  REGION 1..159: note = pYY-BEM4.39; tr|A0A1J5H6Z0|A0A1J5H6Z0_9BACT polypeptide sequence
MAYQPSEKFMQMAIDKTREGVLSGQTPFGACIVKDGKVVACEHNTVWQDTDITSHGEVHTIRAACKAIGSIDLSGCILYSTCEPCPMCFSAIHWARIDTVVYGAFIADAQDAGFNELTISNEKMKEFGGSPVNFISGFMRDENVALFKLWKEQGANNVY

SEQ ID NO: 149 - protein, 168 aa, synthetic construct
  REGION 1..168: note = pYY-BEM4.40; tr|A0A3C2D945|A0A3C2D945_9BACT polypeptide sequence
MKTTEIRIIVHEYQNIDELTENDQYLLHEARRITEFAYAPYSGFHVGAAILLGNGMIVKGNNQENSAYPSGLCAERVALFYANANYPDSEVKTIAISAAKNGILVNDPIKPCGGCRQTLSEAEVRFGSPIRIILDGQDSILVLHGVESLLPLSFSKKDLASPLAATGR

SEQ ID NO: 150 - protein, 214 aa, synthetic construct
  REGION 1..214: note = pYY-BEM4.41; tr|A0A1I7EYS3|A0A1I7EYS3_9BURK polypeptide sequence
MKFKLDPSRPPDEDDYYLGVALAVRRKANCTGNRVAAVIVKNKRVIATGYNGVPEDMPNCLDGGCLRCSNPGGQFKSGTRYDLCICVHAEQNALLTAARFGISVEGAHLYTTMQPCFGCAKEILQAKIEKVFYLHPWVPTDVDPVMDAAMKAEYAKIIGKLKVKKLDFDDPVATWAVTTMRQAALASDKNPDKKTPPKTAKKKVAKKKSRTSPR

SEQ ID NO: 151 - protein, 155 aa, synthetic construct
  REGION 1..155: note = pYY-BEM4.42; tr|H8GQX8|H8GQX8_METAL polypeptide sequence
MNHEHFMRRAIELARQAPQYPFGAVIVRRDDGQCVGQGFNRSDLNPTYHGEMVAINDCAVRHCAEDWRGFDLYTTAEPCAMCQGAIEWAGIGRVFYGTSIPYLQKLGWWQIDLRAAEVSARAVFRDTLIVGGILETECNALFAAARRGCFGTGSE

SEQ ID NO: 152 - protein, 156 aa, synthetic construct
  REGION 1..156: note = pYY-BEM4.43; tr|A0A0S8HZN3|A0A0S8HZN3_9CHLR polypeptide sequence
MDEHDIRFLRASFDVARNARKNGNHPFGALLVDEHGRIVMEAENTVITAKDCTGHAETNLMREASSKYDSDFLANCTIYTSTEPCPMCAGAIFWSNVRRVVYGLSEESLYEIAGRGSEEVLFLSCREIFERGKKLIEVIGPLLEDEAREVHMGFWR

SEQ ID NO: 153 - protein, 193 aa, synthetic construct
  REGION 1..193: note = pYY-BEM4.44; tr|E3SF31|E3SF31_9CAUD polypeptide sequence
MKPTTVLQIAYLVSQESKCCSWKVGAVIEKNGRIISTGYNGSPAGGVNCCEHAEEQGWLLNKPKPVLIPGHKSECVRFSQVDRFVLAKAHREAHSAWSKNNEIHAELNAILFAARMGSSIEGATMYVTLSPCPDCAKAISQSGIKKLVYCETYDKNIPGWDDILKNAGIEVFNVPKRSLDKLNWENINEFCGE

SEQ ID NO: 154 - protein, 330 aa, synthetic construct
  REGION 1..330: note = pYY-BEM4.45; tr|F8AAC6|F8AAC6_THEID polypeptide sequence
MIRAPWHEYFMLLAKIVALRSGCNSRPSGAVIVKNKRILATGYNGPMPGAWHCTDRGPGYCFRREKGIPDIDKYNFCRATHAEANAIAQAARFGISVEGASLYCTLAPCYVCLKLIASAGIKKVYYEHDYGSRDFERDQFWKEAIKEAGLEKFEQITVSQEVMEQLQEILPYPTSKRRLAPTEFLDEFEDGKKYGVPSIEVLFNKLNYLTRQALKDITFVIEKTTVTEEPEGISFYLSGKMVELSELINTVKKQINADQNFYFLAKHNAIEAKIEILREAENIRLKAFLNECPLESFKRIAESLDYILYQVSNSLSLPTRLELSVNLLRI

SEQ ID NO: 155 - protein, 158 aa, synthetic construct
  REGION 1..158: note = pYY-BEM4.46; tr|A0A2H4ZNK4|A0A2H4ZNK4_9EUKA polypeptide sequence
MKKQLSRKIQEEWMSRLLRNAYDAGTYGEVPIAAVILNESGQCIGWGRNCREKDQNPLGHAEIIALRQASYLKKSWRFNECTMLVTLEPCPMCAGALLQARINHIIYGASDYKRGGFGGVLDLSKNSSAHHKIEITRGVKSIQSCQLLETWFRRRRRV

SEQ ID NO: 156 - protein, 176 aa, synthetic construct
  REGION 1..176: note = pYY-BEM4.47; tr|A0A239N5N1|A0A239N5N1_9PSED polypeptide sequence
MEGRAGIIPFDEGGAAMGPAEEDSPMQHLAYMREALALARANVEAGGRPFGAVLVRDGEVIARAANGTHLDHDPTAHAELLALRAAGRALGSPRLDGCVVYASGHPCPMCLAAMHLSGVSAAYYAYSNADGEPYGLSTAAVYAQMAQPVEWQSLPLQALRPEDEEGLYGFWRERRP

SEQ ID NO: 157 - protein, 199 aa, synthetic construct
  REGION 1..199: note = pYY-BEM4.48; tr|A0A328VTR2|A0A328VTR2_9PSED polypeptide sequence
MHPEHLALLQQAPASTHADDTWARLCCEQALLAVEEGCYAVGALLVDGAGELLCSGRNQVFAPAYASAAHAEMRVLDQLEAEHAQVDRRSLTLYVSLEPCLMCYGRILLAGITRVRYLARDRDGGFALRHGRLPPAWANLASGLSVVQAKADPYWLDLAEHAIGRLQDRQTLRQRVIRAWRGQRTLTDEFSSTKRTHSG

SEQ ID NO: 158 - protein, 402 aa, synthetic construct
  REGION 1..402: note = pYY-BEM4.49; tr|A0A103YG48|A0A103YG48_CYNCS polypeptide sequence
YIRELHASSLRRDEHEIQNPKILVIVDRLSSPSLHVSLSLSLSLVIFPPFIPLNQTPTHMENAKVVEAKDGTIAVASAFSGHQEVVQDRDHKFLTRAVEEAYKGVECGDGGPFGAVVVHKDEVVASCHNMVLKHTDPTAHAEVTAIREACKKLNKIELSDCEIYASCEPCPMCFGAIHLSRIKRLIYGAKAEAAIAIGFDDFIADALRGTGFYQKAHLEIKQADGNGAMIAEQVFEKTKAKFAIDHKFLTRAVEEAYKGVECGDGRPFGALVVHKDEVVVSCHNMVLNYTDPTAHAEITAIREACKKLNRIELSDCEMYSSCEPCPMCFGAIQISRIKRLVYGAKAEASIASGIPIGDFISDALKGTGFHEKANFEIKQADGNGAMIAEQVFERTKAMFPKR

SEQ ID NO: 159 - protein, 195 aa, synthetic construct
  REGION 1..195: note = pYY-BEM4.50; tr|W5M1M8|W5M1M8_LEPOC polypeptide sequence
NSSTRESRVMAQMEINGGASPPKKPGKGQSAADQDMITGLINKALQAKEFAYCPYSNFRVGAALMTNDGRVFTGCNVENACYNLGVCAERTAILKAVSEGYESFRAIAVSSDLQDQFISPCGACRQVMREFGTGWDVFLTKVDGSYVRMTVDELLPMSFGPDDLKKKKVFSLQNGHEVSTQFYTHSPCEAGENNN

SEQ ID NO: 160 - protein, 134 aa, synthetic construct
  REGION 1..134: note = pYY-BEM4.51; tr|A0A3N5YPZ2|A0A3N5YPZ2_9ALTE polypeptide sequence
MSNSETEHIQALVDAAQAAQKQSYSPYSSFQVGAAIFADDGNTYSGCNIENVAYPLGQCAEATAIGMMIMQGAKRIEDIMIASPNDQVCPPCGGCRQKISEFGTAETKIHMVTRSGEVSTVTLGELLPLAFDSL

SEQ ID NO: 161 - protein, 162 aa, synthetic construct
  REGION 1..162: note = pYY-BEM4.52; tr|A0A2A9NC86|A0A2A9NC86_9AGAR polypeptide sequence
MTNSTLSNEDRTRLIQGAFQARKKTYSPYSNFPVGAALLTTDGRIIEGANIENASYGGTICAERTAIVKAVSDGYRHFAGIAVTTKMPTRVSPCGICRQVLREFCSLDMPVLLVPGDYPQRNPVDDDGADKPGVITEGGVRETTLGALLPDSFGPENLPPRA

SEQ ID NO: 162 - protein, 163 aa, synthetic construct
  REGION 1..163: note = pYY-BEM4.53; tr|A0A2D6RD43|A0A2D6RD43_9GAMM polypeptide sequence
MNIENLITENDETLIRRCIELAGESVKNGDKPFGALLAKDGNIIFESSNNAKTKVPYHAEILTLMDAQDKLNTTDLSDYALYSNCEPCPMCSFMIREYKLDKVVFSVHSPYMGGQSRWNILEDDVLTRFKPYFSKPPNVVGGVLESEGKRIFDKVGLWMFGKE

SEQ ID NO: 163 - protein, 204 aa, synthetic construct
  REGION 1..204: note = pYY-BEM4.54; tr|A0A0H3AVL6|A0A0H3AVL6_BRUO2 polypeptide sequence
MHAKGYSQQERRIIPFANRFRFRELCSNKSLHGLRAKFPEQYTKWDPMRKAASITKANSATPMDIALEEAHAAGERGEVPIGAVIVRDGEIIARAGNRTREFNDVTAHAEILTIRQAGEMLGSERLIDCDLYVTLEPCAMCAAAISFARIRRLYYGASDPKGGGIEHGGRFYTQPTCHHAPEIYPGFCEADARKILKDFFREKR

SEQ ID NO: 164 - protein, 165 aa, synthetic construct
  REGION 1..165: note = pYY-BEM4.55; tr|A0A242H531|A0A242H531_9ENTE polypeptide sequence
MFIVKNNIEVIQQQAELDAKFMKQALKLAKDASNNGNEPFGAVLVKNDKVILTGENQIHTESDPTYHAELGIIRDFCTSQKITDLSEYTLYTSCEPCCMCAGAMVWSNLDRMVYGLGHDELAEIAGFNIMIGSEEIFSKSPNRPEVAKGVLKEAAVPVYVDYFQR

SEQ ID NO: 165 - protein, 181 aa, synthetic construct
  REGION 1..181: note = pYY-BEM4.56; tr|A0A2R6XZE2|A0A2R6XZE2_9BACL polypeptide sequence
MSGRISWHEYFMAQAKLIALRATCTRLMVGAVIVRDRRVIAGGYNGSIAGDEHCIDVGCKVRDGHCIRTIHAEQNALMQCAKFGVSTDGAELYVTHFPCLNCTKLLIQAGIRHIYYEVPYRVDPYAIELLEKAGVGTTQITVDLNAYVQVMSKVSTDPALTYVPESKAQKDEYGQSVGKIV

SEQ ID NO: 166 - protein, 173 aa, synthetic construct
  REGION 1..173: note = pYY-BEM4.57; tr|A0A139SHT6|A0A139SHT6_9BACT polypeptide sequence
MSEANASSESLPSRNSPVELIAEAAGKFGRRPTWDEYFMATAVLISTRSSCERLNVGCVIVTAGESHKNRIVAAGYNGHLPGSPHTSRMRDGHEQATVHAEQNAISDAARRGSSVEGCTAYVTHYPCINCAKILASAGIAKICYRLDYHNDPLVKPMLAEAGIEIVQLGEAAS

SEQ ID NO: 167 - protein, 170 aa, synthetic construct
  REGION 1..170: note = pYY-BEM4.58; tr|A0A261DBH2|A0A261DBH2_9RICK polypeptide sequence
MVMKKKLITVKRSTEFNNFFMEEALKQAQFALDKNEIPVGAIIVNRITNKVIAKAHNIVEQTKNPVLHAEIVAINQSCQILSSKNLSDCDMYVTLEPCVMCSGAISFARIGRLFYAANDPKQGAIENGGRFFNSKSCFYRPEIYSGFSAKISENLIKEFFYNVRYQKCNP

SEQ ID NO: 168 - protein, 146 aa, synthetic construct
  REGION 1..146: note = pYY-BEM4.59; tr|A0A2N0XZK6|A0A2N0XZK6_9VIBR polypeptide sequence
MTDNSLHESYMRQAFELSKSALPGCRPNPPVGCVFVKDGEVVSSGFSQPPGNHHAEAGAIAAYTGSYDGLVAYVTLEPCSFQGRTPSCAKALVRVRPEKVYVAILDPDTRNSGAGIKILEDAGIDVEVGLLGEEVASFLNPYLIRN

SEQ ID NO: 169 - protein, 159 aa, synthetic construct
  REGION 1..159: note = pYY-BEM4.60; tr|A0A1V5R0F9|A0A1V5R0F9_9BACT polypeptide sequence
MTKKETTKLHALDDFCMKKALLLAKRAFRADEVPVGALVVDSSNKVIGRGYNQVEKRKSQRAHAEQLAIEQACKKIGDWRLEGCTLYVTLEPCTMCMGLIKLSRIERVVFGAASPLFGYQLDKNRKSQLYKKGVIKIRKGVGKATAAALLKDFFKNKRM

SEQ ID NO: 170 - protein, 167 aa, synthetic construct
  REGION 1..167: note = pYY-BEM4.61; tr|A0A2W0H8Y3|A0A2W0H8Y3_9BACI polypeptide sequence
MKNNGRLDHEYFMTEALQEAKEAGQRGDLPIGAVIVHNGRIIARGSNMRKTAGIKISHAENNAMHNCAPYLMKHASECVIYTTLEPCIMCLTTLVMANIDSIVFAADDKYMNMKPFIDANSYIRDRIHQYKGGVCRGESEALLRKYSPYAAELALNGTHPHHRKGGA

SEQ ID NO: 171 - protein, 165 aa, synthetic construct
  REGION 1..165: note = pYY-BEM4.62; tr|A0A261BDB7|A0A261BDB7_CAERE polypeptide sequence
LYKLYIFRMTTTKANLTQFEQELVDKAVGAMEKAYCKYSGFKVGAALVCEDGEIIIGANHENASYGATICAERSAMVTALTKGHRKFKLLAVATELEAPCSPCGICRQYLIEFGDYKVILGSSTSDQIIETTTYGLLPYAFTPKSLDDHEKEAEERNHQEGEKKH

SEQ ID NO: 172 - protein, 162 aa, synthetic construct
  REGION 1..162: note = pYY-BEM4.63; tr|A0A2E1PHI6|A0A2E1PHI6_9GAMM polypeptide sequence
MKELLIHSWLMLNSNSKLIMERVIELSEINLKNGKIPIAAVIVDKKNYEIISESQNEDSPIGHAELLAITKALKKLNTNRLDSTNLFVTIEPCPMCAYAISKCHINRLYFGSEDEKGGGVINGPRIFESHNLKKIDYVSHCYHEKTTQLMQSFFQLKRNQQL

SEQ ID NO: 173 - protein, 133 aa, synthetic construct
  REGION 1..133: note = pYY-BEM4.64; tr|A0A378LUA7|A0A378LUA7_9GAMM polypeptide sequence
MDTIIKKMISNAHNTLAHSYSPYSKFSVASCICTDKDNFYTGVNVENSAYGLAICAETSAISAMVTAGEKRIKSMVVMAGTNILCSPCGACRQRIYEFSTPDTLIHLCDKNSILRTFKINELLPEAFKFDFNP

SEQ ID NO: 174 - protein, 370 aa, synthetic construct
  REGION 1..370: note = pYY-BEM4.65; tr|A0A139HQ78|A0A139HQ78_9PEZI polypeptide sequence
MADSLKSKPGHARHDTALIHGLSQSDVQKLSESCVDAKSKAYCPYSHFRVGCAVLLANGDVVQGANVENAAYPVGTCAERVALGTAVGAKKGDFRALAVSTDISPPASPCGMCRQFIREFCELNTPILMYDKDGKSVVMTLEQLLPMSFGPDKLLPPGQLENGLMQTQTQSSFVTRAFSTTSSRRQDDTPQVPQSHYDFFPQTFPQGPPPKTSFSPDLKQLRKEFLQLQAKAHPDLAPQDQKRRAEALSMRINEAYKTLQSPLRRAQYLLSQQGIDVEDETAKLDDSSLLMEVMEAREAVEEVEDEEQLNEIRAENNGRIEESVRVLEDAFRDNEFEKAAQEAIRLRYWVNIEESIQGWEKGNGGGILHH

SEQ ID NO: 175 - protein, 165 aa, synthetic construct
  REGION 1..165: note = pYY-BEM4.66; tr|A0A2A9FXV0|A0A2A9FXV0_9VIBR polypeptide sequence
MCNLKENKDMDKYFHFACDATIEGMREGTGGPFGATLTRNGEVVCSVANTVLKDMDISGHAEMVAVREACKKLDTLDLSDCVMYATCEPCPMCVSVMLWAGIKTCYYASTHLDAAKHGFSDQQLRDYLDGSDTSTLNMVHIEDNRDDCAKIWTEFRHLNETKNDG

SEQ ID NO: 176 - protein, 509 aa, synthetic construct
  REGION 1..509: note = pYY-BEM4.67; tr|A0A1A8AG96|A0A1A8AG96_NOTFU polypeptide sequence
MEHSDRWSRAEPGLSTSSRETRDGSTQTDCKLQGHGPRLSKVNLFTLLSLWMELFPQEQDEENGQSQIRRSGLVVVREGKVVGLHCSGADLHAGQAAILQHGASLANCQLFFSRRPCATCLKMIINAGVRQITFWPGDPEISMLTSNQTHSQRTSQSITEASLDATAVEKLKSNSRPQICVLMQPLAPGVLQFVDETSRRSDFMERMMDDDPELDSEKLFNSDRLRHLKDFCRHFLIQTDQRHKDILSQMGLKNFCVEPYFSNLRSNMTELVEVLAAVAAGMPQQHYGFYREESLSLDPHPVDVSQAVARHCIVQARLLSYRTEDPKVGVGAVIWAKGQSACCCGTGRLYLIGCGYNAYPAGSKYAEYPQMDNKQEDRERRKYRYIVHAEQNALTFRTRDIKPDECSMLFVTKCPCDECIPLIRGAGVKHIYTSDQDRDKDKGDISYLRFGSLKGVCKFIWQRSPPVSSASSLHLTNGCVGKHVRQAEQQIYKNKKLCTKGSSGSSDIC

SEQ ID NO: 177 - protein, 148 aa, synthetic construct
  REGION 1..148: note = pYY-BEM4.68; tr|A0A3E2VN88|A0A3E2VN88_9FIRM polypeptide sequence
MEKEITNMDKQKLIQMAVDGLGRSYAPYSHFHVSAALLCADGTVYTGNNIENAAYTPSVCAERCAIFKAVGDGRREFEAIAVCGGPDGVIEDYCPPCGVCRQVMREFCDPSSFRVLVAKTAEDYREYTLEQLLPDGFGPDHLTGSGER

SEQ ID NO: 178 - protein, 221 aa, synthetic construct
  REGION 1..221: note = pYY-BEM4.69; tr|A0A2D5ZRJ2|A0A2D5ZRJ2_9BACT polypeptide sequence
MARPVHLHTGERRTEEGATESRAVAAVATAITRAPRAPPRPATGRERDGPPPRRVFGGGLRVGDPSGYDRGESKPIGGPLTEKRSDWHSYFMRIAGEVATRATCDRKHVGAVIVRNRTILSTGYNGSIRGMPHCDDVGHDMVDGHCIATIHAEANAILQAARNGVMIQDGSIYITASPCWNCFKLVANAGLKRVYYGEFYRDKRSFEVARRLGIDLMHIEV

SEQ ID NO: 179 - protein, 155 aa, synthetic construct
  REGION 1..155: note = pYY-BEM4.70; tr|A0A1B8WPS3|A0A1B8WPS3_9BACI polypeptide sequence
MEGVQLIYQFQWGNLIMTVNKEDLYLIDVARNTIKTLYVDGKHHVGAAVRTKTGKIYSAVHLEANIGRVSVCAEAIALGKAISEGESEFDTIVAVRHPDPTQENQKIEVVSPCGICRELISDYGKGTNVILKNKEGYIKTVISDLLPNKYIREDN

SEQ ID NO: 180 - protein, 156 aa, synthetic construct
  REGION 1..156: note = pYY-BEM4.71; tr|A0A1W5ZQK9|A0A1W5ZQK9_9BACI polypeptide sequence
MNRFMERAVSLAAENVRVGGQPFGAVLVKDDELVAEGVNEMHLNYDVSGHAELLAIRRAQGELQTHDLSGYTMYASGEPCPMCLSAMYFAGIKDVFYCATVEEAAQVGLEKSKNVYDDLQKSKGERSLVMKQMPLEDDQEDPMKLWDERTNHNGTS

SEQ ID NO: 181 - protein, 340 aa, synthetic construct
  REGION 1..340: note = pYY-BEM4.72; tr|A0A378V0W4|A0A378V0W4_MYCFO polypeptide sequence
MVHAQFDPTARQALAATAVEAKTRKDLTWQQIADAAELSPAFVTAAVLGQHALPARSAEAVAALLGLDDDAALLLQTIPIRGSIPGGIPTDPTIYRFYEMLQVYGTTLKALVHEQFGDGIISAINFKLDVRKVADPEGGERAVITLDGKYLPPNPFDRVRYRGGLMDFAQRTIDIARQNVAEGGRPFATVIVKNGEILAESPNLVAQTHDPTAHAEILAIRKACTRIGTEHLIGATIYVLAQPCPMCLGSLYYCSPDEVVFLTTRDAYEPHYVDDRKYFELNMFYDEFAKPWDQRRLPMRYEPRDAAVDVYKLWQERNGGERRVPGAPTSTRPGKNPRGE

SEQ ID NO: 182 - protein, 157 aa, synthetic construct
  REGION 1..157: note = pYY-BEM4.73; tr|I3XF03|I3XF03_RHIFR polypeptide sequence
MKQRCMSPKSAQRFWDNDMHNNKDRPMSENELFVAAREAMAKAHAPYSKFPVGAAIRAEDGQIYTGANIENLSFPEGWCAETTAISHMVMAGQRKIMEVAVIAEKLALCPPCGGCRQRLAEFSGASTRIYLCDETGIKKSLALSDLLPHSFETEILG

SEQ ID NO: 183 - protein, 595 aa, synthetic construct
  REGION 1..595: note = pYY-BEM4.74; tr|F8IEF3|F8IEF3_ALIAT polypeptide sequence
MDAKELETRGWLCMRAVDVIDKKRRGEALAEEELRFLIEGYVAGRIPDYQMSAFLMAVVWRGMTREETLVLTRLLADSGERLDLSGIPGVKVDKHSTGGVGDKATLVVLPLVASIGVPVIKMSGRGLGHTGGTIDKLESIPGFRTDLSVAELVAQVRQVGIALGGQTADLAPADKKLYALRDVTGTVESLPLIASSVMSKKLAGGADAIVLDVKVGDGAFMKSRSDARRLARLMVEIGEAAGRRTVAVLSNMDQPLGCAIGNALEVAEAIRVLSGEGPFDLAEIALALAEEMTVLAGVAATREEARRMLRQSVAEGRALETLRRWIAAQGGDPAVVDDPSRLPQAPVQMPYLPKKAGFVAKLSALAFGLAAMRLGAGRETKEEAIDPSVGIVLHAKVGDRVQTHRPMFTVHARTGEDALRCIQELEAAIQISDDPVEAPPLILARIDRSEALPYADLMDAAREARDRAYVPYSGFAVGAALELADGRMVTGANVENASYGLTNCAERSAVFRAVAEGGPGTKPEIRAVAVIADSPEPVSPCGACRQVLAEFCSPDTPVYLGNLQGDVRETTVGALLPGAFTDAQMANVRRQDKEA

SEQ ID NO: 184 - protein, 153 aa, synthetic construct
  REGION 1..153: note = pYY-BEM4.75; tr|A0A1G3M638|A0A1G3M638_9SPIR polypeptide sequence
MKTTNINALDKWDLRFLQMAEHVAEWSKDPSTKVGAVIVRPDRTIASVGFNGFARGVRDTVERLWNRELKYPLTVHAELNAILSAHEPVRGHSLYVSPLSPCSNCAGVIIQSGIARVVAKCGQVNNPAQWSESFNLALTAFAEAGVSVILVEH

SEQ ID NO: 185 - protein, 177 aa, synthetic construct
  REGION 1..177: note = pYY-BEM4.76; tr|A0A3D9LFR2|A0A3D9LFR2_9MICC polypeptide sequence
MEQNDHGSSGAFSDPFEDDIPLTASLPRITGTGSGIDWQRLESTARAAMTRAYVPYSRFPVGAAALVEDGRVVAGCNIENASLGLTLCAECSLVSNLQMSGGGRIVAFYCVDGNGEVLMPCGRCRQLLYEFHAPGMRLMGPDGELTMDEVLPLAFGPADMTHLSDSAASTDDPGRTR

SEQ ID NO: 186 - protein, 172 aa, synthetic construct
  REGION 1..172: note = pYY-BEM4.77; tr|A0A3B9YGB5|A0A3B9YGB5_9BACT polypeptide sequence
MAKPISKKYRKLIETAKAARKKAYSPYSRYQVGAAVLTESGRIYSGANMENASYGLCMCAERVAIANAVTRGEKVLQAVCVVGKKARPCGACRQVMLEFSTKETELLMVDIDPNARRDTVIRTRVYSMLPNPFDPFESGMLPQHPQNLLRRRKSPQPRRKRRSRPVHREVSR

SEQ ID NO: 187 - protein, 391 aa, synthetic construct
  REGION 1..391: note = pYY-BEM4.78; tr|A0A182F569|A0A182F569_ANOAL polypeptide sequence
MPRPSQFRVSSSQSLSNSQIQASQSSDSVVDITSYVNAVVKALLNLSCTKTIIKRADLVNIALKGNGRLIGRVLQDANIELKEIYGYELIEVEKSKTMILCSTLAAGSMDELNDANRRRYTFLYLILGYIFMKNGSVPETIVWEFLETLGIEEQQEHNYFGDVRKLYDSLFKQAYLTRTKQALEGLNDDVMLISWGVRSKHEVSKKDILAGFCKVMNRDPVDFKAQYIEANEKDDKMNNNINGTVDGRNTVEYSSLDASVKELIEAAIKVRNNAYCPYSNFAVGAALRTVGGDIVTGCNVENGTFGPSVCAERTAVCKAVSEGHREFTAVAVVAFQETEFTAPCGTCRQTLSEFSRKDIPIYLVKPSPVRVMVTSLFQLLPHAFSPSFLNK

SEQ ID NO: 188 - protein, 151 aa, synthetic construct
  REGION 1..151: note = pYY-BEM4.79; tr|A0A264Z0D4|A0A264Z0D4_9BACI polypeptide sequence
MEPKKLIEEAIVASKQAYVQYSNFHVGAALLTKDGKLYHGCNIENASYGLTNCAERTAIFKAVSEGEKEFQAIAVVGDTEGPISPCGACRQVLAEFFSPDTVVILANLKGDHVVTNINELLPGFFSSKDLQKKVKNCFEKNALGSSCLRPI

SEQ ID NO: 189 - protein, 229 aa, synthetic construct
  REGION 1..229: note = pYY-BEM4.80; tr|A0A1L9Q1R3|A0A1L9Q1R3_ASPVE polypeptide sequence
MPLSAEEAALVETATATINSIPLSEDYSVASAAKASDGRVFTGVNVYHFTGGPCAELVVLGVAAAAGAAQLTHIVAVANEQRGILSPCGRCRQVLLDLQPNIQVIVGKEGSEQSVPVAQLLPFSYRQPDQHTPVIFKALTSSGPVVVDFFATWCGPCKAVAPVVGKLSETYTDVRFIQVDVDKARSISQEHDIRAMPTFVLYKDGKLLDKRVVGGNMKELEEQIKAIIA

SEQ ID NO: 190 - protein, 18 aa, synthetic construct
  REGION 1..18: note = synthetic peptide
KRTADGSEFESPKKKRKV

SEQ ID NO: 191 - protein, 16 aa, synthetic construct
  REGION 1..16: note = synthetic peptide
KRPAATKKAGQAKKKK

SEQ ID NO: 192 - protein, 17 aa, synthetic construct
  REGION 1..17: note = synthetic peptide
KKTELQTTNAENKTKKL

SEQ ID NO: 193 - protein, 19 aa, synthetic construct
  REGION 1..19: note = synthetic peptide
KRGINDRNFWRGENGRKTR

SEQ ID NO: 194 - protein, 16 aa, synthetic construct
  REGION 1..16: note = synthetic peptide
RKSGKIAAIVVKRPRK

SEQ ID NO: 195 - protein, 7 aa, synthetic construct
  REGION 1..7: note = synthetic peptide
PKKKRKV

SEQ ID NO: 196 - protein, 30 aa, synthetic construct
  REGION 1..30: note = synthetic polypeptide
MDSLLMNRRKFLYQFKNVRWAKGRRETYLC

SEQ ID NO: 197 - protein, 1368 aa, Streptococcus pyogenes [sequence omitted]

SEQ ID NO: 198 - DNA, 4104 bp, Streptococcus pyogenes
  misc_feature 1..4104: note = wild type spCas9 polynucleotide sequence
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 199 - protein, 1367 aa, Streptococcus pyogenes
  REGION 1..1367: note = MISC_FEATURE - spCas9 polypeptide sequence
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 200 - DNA, 4212 bp, synthetic construct
  misc_feature 1..4212: note = wild-type Cas9 polynucleotide sequence
  misc_feature 5..58: note = RuvC domain
  misc_feature 718..764: note = RuvC domain
  misc_feature 772..901: note = HNH domain
  misc_feature 906..1101: note = RuvC domain
  source 1..4212: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 201 - protein, 1368 aa, synthetic construct
  REGION 1..1368: note = exemplary catalytically active Cas9 nickase (nCas9) polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 202 - DNA, 4107 bp, Streptococcus pyogenes
  misc_feature 1..4107: note = Cas9 from Streptococcus pyogenes (NCBI Ref. Seq.: NC_002737.2)polynucleotide sequence
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 203 - protein, 1368 aa, synthetic construct
  REGION 1..1368: note = catalytically inactive Cas9 (dCas9)polypeptide sequence
  REGION 5..58: note = MISC_FEATURE - RuvC domain
  REGION 718..764: note = MISC_FEATURE - RuvC domain
  REGION 773..902: note = MISC_FEATURE - HNH domain
  REGION 907..1102: note = MISC_FEATURE - RuvC domain
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 204 - protein, 345 aa, Sulfolobus islandicus
  REGION 1..345: note = MISC_FEATURE - tr|F0NN87|F0NN87_SULIHCRISPR-associatedCasx protein OS =Sulfolobus islandicus (strain HVE10/4) GN = SiH_0402 PE=4; SV = 1);CasX polypeptide sequence
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYEFGRSPGMVERTRRVKLEVEPHYLIIAAAGWVLTRLGKAKVSEGDYVGVNVFTPTRGILYSLIQNVNGIVPGIKPETAFGLWIARKVVSSVTNPNVSVVRIYTISDAVGQNPTTINGGFSIDLTKLLEKRYLLSERLEAIARNALSISSNMRERYIVLANYIYEYLTGSKRLEDLLYFANRDLIMNLNSDDGKVRDLKLISAYVNGELIRGEG

SEQ ID NO: 205 - protein, 345 aa, Sulfolobus islandicus
  REGION 1..345: note = MISC_FEATURE - tr|F0NH53|F0NH53_SULIR CRISPR associated protein, Casx OS =Sulfolobus islandicus (strain REY15A) GN=SiRe_0771 PE=4 SV=1);CasX polypeptide sequence
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYKFGRSPGMVERTRRVKLEVEPHYLIMAAAGWVLTRLGKAKVSEGDYVGVNVFTPTRGILYSLIQNVNGIVPGIKPETAFGLWIARKVVSSVTNPNVSVVSIYTISDAVGQNPTTINGGFSIDLTKLLEKRDLLSERLEAIARNALSISSNMRERYIVLANYIYEYLTGSKRLEDLLYFANRDLIMNLNSDDGKVRDLKLISAYVNGELIRGEG

SEQ ID NO: 206 - protein, 987 aa, unidentified
  REGION 1..987: note = MISC_FEATURE - CasX polypeptide sequence
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPVKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDFAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVARGENIPAVIALTDPEGCPLPEFKDSSGGPTDILRIGEGYKEKQRAIQAAKEVEQRRAGGYSRKFASKSRNLADDMVRNSARDLFYHAVTHDAVLVFANLSRGFGRQGKRTFMTERQYTKMEDWLTAKLAYEGLTSKTYLSKTLAQYTSKTCSNCGFTITYADMDVMLVRLKKTSDGWATTLNNKELKAEYQITYYNRYKRQTVEKELSAELDRLSEESGNNDISKWTKGRRDEALFLLKKRFSHRPVQEQFVCLDCGHEVHAAEQAALNIARSWLFLNSNSTEFKSYKSGKQPFVGAWQAFYKRRLKEVWKPNA

SEQ ID NO: 207 - protein, 1210 aa, unidentified
  REGION 1..1210: note = MISC_FEATURE - APG80656.1 CRISPR-associated protein CasY [uncultured Parcubacteria group bacterium]; CasY polypeptide sequence
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 208 - protein, 1082 aa, Neisseria meningitidis
  REGION 1..1082: note = MISC_FEATURE - NCBI Reference: WP_002235162.1; type II CRISPR RNA-guided endonuclease Cas9; Nme1Cas9 polypeptide sequence
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 209 - protein, 1082 aa, Neisseria meningitidis
  REGION 1..1082: note = MISC_FEATURE - NCBI Reference: WP_002230835; type II CRISPR RNA-guided endonuclease Cas9; Nme2Cas9 polypeptide sequence
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 210 - protein, 1300 aa, Francisella tularensis
  REGION 1..1300: note = MISC_FEATURE - wild type Cpf1 polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 211 - protein, 1300 aa, Francisella tularensis
  REGION 1..1300: note = MISC_FEATURE - Cpf1 D917A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 212 - protein, 1300 aa, Francisella tularensis
  REGION 1..1300: note = MISC_FEATURE - Cpf1 E1006A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 213 - protein, 1300 aa, Francisella tularensis
  REGION 1..1300: note = MISC_FEATURE - Cpf1 D1255A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 214 - protein, 1300 aa, Francisella tularensis
  REGION 1..1300: note = MISC_FEATURE - Cpf1 D917A/E1006A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 215 - protein, 1300 aa, Francisella tularensis
  REGION 1..1300: note = MISC_FEATURE - Cpf1 D917A/D1255A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 216 - protein, 1300 aa, Francisella tularensis
  REGION 1..1300: note = MISC_FEATURE - Cpf1 E1006A/D1255A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 217 - protein, 1300 aa, Francisella tularensis
  REGION 1..1300: note = MISC_FEATURE - Cpf1 D917A/E1006A/D1255A polypeptide sequence
  source 1..1300: note = subspecies novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 218 - protein, 1052 aa, Staphylococcus aureus
  REGION 1..1052: note = MISC_FEATURE - Exemplary SaCas9 polypeptide sequence
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 219 - protein, 1052 aa, Staphylococcus aureus
  REGION 1..1052: note = MISC_FEATURE - SaCas9n polypeptide sequence
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 220 - protein, 1052 aa, Staphylococcus aureus
  REGION 1..1052: note = MISC_FEATURE - SaKKH Cas9 polypeptide sequence
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 221 - protein, 707 aa, synthetic construct
  REGION 1..707: note = Casphi-1 polypeptide sequence
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 222 - protein, 757 aa, synthetic construct
  REGION 1..757: note = Casphi-2 polypeptide sequence
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 223 - protein, 766 aa, synthetic construct
  REGION 1..766: note = Casphi-3 polypeptide sequence
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 224 - protein, 765 aa, synthetic construct
  REGION 1..765: note = Casphi-4 polypeptide sequence
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 225 - protein, 793 aa, synthetic construct
  REGION 1..793: note = Casphi-5 polypeptide sequence
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 226 - protein, 441 aa, synthetic construct
  REGION 1..441: note = Casphi-6 polypeptide sequence
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 227 - protein, 772 aa, synthetic construct
  REGION 1..772: note = Casphi-7 polypeptide sequence
MSNTAVSTREHMSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 228 - protein, 717 aa, synthetic construct
  REGION 1..717: note = Casphi-8 polypeptide sequence
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 229 - protein, 793 aa, synthetic construct
  REGION 1..793: note = Casphi-9 polypeptide sequence
MRSSREIGDKILMRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 230 - protein, 812 aa, synthetic construct
  REGION 1..812: note = Casphi-10 polypeptide sequence
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 231 - protein, 84 aa, synthetic construct
  REGION 1..84: note = synthetic polypeptide sequence
MTNLSDIIEKETGKQLVIQESILMLPEEVEEVIGNKPESDILVHTAYDESTDENVMLLTSDAPEYKPWALVIQDSNGENKIKML

SEQ ID NO: 232 - protein, 1129 aa, Alicyclobacillus acidoterrestris
  REGION 1..1129: note = MISC_FEATURE - Cas12b/C2c1; sp|T0D7A2|C2C1_ALIAG CRISPR-associated endonuclease C2c1 OS = Alicyclobacillus acido-terrestris (strain ATCC 49025 /DSM 3922/ CIP 106132 / NCIMB 13137/GD3B) GN=c2c1 PE=1 SV=1; Cas12b/C2c1 polypeptide sequence
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 233 - protein, 1368 aa, Mus musculus
  REGION 1..1368: note = MISC_FEATURE - high fidelity Cas9 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 234 - protein, 1368 aa, Streptococcus pyogenes
  REGION 1..1368: note = MISC_FEATURE - PAM-binding SpEQR Cas9 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESVLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 235 - protein, 1368 aa, Streptococcus pyogenes
  REGION 1..1368: note = MISC_FEATURE - PAM-binding SpVQR Cas9 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 236 - protein, 1368 aa, Streptococcus pyogenes
  REGION 1..1368: note = MISC_FEATURE - SpVQR Cas9 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 237 - protein, 1359 aa, Streptococcus macacae
  REGION 1..1359: note = MISC_FEATURE - SpyMacCas9 polypeptide sequence
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEIQTVGQNGGLFDDNPKSPLEVTPSKLVPLKKELNPKKYGGYQKPTTAYPVLLITDTKQLIPISVMNKKQFEQNPVKFLRDRGYQQVGKNDFIKLPKYTLVDIGDGIKRLWASSKEIHKGNQLVVSKKSQILLYHAHHLDSDLSNDYLQNHNQQFDVLFNEIISFSKKCKLGKEHIQKIENVYSNKKNSASIEELAESFIKLLGFTQLGATSPFNFLGVKLNQKQYKGKKDYILPCTEGTLIRQSITGLYETRVDLSKIGED

SEQ ID NO: 238 - protein, 1410 aa, synthetic construct
  REGION 1..1410: note = CP5 polypeptide sequence
  REGION 1..341: note = MISC_FEATURE - Sequence derived from Cas9
  REGION 342..362: note = MISC_FEATURE - Linker sequence
  REGION 363..1388: note = MISC_FEATURE - Sequence derived from Cas9
  REGION 1389..1410: note = MISC_FEATURE - Bipartite nuclear localization sequence
EIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEGADKRTADGSEFESPKKKRKV

SEQ ID NO: 239 - protein, 1302 aa, synthetic construct
  REGION 1..1302: note = Cas12c1 polypeptide sequence
MQTKKTHLHLISAKASRKYRRTIACLSDTAKKDLERRKQSGAADPAQELSCLKTIKFKLEVPEGSKLPSFDRISQIYNALETIEKGSLSYLLFALILSGFRIFPNSSAAKTFASSSCYKNDQFASQIKEIFGEMVKNFIPSELESILKKGRRKNNKDWTEENIKRVLNSEFGRKNSEGSSALFDSFLSKFSQELFRKFDSWNEVNKKYLEAAELLDSMLASYGPFDSVCKMIGDSDSRNSLPDKSTIAFTNNAEITVDIESSVMPYMAIAALLREYRQSKSKAAPVAYVQSHLTTTNGNGLSWFFKFGLDLIRKAPVSSKQSTSDGSKSLQELFSVPDDKLDGLKFIKEACEALPEASLLCGEKGELLGYQDFRTSFAGHIDSWVANYVNRLFELIELVNQLPESIKLPSILTQKNHNLVASLGLQEAEVSHSLELFEGLVKNVRQTLKKLAGIDISSSPNEQDIKEFYAFSDVLNRLGSIRNQIENAVQTAKKDKIDLESAIEWKEWKKLKKLPKLNGLGGGVPKQQELLDKALESVKQIRHYQRIDFERVIQWAVNEHCLETVPKFLVDAEKKKINKESSTDFAAKENAVRFLLEGIGAAARGKTDSVSKAAYNWFVVNNFLAKKDLNRYFINCQGCIYKPPYSKRRSLAFALRSDNKDTIEVVWEKFETFYKEISKEIEKFNIFSQEFQTFLHLENLRMKLLLRRIQKPIPAEIAFFSLPQEYYDSLPPNVAFLALNQEITPSEYITQFNLYSSFLNGNLILLRRSRSYLRAKFSWVGNSKLIYAAKEARLWKIPNAYWKSDEWKMILDSNVLVFDKAGNVLPAPTLKKVCEREGDLRLFYPLLRQLPHDWCYRNPFVKSVGREKNVIEVNKEGEPKVASALPGSLFRLIGPAPFKSLLDDCFFNPLDKDLRECMLIVDQEISQKVEAQKVEASLESCTYSIAVPIRYHLEEPKVSNQFENVLAIDQGEAGLAYAVFSLKSIGEAETKPIAVGTIRIPSIRRLIHSVSTYRKKKQRLQNFKQNYDSTAFIMRENVTGDVCAKIVGLMKEFNAFPVLEYDVKNLESGSRQLSAVYKAVNSHFLYFKEPGRDALRKQLWYGGDSWTIDGIEIVTRERKEDGKEGVEKIVPLKVFPGRSVSARFTSKTCSCCGRNVFDWLFTEKKAKTNKKFNVNSKGELTTADGVIQLFEADRSKGPKFYARRKERTPLTKPIAKGSYSLEEIERRVRTNLRRAPKSKQSRDTSQSQYFCVYKDCALHFSGMQADENAAINIGRRFLTALRKNRRSDFPSNVKISDRLLDN

SEQ ID NO: 240 - protein, 1218 aa, synthetic construct
  REGION 1..1218: note = Cas12c2 polypeptide sequence
MTKHSIPLHAFRNSGADARKWKGRIALLAKRGKETMRTLQFPLEMSEPEAAAINTTPFAVAYNAIEGTGKGTLFDYWAKLHLAGFRFFPSGGAATIFRQQAVFEDASWNAAFCQQSGKDWPWLVPSKLYERFTKAPREVAKKDGSKKSIEFTQENVANESHVSLVGASITDKTPEDQKEFFLKMAGALAEKFDSWKSANEDRIVAMKVIDEFLKSEGLHLPSLENIAVKCSVETKPDNATVAWHDAPMSGVQNLAIGVFATCASRIDNIYDLNGGKLSKLIQESATTPNVTALSWLFGKGLEYFRTTDIDTIMQDFNIPASAKESIKPLVESAQAIPTMTVLGKKNYAPFRPNFGGKIDSWIANYASRLMLLNDILEQIEPGFELPQALLDNETLMSGIDMTGDELKELIEAVYAWVDAAKQGLATLLGRGGNVDDAVQTFEQFSAMMDTLNGTLNTISARYVRAVEMAGKDEARLEKLIECKFDIPKWCKSVPKLVGISGGLPKVEEEIKVMNAAFKDVRARMFVRFEEIAAYVASKGAGMDVYDALEKRELEQIKKLKSAVPERAHIQAYRAVLHRIGRAVQNCSEKTKQLFSSKVIEMGVFKNPSHLNNFIFNQKGAIYRSPFDRSRHAPYQLHADKLLKNDWLELLAEISATLMASESTEQMEDALRLERTRLQLQLSGLPDWEYPASLAKPDIEVEIQTALKMQLAKDTVTSDVLQRAFNLYSSVLSGLTFKLLRRSFSLKMRFSVADTTQLIYVPKVCDWAIPKQYLQAEGEIGIAARVVTESSPAKMVTEVEMKEPKALGHFMQQAPHDWYFDASLGGTQVAGRIVEKGKEVGKERKLVGYRMRGNSAYKTVLDKSLVGNTELSQCSMIIEIPYTQTVDADFRAQVQAGLPKVSINLPVKETITASNKDEQMLFDRFVAIDLGERGLGYAVFDAKTLELQESGHRPIKAITNLLNRTHHYEQRPNQRQKFQAKFNVNLSELRENTVGDVCHQINRICAYYNAFPVLEYMVPDRLDKQLKSVYESVTNRYIWSSTDAHKSARVQFWLGGETWEHPYLKSAKDKKPLVLSPGRGASGKGTSQTCSCCGRNPFDLIKDMKPRAKIAVVDGKAKLENSELKLFERNLESKDDMLARRHRNERAGMEQPLTPGNYTVDEIKALLRANLRRAPKNRRTKDTTVSEYHCVFSDCGKTMHADENAAVNIGGKFIADIEK

SEQ ID NO: 241 - protein, 1252 aa, Oleiphilus sp.
  REGION 1..1252: note = MISC_FEATURE - OspCas12c polypeptide sequence
MTKLRHRQKKLTHDWAGSKKREVLGSNGKLQNPLLMPVKKGQVTEFRKAFSAYARATKGEMTDGRKNMFTHSFEPFKTKPSLHQCELADKAYQSLHSYLPGSLAHFLLSAHALGFRIFSKSGEATAFQASSKIEAYESKLASELACVDLSIQNLTISTLFNALTTSVRGKGEETSADPLIARFYTLLTGKPLSRDTQGPERDLAEVISRKIASSFGTWKEMTANPLQSLQFFEEELHALDANVSLSPAFDVLIKMNDLQGDLKNRTIVFDPDAPVFEYNAEDPADIIIKLTARYAKEAVIKNQNVGNYVKNAITTTNANGLGWLLNKGLSLLPVSTDDELLEFIGVERSHPSCHALIELIAQLEAPELFEKNVFSDTRSEVQGMIDSAVSNHIARLSSSRNSLSMDSEELERLIKSFQIHTPHCSLFIGAQSLSQQLESLPEALQSGVNSADILLGSTQYMLTNSLVEESIATYQRTLNRINYLSGVAGQINGAIKRKAIDGEKIHLPAAWSELISLPFIGQPVIDVESDLAHLKNQYQTLSNEFDTLISALQKNFDLNFNKALLNRTQHFEAMCRSTKKNALSKPEIVSYRDLLARLTSCLYRGSLVLRRAGIEVLKKHKIFESNSELREHVHERKHFVFVSPLDRKAKKLLRLTDSRPDLLHVIDEILQHDNLENKDRESLWLVRSGYLLAGLPDQLSSSFINLPIITQKGDRRLIDLIQYDQINRDAFVMLVTSAFKSNLSGLQYRANKQSFVVTRTLSPYLGSKLVYVPKDKDWLVPSQMFEGRFADILQSDYMVWKDAGRLCVIDTAKHLSNIKKSVFSSEEVLAFLRELPHRTFIQTEVRGLGVNVDGIAFNNGDIPSLKTFSNCVQVKVSRTNTSLVQTLNRWFEGGKVSPPSIQFERAYYKKDDQIHEDAAKRKIRFQMPATELVHASDDAGWTPSYLLGIDPGEYGMGLSLVSINNGEVLDSGFIHINSLINFASKKSNHQTKVVPRQQYKSPYANYLEQSKDSAAGDIAHILDRLIYKLNALPVFEALSGNSQSAADQVWTKVLSFYTWGDNDAQNSIRKQHWFGASHWDIKGMLRQPPTEKKPKPYIAFPGSQVSSYGNSQRCSCCGRNPIEQLREMAKDTSIKELKIRNSEIQLFDGTIKLFNPDPSTVIERRRHNLGPSRIPVADRTFKNISPSSLEFKELITIVSRSIRHSPEFIAKKRGIGSEYFCAYSDCNSSLNSEANAAANVAQKFQKQLFFEL

SEQ ID NO: 242 - protein, 767 aa, synthetic construct
  REGION 1..767: note = Cas12g1 polypeptide sequence
MAQASSTPAVSPRPRPRYREERTLVRKLLPRPGQSKQEFRENVKKLRKAFLQFNADVSGVCQWAIQFRPRYGKPAEPTETFWKFFLEPETSLPPNDSRSPEFRRLQAFEAAAGINGAAALDDPAFTNELRDSILAVASRPKTKEAQRLFSRLKDYQPAHRMILAKVAAEWIESRYRRAHQNWERNYEEWKKEKQEWEQNHPELTPEIREAFNQIFQQLEVKEKRVRICPAARLLQNKDNCQYAGKNKHSVLCNQFNEFKKNHLQGKAIKFFYKDAEKYLRCGLQSLKPNVQGPFREDWNKYLRYMNLKEETLRGKNGGRLPHCKNLGQECEFNPHTALCKQYQQQLSSRPDLVQHDELYRKWRREYWREPRKPVFRYPSVKRHSIAKIFGENYFQADFKNSVVGLRLDSMPAGQYLEFAFAPWPRNYRPQPGETEISSVHLHFVGTRPRIGFRFRVPHKRSRFDCTQEELDELRSRTFPRKAQDQKFLEAARKRLLETFPGNAEQELRLLAVDLGTDSARAAFFIGKTFQQAFPLKIVKIEKLYEQWPNQKQAGDRRDASSKQPRPGLSRDHVGRHLQKMRAQASEIAQKRQELTGTPAPETTTDQAAKKATLQPFDLRGLTVHTARMIRDWARLNARQIIQLAEENQVDLIVLESLRGFRPPGYENLDQEKKRRVAFFAHGRIRRKVTEKAVERGMRVVTVPYLASSKVCAECRKKQKDNKQWEKNKKRGLFKCEGCGSQAQVDENAARVLGRVFWGEIELPTAIP

SEQ ID NO: 243 - protein, 870 aa, synthetic construct
  REGION 1..870: note = Cas12h1 polypeptide sequence
MKVHEIPRSQLLKIKQYEGSFVEWYRDLQEDRKKFASLLFRWAAFGYAAREDDGATYISPSQALLERRLLLGDAEDVAIKFLDVLFKGGAPSSSCYSLFYEDFALRDKAKYSGAKREFIEGLATMPLDKIIERIRQDEQLSKIPAEEWLILGAEYSPEEIWEQVAPRIVNVDRSLGKQLRERLGIKCRRPHDAGYCKILMEVVARQLRSHNETYHEYLNQTHEMKTKVANNLTNEFDLVCEFAEVLEEKNYGLGWYVLWQGVKQALKEQKKPTKIQIAVDQLRQPKFAGLLTAKWRALKGAYDTWKLKKRLEKRKAFPYMPNWDNDYQIPVGLTGLGVFTLEVKRTEVVVDLKEHGKLFCSHSHYFGDLTAEKHPSRYHLKFRHKLKLRKRDSRVEPTIGPWIEAALREITIQKKPNGVFYLGLPYALSHGIDNFQIAKRFFSAAKPDKEVINGLPSEMVVGAADLNLSNIVAPVKARIGKGLEGPLHALDYGYGELIDGPKILTPDGPRCGELISLKRDIVEIKSAIKEFKACQREGLTMSEETTTWLSEVESPSDSPRCMIQSRIADTSRRLNSFKYQMNKEGYQDLAEALRLLDAMDSYNSLLESYQRMHLSPGEQSPKEAKFDTKRASFRDLLRRRVAHTIVEYFDDCDIVFFEDLDGPSDSDSRNNALVKLLSPRTLLLYIRQALEKRGIGMVEVAKDGTSQNNPISGHVGWRNKQNKSEIYFYEDKELLVMDADEVGAMNILCRGLNHSVCPYSFVTKAPEKKNDEKKEGDYGKRVKRFLKDRYGSSNVRFLVASMGFVTVTTKRPKDALVGKRLYYHGGELVTHDLHNRMKDEIKYLVEKEVLARRVSLSDSTIKSYKSFAHV

SEQ ID NO: 244 - protein, 1093 aa, synthetic construct
  REGION 1..1093: note = Cas12i1 polypeptide sequence
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 245 - protein, 1054 aa, synthetic construct
  REGION 1..1054: note = Cas12i2 polypeptide sequence
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 246 - protein, 4 aa, synthetic construct
  REGION 1..4: note = synthetic peptide sequence
GGGS

SEQ ID NO: 247 - protein, 5 aa, synthetic construct
  REGION 1..5: note = synthetic peptide sequence
GGGGS

SEQ ID NO: 248 - protein, 5 aa, synthetic construct
  REGION 1..5: note = synthetic peptide sequence
EAAAK

SEQ ID NO: 249 - protein, 16 aa, synthetic construct
  REGION 1..16: note = synthetic peptide sequence
SGSETPGTSESATPES

SEQ ID NO: 250 - protein, 6 aa, synthetic construct
  REGION 1..6: note = linker peptide sequence
GGSGGS

SEQ ID NO: 251 - protein, 20 aa, synthetic construct
  REGION 1..20: note = synthetic peptide sequence
GSSGSETPGTSESATPESSG

SEQ ID NO: 252 - DNA, 18 bp, synthetic construct
  misc_feature 1..18: note = synthetic oligonucleotide sequence
  source 1..18: mol_type = other DNA
ggaggctctggaggaagc

SEQ ID NO: 253 - DNA, 60 bp, synthetic construct
  misc_feature 1..60: note = synthetic oligonucleotide sequence
  source 1..60: mol_type = other DNA
ggctcttctggatctgaaacacctggcacaagcgagagcgccacccctgagagctctggc

SEQ ID NO: 254 - protein, 1129 aa, Alicyclobacillus acidiphilus
  REGION 1..1129: note = MISC_FEATURE - AacCas12b polypeptide sequence
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 255 - protein, 1140 aa, Bacillus hisashii
  REGION 1..1140: note = MISC_FEATURE - BhCas12b polypeptide sequence
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKK

SEQ ID NO: 256 - protein, 1108 aa, Bacillus thermoamylovorans
  REGION 1..1108: note = MISC_FEATURE - BTCas12b polypeptide sequence
MATRSFILKIEPNEEVKKGLWKTHEVLNHGIAYYMNILKLIRQEAIYEHHEQDPKNPKKVSKAEIQAELWDFVLKMQKCNSFTHEVDKDVVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPFTDSNEPIVKEIKWMEKSRNQSVRRLDKDMFIQALERFLSWESWNLKVKEEYEKVEKEHKTLEERIKEDIQAFKSLEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKFVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKLVTTHADINAAQNLQKRFWTRTHGFYKVYCKAYQVDGQTVYIPESKDQKQKIIEEFGEGYFILKDGVYEWGNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 257 - protein, 1112 aa, Bacillus sp.
  REGION 1..1112: note = MISC_FEATURE - BvCas12b (Bacillus sp. V3-13) polypeptide sequence
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 258 - DNA, 47 bp, synthetic construct
  misc_feature 1..47: note = 5'UTR oligonucleotide sequence
  source 1..47: mol_type = other DNA
gggaaataagagagaaaagaagagtaagaagaaatataagagccacc

SEQ ID NO: 259 - DNA, 101 bp, synthetic construct
  misc_feature 1..101: note = 3'UTR (TriLink standard UTR)polynucleotide sequence
  source 1..101: mol_type = other DNA
gctggagcctcggtggccatgcttcttgccccttgggcctccccccagcccctcctccccttcctgcacccgtacccccgtggtctttgaataaagtctga

SEQ ID NO: 260 - DNA, 3420 bp, Bacillus hisashii
  misc_feature 1..3420: note = bhCas12b (V4) polynucleotide sequence
  source 1..3420: mol_type = genomic DNA
atggccccaaagaagaagcggaaggtcggtatccacggagtcccagcagccgccaccagatccttcatcctgaagatcgagcccaacgaggaagtgaagaaaggcctctggaaaacccacgaggtgctgaaccacggaatcgcctactacatgaatatcctgaagctgatccggcaagaggccatctacgagcaccacgagcaggaccccaagaatcccaagaaggtgtccaaggccgagatccaggccgagctgtgggatttcgtgctgaagatgcagaagtgcaacagcttcacacacgaggtggacaaggacgaggtgttcaacatcctgagagagctgtacgaggaactggtgcccagcagcgtggaaaagaagggcgaagccaaccagctgagcaacaagtttctgtaccctctggtggaccccaacagccagtctggaaagggaacagccagcagcggcagaaagcccagatggtacaacctgaagattgccggcgatccctcctgggaagaagagaagaagaagtgggaagaagataagaaaaaggacccgctggccaagatcctgggcaagctggctgagtacggactgatccctctgttcatcccctacaccgacagcaacgagcccatcgtgaaagaaatcaagtggatggaaaagtcccggaaccagagcgtgcggcggctggataaggacatgttcattcaggccctggaacggttcctgagctgggagagctggaacctgaaagtgaaagaggaatacgagaaggtcgagaaagagtacaagaccctggaagagaggatcaaagaggacatccaggctctgaaggctctggaacagtatgagaaagagcggcaagaacagctgctgcgggacaccctgaacaccaacgagtaccggctgagcaagagaggccttagaggctggcgggaaatcatccagaaatggctgaaaatggacgagaacgagccctccgagaagtacctggaagtgttcaaggactaccagcggaagcaccctagagaggccggcgattacagcgtgtacgagttcctgtccaagaaagagaaccacttcatctggcggaatcaccctgagtacccctacctgtacgccaccttctgcgagatcgacaagaaaaagaaggacgccaagcagcaggccaccttcacactggccgatcctatcaatcaccctctgtgggtccgattcgaggaaagaagcggcagcaacctgaacaagtacagaatcctgaccgagcagctgcacaccgagaagctgaagaaaaagctgacagtgcagctggaccggctgatctaccctacagaatctggcggctgggaagagaagggcaaagtggacattgtgctgctgcccagccggcagttctacaaccagatcttcctggacatcgaggaaaagggcaagcacgccttcacctacaaggatgagagcatcaagttccctctgaagggcacactcggcggagccagagtgcagttcgacagagatcacctgagaagataccctcacaaggtggaaagcggcaacgtgggcagaatctacttcaacatgaccgtgaacatcgagcctacagagtccccagtgtccaagtctctgaagatccaccgggacgacttccccaaggtggtcaacttcaagcccaaagaactgaccgagtggatcaaggacagcaagggcaagaaactgaagtccggcatcgagtccctggaaatcggcctgagagtgatgagcatcgacctgggacagagacaggccgctgccgcctctattttcgaggtggtggatcagaagcccgacatcgaaggcaagctgtttttcccaatcaagggcaccgagctgtatgccgtgcacagagccagcttcaacatcaagctgcccggcgagacactggtcaagagcagagaagtgctgcggaaggccagagaggacaatctgaaactgatgaaccagaagctcaacttcctgcggaacgtgctgcacttccagcagttcgaggacatcaccgagagagagaagcgggtcaccaagtggatcagcagacaagagaacagcgacgtgcccctggtgtaccaggatgagctgatccagatccgcgagctgatgtacaagccttacaaggactgggtcgccttcctgaagcagctccacaagagactggaagtcgagatcggcaaagaagtgaagcactggcggaagtccctgagcgacggaagaaagggcctgtacggcatctccctgaagaacatcgacgagatcgatcggacccggaagttcctgctgagatggtccctgaggcctaccgaacctggcgaagtgcgtagactggaacccggccagagattcgccatcgaccagctgaatcacctgaacgccctgaaagaagatcggctgaagaagatggccaacaccatcatcatgcacgccctgggctactgctacgacgtgcggaagaagaaatggcaggctaagaaccccgcctgccagatcatcctgttcgaggatctgagcaactacaacccctacgaggaaaggtcccgcttcgagaacagcaagctcatgaagtggtccagacgcgagatccccagacaggttgcactgcagggcgagatctatggcctgcaagtgggagaagtgggcgctcagttcagcagcagattccacgccaagacaggcagccctggcatcagatgtagcgtcgtgaccaaagagaagctgcaggacaatcggttcttcaagaatctgcagagagagggcagactgaccctggacaaaatcgccgtgctgaaagagggcgatctgtacccagacaaaggcggcgagaagttcatcagcctgagcaaggatcggaagtgcgtgaccacacacgccgacatcaacgccgctcagaacctgcagaagcggttctggacaagaacccacggcttctacaaggtgtactgcaaggcctaccaggtggacggccagaccgtgtacatccctgagagcaaggaccagaagcagaagatcatcgaagagttcggcgagggctacttcattctgaaggacggggtgtacgaatgggtcaacgccggcaagctgaaaatcaagaagggcagctccaagcagagcagcagcgagctggtggatagcgacatcctgaaagacagcttcgacctggcctccgagctgaaaggcgaaaagctgatgctgtacagggaccccagcggcaatgtgttccccagcgacaaatggatggccgctggcgtgttcttcggaaagctggaacgcatcctgatcagcaagctgaccaaccagtactccatcagcaccatcgaggacgacagcagcaagcagtctatgaaaaggccggcggccacgaaaaaggccggccaggcaaaaaagaaaaag

SEQ ID NO: 261 - protein, 17 aa, synthetic construct
  REGION 1..17: note = synthetic peptide sequence
MAPKKKRKVGIHGVPAA

SEQ ID NO: 262 - DNA, 51 bp, synthetic construct
  misc_feature 1..51: note = synthetic oligonucleotide sequence
  source 1..51: mol_type = other DNA
atggccccaaagaagaagcggaaggtcggtatccacggagtcccagcagcc

SEQ ID NO: 263 - protein, 1554 aa, synthetic construct
  REGION 1..1554: note = 101 Cas9 TadAins 1015 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 264 - protein, 1554 aa, synthetic construct
  REGION 1..1554: note = 102 Cas9 TadAins 1022 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 265 - protein, 1554 aa, synthetic construct
  REGION 1..1554: note = 103 Cas9 TadAins 1029 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 266 - protein, 1554 aa, synthetic construct
  REGION 1..1554: note = 103 Cas9 TadAins 1040 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 267 - protein, 1554 aa, synthetic construct
  REGION 1..1554: note = 105 Cas9 TadAins 1068 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGEGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDTGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 268 - protein, 1554 aa, synthetic construct
  REGION 1..1554: note = 106 Cas9 TadAins 1247 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 269 - protein, 1554 aa, synthetic construct
  REGION 1..1554: note = 107 Cas9 TadAins 1054 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 270 - protein, 1554 aa, synthetic construct
  REGION 1..1554: note = 108 Cas9 TadAins 1026 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 271 - protein, 1540 aa, synthetic construct
  REGION 1..1540: note = 109 Cas9 TadAins 768 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 272 - protein, 1540 aa, synthetic construct
  REGION 1..1540: note = 110.1 Cas9 TadAins 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPREDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 273 - protein, 1543 aa, synthetic construct
  REGION 1..1543: note = 110.2 Cas9 TadAins 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPREDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 274 - protein, 1546 aa, synthetic construct
  REGION 1..1546: note = 110.3 Cas9 TadAins 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSSGSETPGTSESATPESGSSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPREDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 275 - protein, 1546 aa, synthetic construct
  REGION 1..1546: note = 110.4 Cas9 TadAins 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSSGSETPGTSESATPESGSSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRREDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 276 - protein, 1546 aa, synthetic construct
  REGION 1..1546: note = 110.5 Cas9 TadAins 1249 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSGSSGSSGSETPGTSESATPESGSSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 277 - protein, 1551 aa, synthetic construct
  REGION 1..1551: note = 110.5 Cas9 TadAins delta59-66 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSSGSETPGTSESATPESGSSGSEVEFSHEYWMRHALTLAKRARDEREVPVGAVLVLNNRVIGEGWNRAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 278 - protein, 1546 aa, synthetic construct
  REGION 1..1546: note = 110.6 Cas9 TadAins 1251 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEGSSGSSGSETPGTSESATPESGSSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 279 - protein, 1546 aa, synthetic construct
  REGION 1..1546: note = 110.7 Cas9 TadAins 1252 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDGSSGSSGSETPGTSESATPESGSSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 280 - protein, 1533 aa, synthetic construct
  REGION 1..1533: note = 110.8 Cas9 TadAins delta59-66 C-truncate 1250 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPGSSGSETPGTSESATPESSGSEVEFSHEYWMRHALTLAKRARDEREVPVGAVLVLNNRVIGEGWNRAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 281 - protein, 1549 aa, synthetic construct
  REGION 1..1549: note = 111.1 Cas9 TadAins 997 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 282 - protein, 1555 aa, synthetic construct
  REGION 1..1555: note = 111.2 Cas9 TadAins 997 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSSGSETPGTSESATPESSGGSSIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 283 - protein, 1421 aa, synthetic construct
  REGION 1..1421: note = 112 delta HNH TadA polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 284 - protein, 1552 aa, synthetic construct
  REGION 1..1552: note = 113 N-term single TadA helixtrunc 165-end polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 285 - protein, 1545 aa, synthetic construct
  REGION 1..1545: note = 114 N-term single TadA helixtrunc 165-end delta 59-65 polypeptide sequence
MSEVEFSHEYWMRHALTLAKRARDEREVPVGAVLVLNNRVIGEGWNRTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 286 - protein, 1541 aa, synthetic construct
  REGION 1..1541: note = 115.1 Cas9 TadAins 1004 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 287 - protein, 1541 aa, synthetic construct
  REGION 1..1541: note = 115.2 Cas9 TadAins 1005 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 288 - protein, 1541 aa, synthetic construct
  REGION 1..1541: note = 115.3 Cas9 TadAins 1006 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLEGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQSEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 289 - protein, 1541 aa, synthetic construct
  REGION 1..1541: note = 115.4 Cas9 TadAins 1007 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 290 - protein, 1541 aa, synthetic construct
  REGION 1..1541: note = 116.1 Cas9 TadAins C-term truncate 792 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 291 - protein, 1541 aa, synthetic construct
  REGION 1..1541: note = 116.2 Cas9 TadAins C-term truncate 791 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 292 - protein, 1541 aa, synthetic construct
  REGION 1..1541: note = 116.3 Cas9 TadAins C-term truncate 790 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKEGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQLGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 293 - protein, 1484 aa, synthetic construct
  REGION 1..1484: note = 117 Cas9 delta 1017-1069 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYSSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 294 - protein, 1554 aa, synthetic construct
  REGION 1..1554: note = 118 Cas9 TadA-CP116ins 1067 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 295 - protein, 1554 aa, synthetic construct
  REGION 1..1554: note = 119 Cas9 TadAins 701 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 296 - protein, 1554 aa, synthetic construct
  REGION 1..1554: note = 120 Cas9 TadACP136ins 1248 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 297 - protein, 1554 aa, synthetic construct
  REGION 1..1554: note = 121 Cas9 TadACP136ins 1052 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLAMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGNGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 298 - protein, 1554 aa, synthetic construct
  REGION 1..1554: note = 122 Cas9 TadACP136ins 1041 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 299 - protein, 1554 aa, synthetic construct
  REGION 1..1554: note = 123 Cas9 TadACP139ins 1299 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGDKPIREQAENIIHLFTLTNLGAPAAFKYFDTTIDRKRYTSTKEVLDATLIHQSITGLYETRIDLSQLGGD

SEQ ID NO: 300 - protein, 1454 aa, synthetic construct
  REGION 1..1454: note = 124 Cas9 delta 792-872TadAins polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 301 - protein, 1420 aa, synthetic construct
  REGION 1..1420: note = 125 Cas9 delta 792-906TadAins polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 302 - protein, 1554 aa, synthetic construct
  REGION 1..1554: note = 126 TadA CP65ins 1003 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 303 - protein, 1554 aa, synthetic construct
  REGION 1..1554: note = 127 TadA CP65ins 1016 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 304 - protein, 1554 aa, synthetic construct
  REGION 1..1554: note = 128 TadA CP65ins 1022 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMITAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 305 - protein, 1554 aa, synthetic construct
  REGION 1..1554: note = 129 TadA CP65ins 1029 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEITAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 306 - protein, 1554 aa, synthetic construct
  REGION 1..1554: note = 130 TadA CP65ins 1041 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 307 - protein, 1554 aa, synthetic construct
  REGION 1..1554: note = 131 TadA CP65ins 1054 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 308 - protein, 1554 aa, synthetic construct
  REGION 1..1554: note = 132 TadA CP65ins 1246 polypeptide sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDGSSGSETPGTSESATPESSGSEVEFSHEYWMRHALTLAKRARDEREVPVGAVLVLNNRVIGEGWNRAIGLHDPSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 309 - protein, 160 aa, Staphylococcus aureus
  REGION 1..160: note = MISC_FEATURE - TadA polypeptide sequence
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 310 - protein, 161 aa, Bacillus subtilis
  REGION 1..161: note = MISC_FEATURE - TadA polypeptide sequence
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 311 - protein, 183 aa, Salmonella enterica
  REGION 1..183: note = MISC_FEATURE - TadA polypeptide sequence
  source 1..183: note = serovar Typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 312 - protein, 164 aa, Shewanella putrefaciens
  REGION 1..164: note = MISC_FEATURE - TadA polypeptide sequence
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 313 - protein, 173 aa, Haemophilus influenzae
  REGION 1..173: note = MISC_FEATURE - TadA polypeptide sequence
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 314 - protein, 161 aa, Caulobacter crescentus
  REGION 1..161: note = MISC_FEATURE - TadA polypeptide sequence
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 315 - protein, 179 aa, Geobacter sulfurreducens
  REGION 1..179: note = MISC_FEATURE - TadA polypeptide sequence
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 316 - protein, 167 aa, synthetic construct
  REGION 1..167: note = TadA*8 polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTD

SEQ ID NO: 317 - RNA, 80 nt, synthetic construct
  misc_feature 1..80: note = gRNA scaffold polynucleotide sequence
  source 1..80: mol_type = other RNA
gttttagagctagaaatagcaagttaaaataaggctagtccgttatcaacttgaaaaagtggcaccgagtcggtgctttt

SEQ ID NO: 318 - RNA, 125 nt, synthetic construct
  misc_feature 1..125: note = gRNA scaffold polynucleotide sequence
  source 1..125: mol_type = other RNA
gtttttgtactctcaagatttaagtaactgtacaacgaaacttacacagttacttaaatcttgcagaagctacaaagataaggcttcatgccgaaatcaacaccctgtcattttatggcagggtg

SEQ ID NO: 319 - RNA, 76 nt, Streptococcus pyogenes
  misc_feature 1..76: note = S. pyogenes gRNA scaffold oligonucleotide sequence
  source 1..76: mol_type = other RNA
gttttagagctagaaatagcaagttaaaataaggctagtccgttatcaacttgaaaaagtggcaccgagtcggtgc

SEQ ID NO: 320 - RNA, 84 nt, Staphylococcus aureus
  misc_feature 1..84: note = S. aureus gRNA scaffold oligonucleotide sequence
  source 1..84: mol_type = other RNA
gttttagtactctgtaatgaaaattacagaatctactaaaacaaggcaaaatgccgtgtttatctcgtcaacttgttggcgaga

SEQ ID NO: 321 - RNA, 97 nt, Bacillus hisashii
  misc_feature 1..97: note = BhCas12b gRNA scaffold oligonucleotide sequence
  source 1..97: mol_type = other RNA
gttctgtcttttggtcaggacaaccgtctagctataagtgctgcagggtgtgagaaactcctattgctggacgatgtctcttacgaggcattagcac

SEQ ID NO: 322 - RNA, 88 nt, Bacillus sp.
  misc_feature 1..88: note = BvCas12b gRNA scaffold oligonucleotide sequence
  source 1..88: mol_type = other RNA
gacctatagggtcaatgaatctgtgcgtgtgccataagtaattaaaaattacccaccacaggagcacctgaaaacaggtgcttggcac

SEQ ID NO: 323 - RNA, 80 nt, synthetic construct
  misc_feature 1..80: note = gRNA scaffold nucleotide sequence; oligonucleotide sequence
  modified_base 78..80: mod_base = um
  misc_feature 78..80: note = In embodiments, a nucleotide(s) is linked to the preceding 5' nucleotide by a 3' phosphorothioate internucleotide linkage
  source 1..80: mol_type = other RNA
gttttagagctagaaatagcaagttaaaataaggctagtccgttatcaacttgaaaaagtgggaccgagtcggtgctttt

SEQ ID NO: 324 - RNA, 80 nt, synthetic construct; identical to SEQ ID NO: 317
  misc_feature 1..80: note = gRNA scaffold nucleotide sequence
  misc_feature 78..80: note = In embodiments, a nucleotide(s) is linked to the preceding 5' nucleotide by a 3' phosphorothioate internucleotide linkage
  modified_base 78..80: mod_base = um
  source 1..80: mol_type = other RNA

SEQ ID NO: 325 - RNA, 117 nt, Bacillus hisashii
  misc_feature 1..117: note = BhCas12b gRNA scaffold + guide sequence; oligonucleotide sequence
  misc_feature 1..97: note = BhCas12b sgRNA scaffold
  source 1..117: mol_type = other RNA
gttctgtcttttggtcaggacaaccgtctagctataagtgctgcagggtgtgagaaactcctattgctggacgatgtctcttacgaggcattagcacnnnnnnnnnnnnnnnnnnnn

SEQ ID NO: 326 - RNA, 108 nt, Bacillus sp.
  misc_feature 1..108: note = BvCas12b gRNA scaffold + guide sequence; oligonucleotide sequence
  misc_feature 1..88: note = BvCas12b gRNA scaffold
  source 1..108: mol_type = other RNA
gacctatagggtcaatgaatctgtgcgtgtgccataagtaattaaaaattacccaccacaggagcacctgaaaacaggtgcttggcacnnnnnnnnnnnnnnnnnnnn

SEQ ID NO: 327 - RNA, 119 nt, Alicyclobacillus acidiphilus
  misc_feature 1..119: note = AaCas12b gRNA scaffold + guide sequence; oligonucleotide sequence
  misc_feature 1..99: note = AaCas12b gRNA scaffold
  source 1..119: mol_type = other RNA
gtctaaaggacagaatttttcaacgggtgtgccaatggccactttccaggtggcaaagcccgttgaacttctcaaaaagaacgatctgagaagtggcacnnnnnnnnnnnnnnnnnnnn

SEQ ID NO: 328 - protein, 29 aa, synthetic construct
  REGION 1..29: note = synthetic polypeptide sequence
PKKKRKVEGADKRTADGSEFESPKKKRKV

SEQ ID NO: 329 - protein, 23 aa, synthetic construct
  REGION 1..23: note = synthetic peptide sequence
RKSGKIAAIVVKRPRKPKKKRKV

SEQ ID NO: 330 - protein, 8 aa, synthetic construct
  REGION 1..8: note = synthetic peptide sequence
SGGSSGGS

SEQ ID NO: 331 - protein, 1609 aa, synthetic construct
  REGION 1..1609: note = pNMG-B335ABE8.1_Y147T_CP5_NGC PAM_monomer polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTDSGGSSGGSSGSETPGTSESATPESSGGSSGGSEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEGADKRTADGSEFESPKKKRKV

SEQ ID NO: 332 - protein, 1807 aa, synthetic construct
  REGION 1..1807: note = pNMG-357_ABE8.14 with NGC PAMCP5 polypeptide sequence
  REGION 1..174: note = MISC_FEATURE - Adenosine deaminase sequence
  REGION 175..198: note = MISC_FEATURE - Linker sequence
  REGION 199..365: note = MISC_FEATURE - Adenosine deaminase sequence
  REGION 366..397: note = MISC_FEATURE - Linker sequence
  REGION 398..738: note = MISC_FEATURE - Sequence derived from Cas9
  REGION 739..759: note = MISC_FEATURE - Linker sequence
  REGION 760..1785: note = MISC_FEATURE - Sequence derived from Cas9
  REGION 1786..1807: note = MISC_FEATURE - Bipartite nuclear localization sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDGGSSGGSSGSETPGTSESATPESSGGSSGGSMSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTDSGGSSGGSSGSETPGTSESATPESSGGSSGGSEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEGADKRTADGSEFESPKKKRKV

SEQ ID NO: 333 - protein, 1588 aa, synthetic construct
  REGION 1..1588: note = ABE8.8-m polypeptide sequence
  REGION 1..167: note = MISC_FEATURE - Adenosine deaminase sequence
  REGION 168..199: note = MISC_FEATURE - Linker sequence
  REGION 200..1566: note = MISC_FEATURE - Sequence derived from Cas9
  REGION 1567..1588: note = MISC_FEATURE - Bipartite nuclear localization sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 334 - protein, 1786 aa, synthetic construct
  REGION 1..1786: note = ABE8.8-d polypeptide sequence
  REGION 1..167: note = MISC_FEATURE - Adenosine deaminase sequence
  REGION 168..199: note = MISC_FEATURE - Linker sequence
  REGION 200..365: note = MISC_FEATURE - Adenosine deaminase sequence
  REGION 366..397: note = MISC_FEATURE - Linker sequence
  REGION 398..1764: note = MISC_FEATURE - Sequence derived from Cas9
  REGION 1765..1786: note = MISC_FEATURE - Bipartite nuclear localization sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 335 - protein, 1588 aa, synthetic construct
  REGION 1..1588: note = ABE8.13-m polypeptide sequence
  REGION 1..167: note = MISC_FEATURE - Adenosine deaminase sequence
  REGION 168..199: note = MISC_FEATURE - Linker sequence
  REGION 200..1566: note = MISC_FEATURE - Sequence derived from Cas9
  REGION 1567..1588: note = MISC_FEATURE - Bipartite nuclear localization sequence
MSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 336 - protein, 1786 aa, synthetic construct
  REGION 1..1786: note = ABE8.13-d polypeptide sequence
  REGION 1..167: note = MISC_FEATURE - Adenosine deaminase sequence
  REGION 168..199: note = MISC_FEATURE - Linker sequence
  REGION 200..365: note = MISC_FEATURE - Adenosine deaminase sequence
  REGION 366..397: note = MISC_FEATURE - Linker sequence
  REGION 398..1764: note = MISC_FEATURE - Sequence derived from Cas9
  REGION 1765..1786: note = MISC_FEATURE - Bipartite nuclear localization sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 337 - protein, 1588 aa, synthetic construct
  REGION 1..1588: note = ABE8.17-m polypeptide sequence
  REGION 1..167: note = MISC_FEATURE - Adenosine deaminase sequence
  REGION 168..199: note = MISC_FEATURE - Linker sequence
  REGION 200..1566: note = MISC_FEATURE - Sequence derived from Cas9
  REGION 1567..1588: note = MISC_FEATURE - Bipartite nuclear localization sequence
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 338 - protein, 1786 aa, synthetic construct
  REGION 1..1786: note = ABE8.17-d polypeptide sequence
  REGION 1..167: note = MISC_FEATURE - Adenosine deaminase sequence
  REGION 168..199: note = MISC_FEATURE - Linker sequence
  REGION 200..365: note = MISC_FEATURE - Adenosine deaminase sequence
  REGION 366..397: note = MISC_FEATURE - Linker sequence
  REGION 398..1764: note = MISC_FEATURE - Sequence derived from Cas9
  REGION 1765..1786: note = MISC_FEATURE - Bipartite nuclear localization sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 339 - protein, 1588 aa, synthetic construct
  REGION 1..1588: note = ABE8.20-m polypeptide sequence
  REGION 1..167: note = MISC_FEATURE - Adenosine deaminase sequence
  REGION 168..199: note = MISC_FEATURE - Linker sequence
  REGION 200..1566: note = MISC_FEATURE - Sequence derived from Cas9
  REGION 1567..1588: note = MISC_FEATURE - Bipartite nuclear localization sequence
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 340 - protein, 1786 aa, synthetic construct
  REGION 1..1786: note = ABE8.20-d polypeptide sequence
  REGION 1..167: note = MISC_FEATURE - Adenosine deaminase sequence
  REGION 168..199: note = MISC_FEATURE - Linker sequence
  REGION 200..365: note = MISC_FEATURE - Adenosine deaminase sequence
  REGION 366..397: note = MISC_FEATURE - Linker sequence
  REGION 398..1764: note = MISC_FEATURE - Sequence derived from Cas9
  REGION 1765..1786: note = MISC_FEATURE - Bipartite nuclear localization sequence
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 341 - protein, 1588 aa, synthetic construct
  REGION 1..1588: note = 01. monoABE8.1_bpNLS + Y147T polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 342 - protein, 1588 aa, synthetic construct
  REGION 1..1588: note = 02. monoABE8.1_bpNLS + Y147R polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCRFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 343 - protein, 1588 aa, synthetic construct
  REGION 1..1588: note = 03. monoABE8.1_bpNLS + Q154S polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRSVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 344 - protein, 1588 aa, synthetic construct
  REGION 1..1588: note = 04. monoABE8.1_bpNLS + Y123H polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 345 - protein, 1588 aa, synthetic construct
  REGION 1..1588: note = 05. monoABE8.1_bpNLS + V82S polypeptide sequence
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 346 - protein, 1588 aa, synthetic construct
  REGION 1..1588: note = 06. monoABE8.1_bpNLS + T166R polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSRDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 347 - protein, 1588 aa, synthetic construct
  REGION 1..1588: note = 07. monoABE8.1_bpNLS + Q154R polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 348 - protein, 1588 aa, synthetic construct
  REGION 1..1588: note = 08. monoABE8.1_bpNLS +Y147R_Q154R_Y123H polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 349 - protein, 1588 aa, synthetic construct
  REGION 1..1588: note = 09. monoABE8.1_bpNLS +Y147R_Q154R_I76Y polypeptide sequence
MSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHYPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 350 - protein, 1588 aa, synthetic construct
  REGION 1..1588: note = 10. monoABE8.1_bpNLS +Y147R_Q154R_T166R polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCRFFRMPRRVFNAQKKAQSSRDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 351 - protein, 1588 aa, synthetic construct
  REGION 1..1588: note = 11. monoABE8.1_bpNLS +Y147T_Q154R polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 352 - protein, 1588 aa, synthetic construct
  REGION 1..1588: note = 12. monoABE8.1_bpNLS +Y147T_Q154S polypeptide sequence
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRSVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 353 - protein, 1588 aa, synthetic construct
  REGION 1..1588: note = 13. monoABE8.1_bpNLS +H123Y123H_Y147R_Q154R_I76Y polypeptide sequence
MSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 354 - protein, 1588 aa, synthetic construct
  REGION 1..1588: note = 14. monoABE8.1_bpNLS + V82S +Q154R polypeptide sequence
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 355 - protein, 4 aa, synthetic construct
  REGION 1..4: note = synthetic peptide sequence
SGGS

SEQ ID NO: 356 - protein, 24 aa, synthetic construct
  REGION 1..24: note = synthetic peptide sequence
SGGSSGSETPGTSESATPESSGGS

SEQ ID NO: 357 - protein, 32 aa, synthetic construct
  REGION 1..32: note = synthetic polypeptide sequence
SGGSSGGSSGSETPGTSESATPESSGGSSGGS

SEQ ID NO: 358 - protein, 104 aa, synthetic construct
  REGION 1..104: note = synthetic polypeptide sequence
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 359 - protein, 24 aa, synthetic construct
  REGION 1..24: note = synthetic peptide sequence
SGGSSGGSSGSETPGTSESATPES

SEQ ID NO: 360 - protein, 40 aa, synthetic construct
  REGION 1..40: note = synthetic polypeptide sequence
SGGSSGGSSGSETPGTSESATPESSGGSSGGSSGGSSGGS

SEQ ID NO: 361 - protein, 64 aa, synthetic construct
  REGION 1..64: note = synthetic polypeptide sequence
SGGSSGGSSGSETPGTSESATPESSGGSSGGSSGGSSGGSSGSETPGTSESATPESSGGSSGGS

SEQ ID NO: 362 - protein, 92 aa, synthetic construct
  REGION 1..92: note = synthetic polypeptide sequence
PGSPAGSPTSTEEGTSESATPESGPGTSTEPSEGSAPGSPAGSPTSTEEGTSTEPSEGSAPGTSTEPSEGSAPGTSESATPESGPGSEPATS

SEQ ID NO: 363 - protein, 5 aa, synthetic construct
  REGION 1..5: note = synthetic peptide sequence
PAPAP

SEQ ID NO: 364 - protein, 6 aa, synthetic construct
  REGION 1..6: note = synthetic peptide sequence
PAPAPA

SEQ ID NO: 365 - protein, 7 aa, synthetic construct
  REGION 1..7: note = synthetic peptide sequence
PAPAPAP

SEQ ID NO: 366 - protein, 8 aa, synthetic construct
  REGION 1..8: note = synthetic peptide sequence
PAPAPAPA

SEQ ID NO: 367 - protein, 9 aa, synthetic construct
  REGION 1..9: note = synthetic peptide sequence
PAPAPAPAP

SEQ ID NO: 368 - protein, 15 aa, synthetic construct
  REGION 1..15: note = synthetic peptide sequence
PAPAPAPAPAPAPAP

SEQ ID NO: 369 - protein, 21 aa, synthetic construct
  REGION 1..21: note = synthetic peptide sequence
PAPAPAPAPAPAPAPAPAPAP

SEQ ID NO: 370 - DNA, 306 bp, synthetic construct
  misc_feature 1..306: note = DnaE Intein-N polynucleotide sequence
  source 1..306: mol_type = other DNA
tgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaat

SEQ ID NO: 371 - protein, 102 aa, synthetic construct
  REGION 1..102: note = DnaE Intein-N polypeptide sequence
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 372 - DNA, 108 bp, synthetic construct
  misc_feature 1..108: note = DnaE Intein-C polynucleotide sequence
  source 1..108: mol_type = other DNA
atgatcaagatagctacaaggaagtatcttggcaaacaaaacgtttatgatattggagtcgaaagagatcacaactttgctctgaagaacggattcatagcttctaat

SEQ ID NO: 373 - protein, 36 aa, synthetic construct
  REGION 1..36: note = DnaE Intein-C polypeptide sequence
MIKIATRKYLGKQNVYDIGVERDHNFALKNGFIASN

SEQ ID NO: 374 - DNA, 303 bp, synthetic construct
  misc_feature 1..303: note = Cfa-N polynucleotide sequence
  source 1..303: mol_type = other DNA
tgcctgtcttatgataccgagatacttaccgttgaatatggcttcttgcctattggaaagattgtcgaagagagaattgaatgcacagtatatactgtagacaagaatggtttcgtttacacacagcccattgctcaatggcacaatcgcggcgaacaagaagtatttgagtactgtctcgaggatggaagcatcatacgagcaactaaagatcataaattcatgaccactgacgggcagatgttgccaatagatgagatattcgagcggggcttggatctcaaacaagtggatggattgcca

SEQ ID NO: 375 - protein, 101 aa, synthetic construct
  REGION 1..101: note = Cfa-N polypeptide sequence
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 376 - DNA, 159 bp, synthetic construct
  misc_feature 1..159: note = Cfa-C polynucleotide sequence
  source 1..159: mol_type = other DNA
atgaagaggactgccgatggatcagagtttgaatctcccaagaagaagaggaaagtaaagataatatctcgaaaaagtcttggtacccaaaatgtctatgatattggagtggagaaagatcacaacttccttctcaagaacggtctcgtagccagcaac

SEQ ID NO: 377 - protein, 53 aa, synthetic construct
  REGION 1..53: note = Cfa-C polypeptide sequence
MKRTADGSEFESPKKKRKVKIISRKSLGTQNVYDIGVEKDHNFLLKNGLVASN

SEQ ID NO: 378 - protein, 1081 aa, Neisseria meningitidis
  REGION 1..1081: note = MISC_FEATURE - Nme3Cas9; WP_002217928.1 type II CRISPR RNA-guided endonuclease Cas9 [Neisseria meningitidis]
  REGION 1..1081: note = MISC_FEATURE - NCBI Reference: WP_002217928.1; type II CRISPR RNA-guided endonuclease Cas9; Nme3Cas9 polypeptide sequence
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKECGHIRNQRGDYSHTFSRKDLQAELNLLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLSLEDTAFFKGLRYGKDNAEASTLMEMKAYHTISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVAYADEEDWTVIDESFRFKFVLYSNDLIKVQLKKDSFLGYFSGLDRATGAISLREHDLEKSKGKDGMHRIGVKTALSFQKYQIDEMGKEIRPCRLKKRPPVR

SEQ ID NO: 379 - RNA, 66 nt, synthetic construct
  misc_feature 1..66: note = Telomerase Ku Binding Motif oligonucleotide sequence
  source 1..66: mol_type = other RNA
ttcttgtcgtacttatagatcgctacgttatttcaattttgaaaatctgagtcctgggagtgcgga

SEQ ID NO: 380 - protein, 1094 aa, synthetic construct
  REGION 1..1094: note = Telomerase Ku Heterodimer polypeptide sequence
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQDMVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 381 - RNA, 10 nt, synthetic construct
  misc_feature 1..10: note = Telomerase Sm7 consensus site(single stranded) oligonucleotide sequence
  source 1..10: mol_type = other RNA
aatttttgga

SEQ ID NO: 382 - protein, 83 aa, synthetic construct
  REGION 1..83: note = MS2 coat protein polypeptide sequence
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 383 - RNA, 26 nt, synthetic construct
  misc_feature 1..26: note = PP7 phage operator stem loop oligonucleotide sequence
  source 1..26: mol_type = other RNA
ataaggagtttatatggaaaccctta

SEQ ID NO: 384 - protein, 128 aa, synthetic construct
  REGION 1..128: note = PP7 coat protein (PCP)polypeptide sequence
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 385 - RNA, 19 nt, synthetic construct
  misc_feature 1..19: note = SfMu Com stem loop oligonucleotide sequence
  source 1..19: mol_type = other RNA
ctgaatgcctgcgagcatc

SEQ ID NO: 386 - protein, 62 aa, synthetic construct
  REGION 1..62: note = SfMu Com binding protein polypeptide sequence
MKSIRCKNCNKLLFKADSFDHIEIRCPRCKRHIIMLNACEHPTEKHCGKREKITHSDETVRY

SEQ ID NO: 387 - protein, 47 aa, synthetic construct
  REGION 1..47: note = leucine zipper polypeptide sequence
LEIRAAFLRQRNTALRTEVAELEQEVQRLENEVSQYETRYGPLGGGK

SEQ ID NO: 388 - protein, 47 aa, synthetic construct
  REGION 1..47: note = leucine zipper polypeptide sequence
LEIEAAFLERENTALETRVAELRQRVQRLRNRVSQYRTRYGPLGGGK

SEQ ID NO: 389 - DNA, 303 bp, synthetic construct
  source 1..303: mol_type = other DNA
tgcctgagctacgataccgagatcctgaccgtggaatacggcttcctgcctatcggcaagatcgtcgaggaacggatcgagtgcacagtgtacaccgtggataagaatggcttcgtgtacacccagcctatcgctcagtggcacaacagaggcgagcaagaggtgttcgagtactgcctggaagatggcagcatcatccgggccaccaaggaccacaagtttatgaccaccgacggccagatgctgcccatcgacgagatctttgagagaggcctggacctgaaacaggtggacggactgcct

SEQ ID NO: 390 - protein, 101 aa, synthetic construct; identical to SEQ ID NO: 375

SEQ ID NO: 391 - DNA, 264 bp, synthetic construct
  source 1..264: mol_type = other DNA
tgtctggacctcaagacccaagtgcagacacctcagggcatgaaagagattagcaatatccaggtgggcgacctggtcctgagcaacaccggctacaacgaggtgctgaacgtgttccctaagtccaagaagaaatcttataagatcaccctggaagatggcaaggaaatcatctgcagcgaggaacacctgttccccacccagaccggcgagatgaacatcagcggcggactgaaggagggcatgtgcctgtacgtgaaggag

SEQ ID NO: 392 - protein, 88 aa, synthetic construct
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 393 - DNA, 267 bp, synthetic construct
  source 1..267: mol_type = other DNA
tgcctgagcctggacaccatggtggtgacaaacggcaaggccatcgagatcagagatgtgaaggtgggagattggctggaaagcgaatgtggcccagtgcaggttacagaggtgctgcctatcatcaagcagcctgtctttgagattgtgctgaaaagcggaaaaaagatccgggtgtccgctaatcacaagttccccaccaaggacggcctcaagaccatcaacagcggcctgaaggtgggcgacttcctgagaagcagagccaag

SEQ ID NO: 394 - protein, 89 aa, synthetic construct
CLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 395 - DNA, 303 bp, synthetic construct
  source 1..303: mol_type = other DNA
tgttttgtgcctggcaccctggtgaacacagagaatggcctgaagaaaatcgaggaaatcaaggtgggcgacaaggtgttcagccatacaggcaagctgcaggaggtggtggacaccctgatcttcgaccgggacgaggaaatcatctctatcaacggcattgattgcaccaagaaccacgagttctacgtgatcgataaggaaaacgctaatagagtgaacgaggacaacatccacctcttcgccagatgggtccacgccgaggaactggatatgaaaaagcacctgctgatcgagctggaa

SEQ ID NO: 396 - protein, 101 aa, synthetic construct
CFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 397 - DNA, 315 bp, synthetic construct
  source 1..315: mol_type = other DNA
tgcctggtgggctctagcgagattatcacaagaaactacggcaagaccaccatcaaggaagtggtcgagatcttcgacaacgacaagaatatccaggtgctggccttcaacacccacaccgataatatcgagtgggcccctatcaaggccgctcagctgaccagacctaacgccgagctggttgaactggaaatcgacaccctgcacggcgtgaaaacaatccggtgcacccctgaccaccccgtgtacaccaagaacagaggctacgtgcgggccgacgagctgacagatgatgacgagctcgtggtggctatc

SEQ ID NO: 398 - protein, 105 aa, synthetic construct
CLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 399 - DNA, 306 bp, synthetic construct
  source 1..306: mol_type = other DNA
tgcctgagctacgagacagagatcctgaccgtggaatatggcctgctgccaatcggaaagatcgtggaaaagcggatcgagtgcaccgtctacagcgtggacaacaacggaaatatctatacacagcctgtggcccaatggcacgaccggggcgaacaggaggtgtttgagtactgcctggaagatggttctctgattagagccaccaaggaccacaagttcatgaccgtcgacggccagatgctgcccatcgacgaaatcttcgagcgggaactcgacctgatgagagtggataacctgcccaat

SEQ ID NO: 400 - protein, 102 aa, synthetic construct; identical to SEQ ID NO: 371

SEQ ID NO: 401 - protein, 101 aa, synthetic construct; identical to SEQ ID NO: 375

SEQ ID NO: 402 - protein, 102 aa, synthetic construct; identical to SEQ ID NO: 371

SEQ ID NO: 403 - DNA, 279 bp, synthetic construct
  source 1..279: mol_type = other DNA
acaagaagcggatactgtctggacctcaagacccaagtgcagacacctcagggcatgaaagagattagcaatatccaggtgggcgacctggtcctgagcaacaccggctacaacgaggtgctgaacgtgttccctaagtccaagaagaaatcttataagatcaccctggaagatggcaaggaaatcatctgcagcgaggaacacctgttccccacccagaccggcgagatgaacatcagcggcggactgaaggagggcatgtgcctgtacgtgaaggag

SEQ ID NO: 404 - protein, 93 aa, synthetic construct
TRSGYCLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 405 - DNA, 282 bp, synthetic construct
  source 1..282: mol_type = other DNA
tctcagctgaaccggtgcctgagcctggacaccatggtggtgacaaacggcaaggccatcgagatcagagatgtgaaggtgggagattggctggaaagcgaatgtggcccagtgcaggttacagaggtgctgcctatcatcaagcagcctgtctttgagattgtgctgaaaagcggaaaaaagatccgggtgtccgctaatcacaagttccccaccaaggacggcctcaagaccatcaacagcggcctgaaggtgggcgacttcctgagaagcagagccaag

SEQ ID NO: 406 - protein, 94 aa, synthetic construct
SQLNRCLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 407 - DNA, 318 bp, synthetic construct
  source 1..318: mol_type = other DNA
ggcatcggcggaggatgttttgtgcctggcaccctggtgaacacagagaatggcctgaagaaaatcgaggaaatcaaggtgggcgacaaggtgttcagccatacaggcaagctgcaggaggtggtggacaccctgatcttcgaccgggacgaggaaatcatctctatcaacggcattgattgcaccaagaaccacgagttctacgtgatcgataaggaaaacgctaatagagtgaacgaggacaacatccacctcttcgccagatgggtccacgccgaggaactggatatgaaaaagcacctgctgatcgagctggaa

SEQ ID NO: 408 - protein, 106 aa, synthetic construct
GIGGGCFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 409 - DNA, 330 bp, synthetic construct
  source 1..330: mol_type = other DNA
ggaacaaacccatgttgcctggtgggctctagcgagattatcacaagaaactacggcaagaccaccatcaaggaagtggtcgagatcttcgacaacgacaagaatatccaggtgctggccttcaacacccacaccgataatatcgagtgggcccctatcaaggccgctcagctgaccagacctaacgccgagctggttgaactggaaatcgacaccctgcacggcgtgaaaacaatccggtgcacccctgaccaccccgtgtacaccaagaacagaggctacgtgcgggccgacgagctgacagatgatgacgagctcgtggtggctatc

SEQ ID NO: 410 - protein, 110 aa, synthetic construct
GTNPCCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 411 - DNA, 105 bp, synthetic construct
  source 1..105: mol_type = other DNA
gtcaagatcatcagcagaaagagcctgggcacccagaacgtgtacgatatcggagtgggcgagccccacaactttctgctcaagaatggcctggtggccagcaac

SEQ ID NO: 412 - protein, 35 aa, synthetic construct
VKIISRKSLGTQNVYDIGVGEPHNFLLKNGLVASN

SEQ ID NO: 413 - DNA, 114 bp, synthetic construct
  source 1..114: mol_type = other DNA
atgatgctgaaaaagatcctgaagatcgaggaactggatgagagagagctgatcgacatcgaagtgtctggcaatcacctgttctacgccaacgacatcctgacccacaacagc

SEQ ID NO: 414 - protein, 38 aa, synthetic construct
MMLKKILKIEELDERELIDIEVSGNHLFYANDILTHNS

SEQ ID NO: 415 - DNA, 138 bp, synthetic construct
  source 1..138: mol_type = other DNA
atgtgcgaaatcttcgagaacgagattgattgggacgaaatcgcctctatcgagtacgtgggcgtggaagagacaatcgacatcaacgtgaccaacgacagactgtttttcgccaatggcatcctgacccacaacagc

SEQ ID NO: 416 - protein, 46 aa, synthetic construct
MCEIFENEIDWDEIASIEYVGVEETIDINVTNDRLFFANGILTHNS

SEQ ID NO: 417 - DNA, 123 bp, synthetic construct
  source 1..123: mol_type = other DNA
atgaaattcaagctgaaggaaatcaccagcatcgagacaaagcactacaagggcaaggtgcacgatctgaccgtgaaccaggaccacagctacaacgtcagaggcaccgtggtgcataattct

SEQ ID NO: 418 - protein, 41 aa, synthetic construct
MKFKLKEITSIETKHYKGKVHDLTVNQDHSYNVRGTVVHNS

SEQ ID NO: 419 - DNA, 123 bp, synthetic construct
  source 1..123: mol_type = other DNA
atggaagccaagacctacatcggcaagctgaaatctagaaagatcgtgtccaacgaggatacatacgacatccagaccagcacccacaatttcttcgccaacgacatcctggtgcacaacagc

SEQ ID NO: 420 - protein, 41 aa, synthetic construct
MEAKTYIGKLKSRKIVSNEDTYDIQTSTHNFFANDILVHNS

SEQ ID NO: 421 - DNA, 108 bp, synthetic construct
  source 1..108: mol_type = other DNA
atgatcaagatcgccacaagaaagtacctgggcaagcagaacgtgtacgacatcggcgtggagagagaccacaacttcgccctgaagaacggctttatcgcctctaat

SEQ ID NO: 422 - protein, 36 aa, synthetic construct; identical to SEQ ID NO: 373

SEQ ID NO: 423 - protein, 36 aa, synthetic construct
MVKIISRKSLGTQNVYDIGVGEPHNFLLKNGLVASN

SEQ ID NO: 424 - protein, 35 aa, synthetic construct
IKIATRKYLGKQNVYDIGVERDHNFALKNGFIASN

SEQ ID NO: 425 - DNA, 88 bp, synthetic construct
  source 1..88: mol_type = other DNA; note = SaCas9 scaffold sequence
gttttagtactctgtaatgaaaattacagaatctactaaaacaaggcaaaatgccgtgtttatctcgtcaacttgttggcgagatttt

SEQ ID NO: 426 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 427 - DNA, 3921 bp, Homo sapiens
  source 1..3921: mol_type = other DNA
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 428 - protein, 444 aa, synthetic construct
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 429 - protein, 218 aa, synthetic construct
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 430 - protein, 8 aa, synthetic construct
GFDFSRYW

SEQ ID NO: 431 - protein, 8 aa, synthetic construct
INPTSSTI

SEQ ID NO: 432 - protein, 14 aa, synthetic construct
ARGNYYRYGDAMDY

SEQ ID NO: 433 - protein, 11 aa, synthetic construct
KSVSTSGYSYL

SEQ ID NO: 434 - protein, 9 aa, synthetic construct
QHSRELPFT

SEQ ID NO: 435 - protein, 25 aa, synthetic construct
EVKLLESGGGLVQPGGSLKLSCAAS

SEQ ID NO: 436 - protein, 17 aa, synthetic construct
MSWVRQAPGKGLEWIGE

SEQ ID NO: 437 - protein, 38 aa, synthetic construct
NFTPSLKDKVFISRDNAKNTLYLQMSKVRSEDTALYYC

SEQ ID NO: 438 - protein, 11 aa, synthetic construct
WGQGTSVTVSS

SEQ ID NO: 439 - protein, 26 aa, synthetic construct
DIALTQSPASLAVSLGQRATISCRAS

SEQ ID NO: 440 - protein, 16 aa, synthetic construct
HWYQQKPGQPPKLLIY

SEQ ID NO: 441 - protein, 36 aa, synthetic construct
NLESGVPARFSGSGSGTDFTLNIHPVEEEDAATYYC

SEQ ID NO: 442 - protein, 12 aa, synthetic construct
FGSGTKLEIKRA

SEQ ID NO: 443 - protein, 1380 aa, synthetic construct
MKKPYSIGLDIGTNSVGWAVITDDYKVPAKKMKVLGNTDRSHIKKNLIGALLFDAGNTAEDRRLKRTARRRYTRRRNRILYLQEIFAEEMNKIDESFFHRLDDSFLVPEDKRGSKYPIFATLQEEKEYHKQFPTIYHLRKQLAESNEKADLRLVYLALAHMIKYRGHFLIDDPKFKVQNNDIQGLFEKFVEEYDNVQETSLSKIKLNVTEILTAKIPKSEKQEQLLKNYPSEKKNTLFGNLIGLALGLTPNFKTNFSLENDAKLQISSESYEEDLGSLLALIGENFIELFSAVKNLSDGILLAGIVSDESPHAPLSTKMVIRFKEHEEDLAALKHFIKANLPEKYDEVFSDDSKNGYAGYVGVDSKVRKRNGKLATEEEFYKYLKDILNNVKGADYFLEKIKREDLLRKQRTFDNGTIPYQVHLEEMKAILQNQGEYYPFLKENKEKIQQILTFRIPYYVGPLARGNRDFAWLTRNSDQAIRPWNFEEVVDKASSAEDFINKMTNYDLYLPEEKVLPKHSLLYETFAVYNELTKVKFIAEGLRDYQFLDSGQKKQIVNQLFKEKRKVTEKDIIHYLHNVDGYDGIELKGIEKQFNASLSTYHDLLKIIKDKEFMDDPKNEEILENIVHTLTIFEDREMIKQRLAQYDSLFDEKVIKALTRRHYTGWGKLSAKLINGICDKQTNKTILDFLIDDDKINRNFMQLINDDGLSFKDIIQKAQVVGKIDDVKQVVQELPGSPAIKKGILQSIKIVDELVKVMGHAPESIVIEMARENQTTARGKKNSQQRYKRIEDALKNLAPGLDSNILKENPTDNIQLQNDRLFLYYLQNGKDMYTGEALDINQLSNYDIDHIVPQAFIKDDSLDNRVLTSSKDNRGKSDNVPSIEVVQKRKAFWQQLLDSKLISERKFNNLTKAERGGLDERDKVGFIKRQLVETRQITKHVAQILDARFNTEVNEKNQKIRKVKIITLKSNLVSNFRKEFGLYKVREINDYHHAHDAYLNAVVAKAILKKYPKLEPEFVYGDYQKYDLKRYISRSKDPKEIEKATEKYFFYSNLLNFFKEEVHYADGTIIKRENIEYSKDTGEIAWNKEKDFATVRKVLSCPQVNIVKKTEVQTGGFSKESILPKRNSDKLIARKKDWDPKKYGGFDSPTVAYSVLVVAKVEKGKSKKLKSVKELVGITIMERSSFEKDPVDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 444 - DNA, 4140 bp, synthetic construct
  source 1..4140: mol_type = other DNA
atgaagaagccttattccatcggcttggacatcgggactaactccgtcggatgggctgtgattaccgacgactacaaggttcccgcaaaaaagatgaaagtgctcggcaatacagatcggtcacatattaagaagaacctgattggcgctctgcttttcgatgcaggtaatactgccgaggataggcgcctcaagagaacagcccggcgtagatacactcgcagaaggaaccggattctgtacctgcaagaaatttttgctgaagaaatgaataagatcgatgagtcattcttccaccgactggacgatagtttcctggtacctgaagacaaaaggggatccaagtaccccatttttgccacactccaagaagagaaggagtaccacaagcaatttccgaccatctaccaccttaggaagcagctggcagagtccaacgaaaaggcagacttgcggttagtctacctggctcttgcacacatgataaagtaccggggacatttcttaattgacgaccctaagtttaaagttcagaataacgatattcaaggcctcttcgaaaagtttgttgaggaatatgataacgtacaagaaacctctttgtctaaaattaagctcaatgtgactgagatcctcacagcaaagattcccaaaagcgagaaacaagagcaactcctgaagaactaccctagtgagaaaaagaacaccctttttggcaatctgatcgggctcgccctgggcctgacacctaacttcaaaacaaatttctccctggagaacgacgccaagctgcaaatctcatccgagagttacgaggaagacttaggatctctcctcgcactgattggagaaaacttcattgaactcttctccgccgtgaaaaatctgagtgacggcatccttctggccggaatcgtctctgacgagagtccccacgcgccgctgagcacaaagatggtcatccggttcaaagagcacgaagaagatctcgcggcactcaaacatttcatcaaagccaacttgccagaaaaatacgatgaagtattctccgacgatagcaaaaacggctatgcgggatacgtaggagtcgacagcaaggtgcggaagcgcaatggtaagcttgctacagaagaagaattttacaagtaccttaaagatatcctcaataatgttaagggggccgactacttcctcgaaaagatcaaaagggaggacctgttgaggaaacagcgtacctttgacaacggcacaatcccgtaccaggtgcacctcgaagaaatgaaagccattctgcagaaccagggggaatactatccattcctcaaggaaaacaaggaaaaaatccagcagatccttacgtttaggattccttactatgtgggccctttggcaaggggtaaccgggatttcgcttggctcaccaggaatagtgaccaggccataaggccctggaactttgaagaagtcgtcgacaaggcctcttccgccgaagacttcattaacaaaatgactaactacgatctctacctgccagaggaaaaagtccttccaaaacatagcctgctttacgaaaccttcgccgtatacaacgagcttacaaaagtcaagttcatcgcagagggtctcagggattaccaattccttgactctggccaaaaaaagcagatcgtgaatcaacttttcaaggagaaacgaaaggtgactgaaaaggacatcattcactacctgcacaacgtcgatggatacgacgggattgaactgaagggcatagaaaagcagttcaatgctagtttgtccacgtaccacgatctgcttaaaatcattaaggataaggaattcatggatgaccctaagaacgaagagattttggaaaacatcgtccacaccctgaccattttcgaggatagggagatgataaagcaacggctggcacagtacgactcactttttgacgagaaggtgataaaagcccttacaagaaggcattataccgggtggggcaagctctccgctaagctgatcaatggtatttgcgacaagcagaccaataagactattttggacttcctgattgacgatgataagataaaccgaaactttatgcagctgattaatgatgatggactgagctttaaagatatcattcagaaggcccaggtcgtcggcaagatcgacgacgtcaaacaggtggtgcaggaacttccaggatccccggccattaagaagggaatattacagtccatcaaaattgtggatgagctggtgaaagtcatgggacatgcacctgagtctatcgtcatagagatggctcgtgaaaaccagacgacagcccgaggaaagaagaacagccaacaacggtataaacggatcgaggatgcacttaagaatctggccccggggctggattctaatatcttaaaagaaaatccaaccgataatattcaacttcagaacgaccggctttttctgtattaccttcagaatggaaaagatatgtataccggagaagccctggatataaatcagctgagtaactatgatatcgaccacattgtgcctcaggctttcattaaggatgacagcctggacaatcgcgtactcacatcctccaaggacaacagaggcaagagcgataacgtccctagcatcgaagtggtacaaaagcgtaaggccttctggcagcagctcttagactctaaattgataagcgaacggaaattcaataacctcacaaaggccgagcgtggtggcttggacgagagagacaaggtgggcttcattaaacggcagctcgtggaaaccagacagataaccaagcacgttgcccagatcctcgatgccaggttcaacaccgaagttaacgagaaaaaccaaaagattcggaaagtgaagatcatcactctcaagtctaatttggtgtccaactttagaaaagagtttggactctataaggtccgagagattaatgattatcaccacgctcacgatgcctacctcaacgcagtggtggccaaggccatcctgaaaaaatatcctaagctcgagcctgagttcgtctatggtgactatcagaagtacgacctcaagcgctatattagtaggagcaaggatcctaaagagatcgaaaaggccacagaaaagtacttcttttacagcaacttactcaacttcttcaaagaagaagtccactacgctgatggcaccatcatcaagcgagaaaatatagagtacagtaaggacacaggcgaaatcgcctggaataaagagaaggatttcgctactgtgcgaaaagttctgtcttgcccccaggtgaacattgtgaaaaagactgaggtgcagactggcgggtttagcaaagagagtatcctgccaaagcggaatagcgataagttaattgctcggaaaaaggactgggatccaaagaagtacggcggcttcgattccccaaccgttgcctacagcgtcctcgtcgtcgccaaggtggagaagggtaaaagcaagaagctgaaaagtgtaaaggaactggtcggcattaccattatggagcggagttccttcgaaaaggatccagttgattttttagaagccaaggggtataaggaggtgcgcaaggatttaattattaaactccccaagtacagcctgtttgagctggagaacggaaggaagaggatgctggctagcgccggcgagctccaaaagggcaatgagctcgccctgccaagcaagtacgtgaactttctctatctggctagccactacgagaagctgaagggatcacctgaagacaatgagcagaaacagctgttcgtcgagcagcataaacactacctcgacgaaattattgagcaaattagtgagttctcaaagcgggtaatcctcgcagatgcaaatctggataaagtgttaagcgcgtataataagcaccgggacaaaccaatccgggagcaagccgagaacatcattcatctcttcacactgaccaatcttggggctcctgcagcttttaaatatttcgatacaaccatcgacaggaagcgatatacttctacaaaggaagtgcttgatgcaactctcatccatcaaagcatcacggggctgtatgaaacccggattgacctgagtcagctgggtggggac

SEQ ID NO: 445 - RNA, 86 nt, synthetic construct
  source 1..86: mol_type = other RNA
gttttagagctgtgttgaaaaacacagcaagttaaaataaggctttgtccgtatccaacttgaaaaagtgagcaccgattcggtgc

SEQ ID NO: 446 - RNA, 78 nt, synthetic construct
  source 1..78: mol_type = other RNA
gttttagagctggaaacagcaagttaaaataaggctttgtccgtatccaacttgaaaaagtgagcaccgattcggtgc

SEQ ID NO: 447 - RNA, 76 nt, synthetic construct; identical to SEQ ID NO: 319
  source 1..76: mol_type = other RNA

SEQ ID NO: 448 - RNA, 20 nt, synthetic construct
  source 1..20: mol_type = other RNA
gctaaatgttaatgagaatg

SEQ ID NO: 449 - RNA, 20 nt, synthetic construct
  source 1..20: mol_type = other RNA
gttaatgagaatgtggaatg

SEQ ID NO: 450 - RNA, 20 nt, synthetic construct
  source 1..20: mol_type = other RNA
ttaatgagaatgtggaatgt

SEQ ID NO: 451 - RNA, 22 nt, synthetic construct
  source 1..22: mol_type = other RNA
atgagaatgtggaatgtggaaa

SEQ ID NO: 452 - RNA, 20 nt, synthetic construct
  source 1..20: mol_type = other RNA
tggaatgtggaaacaatact

SEQ ID NO: 453 - RNA, 20 nt, synthetic construct
  source 1..20: mol_type = other RNA
tggaatgtggaaacaatact

SEQ ID NO: 454 - RNA, 20 nt, synthetic construct
  source 1..20: mol_type = other RNA
aacaatacttgcacaaacaa

SEQ ID NO: 455 - RNA, 20 nt, synthetic construct
  source 1..20: mol_type = other RNA
caatacttgcacaaacaatg

SEQ ID NO: 456 - RNA, 22 nt, synthetic construct
  source 1..22: mol_type = other RNA
cacaaacaatgaggtgcataac

SEQ ID NO: 457 - RNA, 20 nt, synthetic construct
  source 1..20: mol_type = other RNA
tacttgcacaaacaatgagg

SEQ ID NO: 458 - RNA, 20 nt, synthetic construct
  source 1..20: mol_type = other RNA
tacttgcacaaacaatgagg

SEQ ID NO: 459 - RNA, 20 nt, synthetic construct
  source 1..20: mol_type = other RNA
cataattcatgtactgctcc

SEQ ID NO: 460 - RNA, 20 nt, synthetic construct
  source 1..20: mol_type = other RNA
atatctcataattcatgtac

SEQ ID NO: 461 - RNA, 20 nt, synthetic construct
  source 1..20: mol_type = other RNA
atatctcataattcatgtac

SEQ ID NO: 462 - RNA, 22 nt, synthetic construct
  source 1..22: mol_type = other RNA
ccatatctcataattcatgtac

SEQ ID NO: 463 - DNA, 23 bp, synthetic construct
  source 1..23: mol_type = other DNA
aaaatatgcaaacatcactgtgg

SEQ ID NO: 464 - DNA, 23 bp, synthetic construct
  source 1..23: mol_type = other DNA
tgtggattacttatataacaagg

SEQ ID NO: 465 - DNA, 23 bp, synthetic construct
  source 1..23: mol_type = other DNA
gttaatgagaatgtggaatgtgg

SEQ ID NO: 466 - DNA, 23 bp, synthetic construct
  source 1..23: mol_type = other DNA
caatacttgcacaaacaatgagg

SEQ ID NO: 467 - DNA, 23 bp, synthetic construct
  source 1..23: mol_type = other DNA
tggaatgtggaaacaatacttgc

SEQ ID NO: 468 - DNA, 24 bp, synthetic construct
  source 1..24: mol_type = other DNA
aatgtggaatgtggaaacaatact

SEQ ID NO: 469 - DNA, 24 bp, synthetic construct
  source 1..24: mol_type = other DNA
tggaatgtggaaacaatacttgca

SEQ ID NO: 470 - DNA, 24 bp, synthetic construct
  source 1..24: mol_type = other DNA
gcacaaacaatgaggtgcataacc

SEQ ID NO: 471 - DNA, 24 bp, synthetic construct
  source 1..24: mol_type = other DNA
atgagaatgtggaatgtggaaaca

SEQ ID NO: 472 - DNA, 23 bp, synthetic construct
  source 1..23: mol_type = other DNA
ttaatgagaatgtggaatgtgga

SEQ ID NO: 473 - RNA, 20 nt, synthetic construct
  source 1..20: mol_type = other RNA
aaaatatgcaaacatcactg

SEQ ID NO: 474 - RNA, 20 nt, synthetic construct
  source 1..20: mol_type = other RNA
tgtggattacttatataaca

SEQ ID NO: 475 - RNA, 20 nt, synthetic construct
  source 1..20: mol_type = other RNA
gttaatgagaatgtggaatg

SEQ ID NO: 476 - RNA, 20 nt, synthetic construct
  source 1..20: mol_type = other RNA
caatacttgcacaaacaatg

SEQ ID NO: 477 - RNA, 20 nt, synthetic construct
  source 1..20: mol_type = other RNA
tggaatgtggaaacaatact

SEQ ID NO: 478 - RNA, 20 nt, synthetic construct
  source 1..20: mol_type = other RNA
aatgtggaatgtggaaacaa

SEQ ID NO: 479 - RNA, 20 nt, synthetic construct
  source 1..20: mol_type = other RNA
tggaatgtggaaacaatact

SEQ ID NO: 480 - RNA, 20 nt, synthetic construct
  source 1..20: mol_type = other RNA
gcacaaacaatgaggtgcat

SEQ ID NO: 481 - RNA, 20 nt, synthetic construct
  source 1..20: mol_type = other RNA
atgagaatgtggaatgtgga

SEQ ID NO: 482 - RNA, 20 nt, synthetic construct
  source 1..20: mol_type = other RNA
ttaatgagaatgtggaatgt

SEQ ID NO: 483 - RNA, 100 nt, synthetic construct
  source 1..100: mol_type = other RNA
aatgtggaatgtggaaacaagttttagagctagaaatagcaagttaaaataaggctagtccgttatcaacttgaaaaagtggcaccgagtcggtgctttt

SEQ ID NO: 484 - RNA, 114 nt, synthetic construct
  source 1..114: mol_type = other RNA
tggaatgtggaaacaatactgttttagagccggcggaaacgccggcaagttaaaataaggctagtccgttatcaacttggacttcggtccaagtggcaccgagtcggtgctttt

SEQ ID NO: 485 - RNA, 100 nt, synthetic construct
  source 1..100: mol_type = other RNA
tggaatgtggaaacaatactgttttagagctagaaatagcaagttaaaataaggctagtccgttatcaacttgaaaaagtggcaccgagtcggtgctttt

SEQ ID NO: 486 - RNA, 100 nt, synthetic construct
  source 1..100: mol_type = other RNA
gcacaaacaatgaggtgcatgttttagagctagaaatagcaagttaaaataaggctagtccgttatcaacttgaaaaagtggcaccgagtcggtgctttt

SEQ ID NO: 487 - RNA, 100 nt, synthetic construct
  source 1..100: mol_type = other RNA
atgagaatgtggaatgtggagttttagagctagaaatagcaagttaaaataaggctagtccgttatcaacttgaaaaagtggcaccgagtcggtgctttt

SEQ ID NO: 488 - RNA, 100 nt, synthetic construct
  source 1..100: mol_type = other RNA
ttaatgagaatgtggaatgtgttttagagctagaaatagcaagttaaaataaggctagtccgttatcaacttgaaaaagtggcaccgagtcggtgctttt

SEQ ID NO: 489 - RNA, 100 nt, synthetic construct
  source 1..100: mol_type = other RNA
gttaatgagaatgtggaatggttttagagctagaaatagcaagttaaaataaggctagtccgttatcaacttgaaaaagtggcaccgagtcggtgctttt

SEQ ID NO: 490 - RNA, 119 nt, synthetic construct
  source 1..119: mol_type = other RNA
gttctgtcttttggtcaggacaaccgtctagctataagtgctgcagggtgtgagaaactcctattgctggacgatgtctcttacgaggcattagcaccacaaacaatgaggtgcataac

SEQ ID NO: 491 - RNA, 100 nt, synthetic construct
  source 1..100: mol_type = other RNA
tacttgcacaaacaatgagggttttagagctagaaatagcaagttaaaataaggctagtccgttatcaacttgaaaaagtggcaccgagtcggtgctttt

SEQ ID NO: 492 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFYRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDEGADKRTADGSEFESPKKKRKV

SEQ ID NO: 493 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLYDATLYTTFEPCVMCAGAMIHSRIGRVVFGVRNAKTGAAGSLMDVLHHPGMNHRVEITEGILADECAALLCDFYRMPRRVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDEGADKRTADGSEFESPKKKRKV

SEQ ID NO: 494 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDEGADKRTADGSEFESPKKKRKV

SEQ ID NO: 495 - protein, 1606 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKAYRSTKEVLDATLIHQSITGLYETRIDLSQLGGDEGADKRTADGSEFESPKKKRKV

SEQ ID NO: 496 - protein, 1606 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDEGADKRTADGSEFESPKKKRKV

SEQ ID NO: 497 - protein, 1594 aa, synthetic construct
MKRTADGSEFESPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFMQPTVAYSVLVVAKVEKGKSKKLKSVKELLGITIMERSSFEKNPIDFLEAKGYKEVKKDLIIKLPKYSLFELENGRKRMLASAKFLQKGNELALPSKYVNFLYLASHYEKLKGGSSGSETPGTSESATPESSGSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSPEDNEQKQLFVEQHKHYLDEIIEQISEFSKRVILADANLDKVLSAYNKHRDKPIREQAENIIHLFTLTNLGAPRAFKYFDTTIARKEYRSTKEVLDATLIHQSITGLYETRIDLSQLGGDEGADKRTADGSEFESPKKKRKV

(19) United States
(12) Patent Application Publication
(10) Pub. No.: US 2026/0130997 A1
(43) Pub. Date: May 14, 2026

(54) METHODS AND COMPOSITIONS FOR ANTI-CD45 BASED NON-GENOTOXIC CONDITIONING

(71) Applicant: Beam Therapeutics Inc., Cambridge, MA (US)

(72) Inventors: Shawn Jennings, Cambridge, MA (US); Cristina Baricordi, Cambridge, MA (US); Qing Li, Cambridge, MA (US); Charlotte McDonagh, Cambridge, MA (US); Scott Haihua Chu, Cambridge, MA (US); Jacob Hope, Cambridge, MA (US)

(73) Assignee: Beam Therapeutics Inc., Cambridge, MA (US)

(21) Appl. No.: 19/388,302

(22) Filed: Nov. 13, 2025

Related U.S. Application Data
(63) Continuation of application No. PCT/US2024/029302, filed on May 14, 2024.
(60) Provisional application No. 63/502,335, filed on May 15, 2023.

(51) Int. Cl. A61K 40/22; A61K 39/395; A61K 40/36; A61K 40/50; C12N 9/22; C12N 15/11; C12N 15/90

(52) U.S. Cl. CPC A61K 40/22; A61K 39/3955; A61K 40/36; A61K 40/50; C12N 9/226; C12N 15/11; C12N 15/907; C12N 2310/20

(57) ABSTRACT

The disclosure compositions and methods for non-genotoxic conditioning with an anti-CD45 antibody, anti-CD45 antibody drug conjugate, or anti-CD45 chimeric antigen receptor expressing T cell. The methods involve altering a CD45 polynucleotide sequence in an HSPC using a base editor to encode a CD45 polypeptide with reduced binding to the antibody. Embodiments of the disclosure also feature compositions and methods for treating diseases and conditions by non-genotoxic conditioning with an anti-CD45 antibody, anti-CD45 antibody drug conjugate, or anti-CD45 chimeric antigen receptor expressing T cell (CAR-T) prior to, simultaneously with, or after transplant of the based edited HSPC. The compositions and methods disclosed herein advantageously can reduce or eliminate the use of genotoxic agents such as busulfan, and provide edited HSPCs that are resistant to the disclosed anti-CD45 antibody, anti-CD45 antibody drug conjugate, and anti-CD45 chimeric antigen receptor expressing T cell.

### CROSS REFERENCE TO RELATED APPLICATIONS

This application is a continuation under 35 U.S.C. § 111(a) of PCT International Patent Application No. PCT/US2024/029302, filed May 14, 2024, designating the United States and published in English, which claims priority to and the benefit of U.S. Provisional Application No. 63/502,335, filed May 15, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on May 14, 2024, is named 180802-052002PCT_SL.xml and is 952,822 bytes in size.

### BACKGROUND

Busulfan is a DNA alkylating reagent that induces bone marrow immunosuppression and is widely used for conditioning prior to allogenic hematopoietic stem cell transplantation and administration of autologous cell therapies. While busulfan is the current standard of care for patients in need of allogenic or autologous transplants and engraftment of cell therapies, the use of this potent cytotoxic agent has associated risks including genotoxicity, primary or secondary malignancy, and organ toxicities including infertility. These risks present barriers to patients who would otherwise seek treatment. Accordingly, there is a need for improved methods for conditioning prior to allogeneic hematopoietic stem cell transplantation.

### SUMMARY

As described below, the disclosure features compositions and methods for non-genotoxic conditioning with an anti-CD45 antibody, anti-CD45 antibody drug conjugate, or anti-CD45 chimeric antigen receptor expressing T cell (CAR-T), where the methods altering a cluster of differentiation 45 (Protein tyrosine phosphatase, receptor type, C also known as PTPRC) polynucleotide sequence in a hematopoietic stem or progenitor cell (HSPC) with a base editor to encode a CD45 polypeptide with reduced binding to the antibody. In some embodiments, the disclosure features compositions and methods for treating diseases and conditions by non-genotoxic conditioning with an anti-CD45 antibody, anti-CD45 antibody drug conjugate, or anti-CD45 chimeric antigen receptor expressing T cell (CAR-T) prior to, simultaneously with, or after transplant of the based edited HSPC. The compositions and methods disclosed herein advantageously can reduce or eliminate the use of genotoxic agents such as busulfan, which are associated with primary or secondary malignancy, and organ toxicities including infertility. Furthermore, the compositions and methods disclosed herein provide edited HSPCs that are resistant to the disclosed anti-CD45 antibody, anti-CD45 antibody drug conjugate, and anti-CD45 chimeric antigen receptor expressing T cell (CAR-T), and therefore can be simultaneously contacted in the patient with the conditioning agent.

In one aspect, the disclosure features a method of producing an edited hematopoietic stem or progenitor cell (HSPC) for the treatment of a disease or condition. The method involves (a) expressing in the hematopoietic stem cell or progenitor thereof a nucleobase editor polypeptide. The nucleobase editor polypeptide contains a nucleic acid programmable DNA binding protein (napDNAbp) domain and a deaminase domain. The method further involves (b) contacting the hematopoietic stem cell or progenitor thereof with a guide RNA (gRNA), or a polynucleotide encoding the gRNA. The gRNA targets a polynucleotide encoding a cluster of differentiation 45 (CD45) polypeptide. The method results in introducing a missense mutation in a CD45 polynucleotide in the cell. The missense mutation is in a portion of the polynucleotide encoding the extracellular domain and/or in fibronectin domain 1 to domain 4 of the CD45 polypeptide. The missense mutation is associated with a reduction in binding of an anti-CD45 antibody to the CD45 polypeptide expressed by the edited HSPC.

In another aspect, the disclosure features a method of conditioning a human subject having a disease or condition concurrent with or prior to a hematopoietic stem cell transplant (HSCT). The method involves (a) expressing in an hematopoietic stem cell of the subject or of a donor a nucleobase editor polypeptide containing a nucleic acid programmable DNA binding protein (napDNAbp) and a deaminase. The method further involves (b) contacting the hematopoietic stem cell or progenitor cell (HSPC) with a guide RNA (gRNA), or a polynucleotide encoding the gRNA. The gRNA targets a polynucleotide encoding a cluster of differentiation 45 (CD45) polypeptide. The method results in introducing a missense mutation in the polynucleotide and generating an edited HSPC. The missense mutation is in a portion of the polynucleotide encoding the extracellular domain and/or in fibronectin domain 1 to domain 4 of the CD45 polypeptide. The method further involves (c) administering the edited hematopoietic stem cell to the subject. The method also involves (d) administering to the subject an anti-CD45 antibody, anti-CD45 antibody drug conjugate, or anti-CD45 chimeric antigen receptor expressing T cell (CAR-T) that selectively binds a wild-type CD45 protein. The administering of step (d) is prior to, concurrent with, or following step (c). The missense mutation is associated with a reduction in binding of the anti-CD45 antibody, anti-CD45 antibody drug conjugate, or anti-CD45 chimeric antigen receptor expressing T cell (CAR-T) to a CD45 polypeptide expressed by the edited HSPC.

In another aspect, the disclosure features a base editor system containing a nucleobase editor polypeptide, or a polynucleotide encoding the nucleobase editor polypeptide. The nucleobase editor polypeptide contains a nucleic acid programmable DNA binding protein (napDNAbp) domain and a deaminase domain. The base editor system also contains a guide RNA (gRNA), or a polynucleotide encoding the gRNA. The gRNA targets a polynucleotide encoding a cluster of differentiation 45 (CD45) polypeptide. The base editor system is capable of introducing a missense mutation in a CD45 polynucleotide in a cell. The missense mutation is in a portion of the polynucleotide encoding the extracellular domain and/or in fibronectin domain 1 to domain 4 of the CD45 polypeptide. The missense mutation is associated with a reduction in binding of an anti-CD45 antibody to the CD45 polypeptide expressed by the edited HSPC.

In another aspect, the disclosure features a polynucleotide or set of polynucleotides encoding the base editor system of any aspect of the disclosure, or embodiments thereof, or a component thereof.

In another aspect, the disclosure features a vector or set of vectors containing the polynucleotide or set of polynucleotides of any aspect of the disclosure, or embodiments thereof.

In another embodiment the disclosure features an HSPC prepared by the method of any aspect of the disclosure, or embodiments thereof.

In another aspect, the disclosure features an HSPC containing the base editor system, the polynucleotide or set of polynucleotides, or the vector or set of vectors of any aspect of the disclosure, or embodiments thereof.

In another aspect, the disclosure features a pharmaceutical composition containing the base editor system, the polynucleotide or set of polynucleotides, the vector or set of vectors, or the HSPC of any aspect of the disclosure, or embodiments thereof, and a pharmaceutically acceptable excipient.

In another aspect, the disclosure features a kit suitable for use in the method of any aspect of the disclosure, or embodiments thereof, where the kit contains the base editor system, the polynucleotide or set of polynucleotides, the vector or set of vectors, the HSPC, or the pharmaceutical composition of any aspect of the disclosure, or embodiments thereof, and a container.

In another aspect, the disclosure features a method of producing an edited hematopoietic stem or progenitor cell (HSPC) for the treatment of a disease or condition. The method involves (a) expressing in the hematopoietic stem cell or progenitor thereof a nucleobase editor polypeptide. The nucleobase editor polypeptide contains an SpCas9 polypeptide having specificity for a protospacer adjacent motif selected from NRCH and NGC, where “N” is A, T, G, or C, “R” is A or G, and “H” is A, C, or T. The nucleobase editor polypeptide also contains an adenosine deaminase domain. The adenosine deaminase domain contains TadA*8.20, TadA-8e, or TadA*8.20 with the amino acid alterations S82T, Y147D, F149Y, T166I, and D167N. The method further involves (b) contacting the hematopoietic stem cell or progenitor thereof with gRNA4630 or gRNA2696. The method results in introducing a missense mutation in a CD45 polynucleotide in the cell. The missense mutation results in an E259G alteration to the CD45 polypeptide encoded by the CD45 polynucleotide.

In another aspect, the disclosure features an HSPC prepared according to the method of any aspect of the disclosure, or embodiments thereof.

In another aspect, the disclosure features a method of treating a subject having a disease or condition. The method involves a) administering to the subject the HSPC cell of any aspect of the disclosure, or embodiments thereof. The method further involves b) administering to the subject an mAb030-9 polypeptide prior to, concurrent with, or following step a).

In any aspect of the disclosure, or embodiments thereof, the missense mutation does not alter CD45 biologic activity.

In any aspect of the disclosure, or embodiments thereof, the deaminase domain contains an adenosine deaminase domain or a cytidine deaminase domain. In any aspect of the disclosure, or embodiments thereof, the adenosine deaminase domain contains TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, TadA*8.24, TadA-8e, or TadA*8.20 with the amino acid alterations S82T, Y147D, F149Y, T166I, and D167N. In any aspect of the disclosure, or embodiments thereof, the adenosine deaminase domain contains TadA*8.20, TadA-8e, or TadA*8.20 with the amino acid alterations S82T, Y147D, F149Y, T166I, and D167N. In any aspect of the disclosure, or embodiments thereof, the adenosine deaminase domain further contains a wild-type TadA.

In any aspect of the disclosure, or embodiments thereof, the adenosine deaminase domain is inserted within the napDNAbp domain. In any aspect of the disclosure, or embodiments thereof, the napDNAbp domain is an SpCas9, and the adenosine deaminase domain is inserted between amino acid positions 1247 and 1248, as numbered in SEQ ID NO: 197.

In any aspect of the disclosure, or embodiments thereof, the nucleobase editor polypeptide contains an amino acid sequence with at least about 85% identity to a sequence listed in Table 17.

In any aspect of the disclosure, or embodiments thereof, the missense mutation is selected from one or more of E256G; D238G, Y239C; E259G; E259K; E259G, N262D; I283M; I283M, H285R; I283M, H285R, N286G; K231R, Y232C; N253G, N255G; N255G, E256G; N255G, E256G, N257D; N255G, E256G, N257G; N257G; N257S; N257D; E259R; N257G, E256G; N257G, E256G, N257D; N263G, T264A; N263S, T264A; N263S, T264A, T266A; N267G, N268G; N267S; N267G; N286G; T264A; T266A; T266A, N267G; and N286D.

In any aspect of the disclosure, or embodiments thereof, the gRNA contains a spacer sequence selected from a spacer sequence provided in Table 1 or Table 2. In any aspect of the disclosure, or embodiments thereof, the gRNA contains a gRNA sequence selected from those listed in Table 2.

In any aspect of the disclosure, or embodiments thereof, the napDNAbp domain is an SpCas9. In any aspect of the disclosure, or embodiments thereof, the SpCas9 has specificity for a protospacer adjacent motif sequence that is NGC or NRCH, where “N” is A, T, G, or C, “R” is A or G, and “H” is A, C, or T.

In any aspect of the disclosure, or embodiments thereof, the disease or condition is a human disease selected from one or more of sickle cell disease, beta-thalassemia, multiple sclerosis, systemic sclerosis (sSC), systemic lupus erythematosus (SLE), rheumatoid arthritis (RA), multiple myeloma, a plasma cell disorder, acute myelogenous leukemia, non-Hodgkin lymphoma, myelodysplastic syndrome, myeloproliferative neoplasm, acute lymphoblastic leukemia, Hodgkin lymphoma, chronic myeloid leukemia, HIV, and Crohn's disease.

In any aspect of the disclosure, or embodiments thereof, the anti-CD45 antibody, anti-CD45 antibody drug conjugate, or anti-CD45 chimeric antigen receptor contains the following CDRs: VH CDR1: GFDFSRYW (SEQ ID NO: 430); VH CDR2: INPTSSTI (SEQ ID NO: 431); VH CDR3: ARGNYYRYGDAMDY (SEQ ID NO: 432); VL CDR1: KSVSTSGYSYL (SEQ ID NO: 433); VL CDR2: LAS; and VL CDR3: QHSRELPFT (SEQ ID NO: 434). In any aspect of the disclosure, or embodiments thereof, the anti-CD45 antibody or anti-CD45 antibody drug conjugate is a humanized anti-CD45 antibody or anti-CD45 antibody drug conjugate.

In any aspect of the disclosure, or embodiments thereof, the vector or set of vectors are lipid nanoparticles. In any aspect of the disclosure, or embodiments thereof, the vector or set of vectors are viral vectors. In any aspect of the disclosure, or embodiments thereof, the viral vectors are adeno-associated virus vectors, lentiviral vectors, or rabies virus vectors.

In any aspect of the disclosure, or embodiments thereof, the adenosine deaminase domain is inserted between amino acid positions 1247 and 1248 of the SpCas9 polypeptide, as numbered in SEQ ID NO: 197.

In any aspect of the disclosure, or embodiments thereof, the nucleobase editor polypeptide contains an amino acid sequence having at least about 90% identity to a sequence selected from one or more of:

1570
(SEQ ID NO: 492)
MSEVEFSHEYWMRHALTLAKRARDEREVPVGAVLVLNNRVIGEGWNRAIGLHDPTAHAEIMA
LRQGGLVMQNYRLYDATLYSTFEPCVMCAGAMIHSRIGRVVFGVRNAKTGAAGSLMDVLHHP
GMNHRVEITEGILADECAALLCRFYRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMVKRYDEHHQDLTLLKALVRQQLPEKYKEIFFDQSKNGYA
GYIDGGASQEEFYKFIKPILEKMDGTEELLVKLNREDLLRKQRTFDNGIIPHQIHLGELHAI
LRRQGDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGGHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKGNSDKLIARKKDWDPKKYGGFNSPTVAYSVLVVAKVEKGKSKKLKSVKELLGI
TIMERSSFEKNPIDFLEAKGYKEVKKDLIIKLPKYSLFELENGRKRMLASAGVLQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTINRKQYNTTKEVLDATLIRQSI
TGLYETRIDLSQLGGDEGADKRTADGSEFESPKKKRKV;
2518
(SEQ ID NO: 493)
MSEVEFSHEYWMRHALTLAKRARDEREVPVGAVLVLNNRVIGEGWNRAIGLHDPTAHAEIMA
LRQGGLVMQNYRLYDATLYTTFEPCVMCAGAMIHSRIGRVVFGVRNAKTGAAGSLMDVLHHP
GMNHRVEITEGILADECAALLCDFYRMPRRVFNAQKKAQSSINSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMVKRYDEHHQDLTLLKALVRQQLPEKYKEIFFDQSKNGYA
GYIDGGASQEEFYKFIKPILEKMDGTEELLVKLNREDLLRKQRTFDNGIIPHQIHLGELHAI
LRRQGDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGGHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKGNSDKLIARKKDWDPKKYGGFNSPTVAYSVLVVAKVEKGKSKKLKSVKELLGI
TIMERSSFEKNPIDFLEAKGYKEVKKDLIIKLPKYSLFELENGRKRMLASAGVLQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTINRKQYNTTKEVLDATLIRQSI
TGLYETRIDLSQLGGDEGADKRTADGSEFESPKKKRKV;
3626
(SEQ ID NO: 496)
MKRTADGSEFESPKKKRKVSEVEFSHEYWMRHALTLAKRARDEREVPVGAVLVLNNRVIGEG
WNRAIGLHDPTAHAEIMALRQGGLVMQNYRLYDATLYSTFEPCVMCAGAMIHSRIGRVVFGV
RNAKTGAAGSLMDVLHHPGMNHRVEITEGILADECAALLCRFFRMPRRVFNAQKKAQSSTDS
GGSSGGSSGSETPGTSESATPESSGGSSGGSDKKYSIGLAIGTNSVGWAVITDEYKVPSKKF
KVLGNTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMVKRYDEHHQDLTLLKALVRQQ
LPEKYKEIFFDQSKNGYAGYIDGGASQEEFYKFIKPILEKMDGTEELLVKLNREDLLRKQRT
FDNGIIPHQIHLGELHAILRRQGDFYPFLKDNREKIEKILTFRIPYYVGPLARGNSRFAWMT
RKSEETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLK
RLRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGGHKPENIVIEMARENQTTQKGQKN
SRERMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKV
LSMPQVNIVKKTEVQTGGFSKESILPKGNSDKLIARKKDWDPKKYGGFMQPTVAYSVLVVAK
VEKGKSKKLKSVKELLGITIMERSSFEKNPIDFLEAKGYKEVKKDLIIKLPKYSLFELENGR
KRMLASAKFLQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPRAFKYFDTTIARK
EYRSTKEVLDATLIHQSITGLYETRIDLSQLGGDEGADKRTADGSEFESPKKKRKV;
and
3167
(SEQ ID NO: 497)
MKRTADGSEFESPKKKRKVDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYADLFLA
AKNLSDAILLSDILRVNTEITKAPLSASMVKRYDEHHQDLTLLKALVRQQLPEKYKEIFFDQ
SKNGYAGYIDGGASQEEFYKFIKPILEKMDGTEELLVKLNREDLLRKQRTFDNGIIPHQIHL
GELHAILRRQGDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRLRYTGWGRLSR
KLINGIRDKQSGKTILDFLKSDGFANRNFMQLIHDDSLTFKEDIQKAQVSGQGDSLHEHIAN
LAGSPAIKKGILQTVKVVDELVKVMGGHKPENIVIEMARENQTTQKGQKNSRERMKRIEEGI
KELGSQILKEHPVENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYK
VREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKT
EVQTGGFSKESILPKGNSDKLIARKKDWDPKKYGGFMQPTVAYSVLVVAKVEKGKSKKLKSV
KELLGITIMERSSFEKNPIDFLEAKGYKEVKKDLIIKLPKYSLFELENGRKRMLASAKFLQK
GNELALPSKYVNFLYLASHYEKLKGGSSGSETPGTSESATPESSGSEVEFSHEYWMRHALTL
AKRARDEREVPVGAVLVLNNRVIGEGWNRAIGLHDPTAHAEIMALRQGGLVMQNYRLYDATL
YSTFEPCVMCAGAMIHSRIGRVVFGVRNAKTGAAGSLMDVLHHPGMNHRVEITEGILADECA
ALLCRFFRMPRRVFNAQKKAQSSTDSPEDNEQKQLFVEQHKHYLDEIIEQISEFSKRVILAD
ANLDKVLSAYNKHRDKPIREQAENIIHLFTLTNLGAPRAFKYFDTTIARKEYRSTKEVLDAT
LIHQSITGLYETRIDLSQLGGDEGADKRTADGSEFESPKKKRKV.

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “cluster of differentiation 45 (CD45) polypeptide” or “protein tyrosine phosphatase, receptor type, C (PTPRC)” is meant a protein or a functional fragment thereof having tyrosine phosphatase activity and having at least about 85% amino acid sequence identity to NCBI Reference Sequence Accession No. NP_002829.3, which is provided below, and having CD45 biological activity. An exemplary CD45 polypeptide amino acid sequence from Homo sapiens is provided below (NCBI RefSeq Accession No. NP_002829.3).

### CD45 Amino Acid Sequence [Homo Sapiens] NCBI Reference Sequence

(SEQ ID NO: 426)
MTMYLWLKLLAFGFAFLDTEVFVTGQSPTPSPTGLTTAKMPSVPLSSDPLPTHTTAFSPAST
FERENDFSETTTSLSPDNTSTQVSPDSLDNASAFNTTGVSSVQTPHLPTHADSQTPSAGTDT
QTFSGSAANAKLNPTPGSNAISDVPGERSTASTFPTDPVSPLTTTLSLAHHSSAALPARTSN
TTITANTSDAYLNASETTTLSPSGSAVISTTTIATTPSKPTCDEKYANITVDYLYNKETKLF
TAKLNVNENVECGNNTCTNNEVHNLTECKNASVSISHNSCTAPDKTLILDVPPGVEKFQLHD
CTQVEKADTTICLKWKNIETFTCDTQNITYRFQCGNMIFDNKEIKLENLEPEHEYKCDSEIL
YNNHKFTNASKIIKTDFGSPGEPQIIFCRSEAAHQGVITWNPPQRSFHNFTLCYIKETEKDC
LNLDKNLIKYDLQNLKPYTKYVLSLHAYIIAKVQRNGSAAMCHFTTKSAPPSQVWNMTVSMT
SDNSMHVKCRPPRDRNGPHERYHLEVEAGNTLVRNESHKNCDFRVKDLQYSTDYTFKAYFHN
GDYPGEPFILHHSTSYNSKALIAFLAFLIIVTSIALLVVLYKIYDLHKKRSCNLDEQQELVE
RDDEKQLMNVEPIHADILLETYKRKIADEGRLFLAEFQSIPRVFSKFPIKEARKPFNQNKNR
YVDILPYDYNRVELSEINGDAGSNYINASYIDGFKEPRKYIAAQGPRDETVDDFWRMIWEQK
ATVIVMVTRCEEGNRNKCAEYWPSMEEGTRAFGDVVVKINQHKRCPDYIIQKLNIVNKKEKA
TGREVTHIQFTSWPDHGVPEDPHLLLKLRRRVNAFSNFFSGPIVVHCSAGVGRTGTYIGIDA
MLEGLEAENKVDVYGYVVKLRRQRCLMVQVEAQYILIHQALVEYNQFGETEVNLSELHPYLH
NMKKRDPPSEPSPLEAEFQRLPSYRSWRTQHIGNQEENKSKNRNSNVIPYDYNRVPLKHELE
MSKESEHDSDESSDDDSDSEEPSKYINASFIMSYWKPEVMIAAQGPLKETIGDFWQMIFQRK
VKVIVMLTELKHGDQEICAQYWGEGKQTYGDIEVDLKDTDKSSTYTLRVFELRHSKRKDSRT
VYQYQYTNWSVEQLPAEPKELISMIQVVKQKLPQKNSSEGNKHHKSTPLLIHCRDGSQQTGI
FCALLNLLESAETEEVVDIFQVVKALRKARPGMVSTFEQYQFLYDVIASTYPAQNGQVKKNN
HQEDKIEFDNEVDKVKQDANCVNPLGAPEKLPEAKEQAEGSEPTSGTEGPEHSVNGPASPAL
NQGS.

By “CD45 polynucleotide” is meant a nucleic acid molecule encoding a CD45 polypeptide, as well as the introns, exons, and regulatory sequences associated with its expression, or fragments thereof. In embodiments, an CD45 polynucleotide is the genomic sequence, which corresponds to a Homo sapiens genomic sequence encoding a CD45 polypeptide), miRNA, or gene associated with and/or required for CD45 polypeptide expression. An exemplary CD45 gene sequence is provided at ENSEMBL accession No. ENSG00000081237. An exemplary CD45 nucleotide sequence from Homo Sapiens is provided below (NCBI RefSeq Accession No. NM_002838.5):
NM_002838.5:142-4062 Homo sapiens protein tyrosine phosphatase receptor type C (PTPRC) transcript variant 1, mRNA

(SEQ ID NO: 427)
ATGACCATGTATTTGTGGCTTAAACTCTTGGCATTTGGCTTTGCCTTTCTGGACACAGAAGT
ATTTGTGACAGGGCAAAGCCCAACACCTTCCCCCACTGGATTGACTACAGCAAAGATGCCCA
GTGTTCCACTTTCAAGTGACCCCTTACCTACTCACACCACTGCATTCTCACCCGCAAGCACC
TTTGAAAGAGAAAATGACTTCTCAGAGACCACAACTTCTCTTAGTCCAGACAATACTTCCAC
CCAAGTATCCCCGGACTCTTTGGATAATGCTAGTGCTTTTAATACCACAGGTGTTTCATCAG
TACAGACGCCTCACCTTCCCACGCACGCAGACTCGCAGACGCCCTCTGCTGGAACTGACACG
CAGACATTCAGCGGCTCCGCCGCCAATGCAAAACTCAACCCTACCCCAGGCAGCAATGCTAT
CTCAGATGTCCCAGGAGAGAGGAGTACAGCCAGCACCTTTCCTACAGACCCAGTTTCCCCAT
TGACAACCACCCTCAGCCTTGCACACCACAGCTCTGCTGCCTTACCTGCACGCACCTCCAAC
ACCACCATCACAGCGAACACCTCAGATGCCTACCTTAATGCCTCTGAAACAACCACTCTGAG
CCCTTCTGGAAGCGCTGTCATTTCAACCACAACAATAGCTACTACTCCATCTAAGCCAACAT
GTGATGAAAAATATGCAAACATCACTGTGGATTACTTATATAACAAGGAAACTAAATTATTT
ACAGCAAAGCTAAATGTTAATGAGAATGTGGAATGTGGAAACAATACTTGCACAAACAATGA
GGTGCATAACCTTACAGAATGTAAAAATGCGTCTGTTTCCATATCTCATAATTCATGTACTG
CTCCTGATAAGACATTAATATTAGATGTGCCACCAGGGGTTGAAAAGTTTCAGTTACATGAT
TGTACACAAGTTGAAAAAGCAGATACTACTATTTGTTTAAAATGGAAAAATATTGAAACCTT
TACTTGTGATACACAGAATATTACCTACAGATTTCAGTGTGGTAATATGATATTTGATAATA
AAGAAATTAAATTAGAAAACCTTGAACCCGAACATGAGTATAAGTGTGACTCAGAAATACTC
TATAATAACCACAAGTTTACTAACGCAAGTAAAATTATTAAAACAGATTTTGGGAGTCCAGG
AGAGCCTCAGATTATTTTTTGTAGAAGTGAAGCTGCACATCAAGGAGTAATTACCTGGAATC
CCCCTCAAAGATCATTTCATAATTTTACCCTCTGTTATATAAAAGAGACAGAAAAAGATTGC
CTCAATCTGGATAAAAACCTGATCAAATATGATTTGCAAAATTTAAAACCTTATACGAAATA
TGTTTTATCATTACATGCCTACATCATTGCAAAAGTGCAACGTAATGGAAGTGCTGCAATGT
GTCATTTCACAACTAAAAGTGCTCCTCCAAGCCAGGTCTGGAACATGACTGTCTCCATGACA
TCAGATAATAGTATGCATGTCAAGTGTAGGCCTCCCAGGGACCGTAATGGCCCCCATGAACG
TTACCATTTGGAAGTTGAAGCTGGAAATACTCTGGTTAGAAATGAGTCGCATAAGAATTGCG
ATTTCCGTGTAAAAGATCTTCAATATTCAACAGACTACACTTTTAAGGCCTATTTTCACAAT
GGAGACTATCCTGGAGAACCCTTTATTTTACATCATTCAACATCTTATAATTCTAAGGCACT
GATAGCATTTCTGGCATTTCTGATTATTGTGACATCAATAGCCCTGCTTGTTGTTCTCTACA
AAATCTATGATCTACATAAGAAAAGATCCTGCAATTTAGATGAACAGCAGGAGCTTGTTGAA
AGGGATGATGAAAAACAACTGATGAATGTGGAGCCAATCCATGCAGATATTTTGTTGGAAAC
TTATAAGAGGAAGATTGCTGATGAAGGAAGACTTTTTCTGGCTGAATTTCAGAGCATCCCGC
GGGTGTTCAGCAAGTTTCCTATAAAGGAAGCTCGAAAGCCCTTTAACCAGAATAAAAACCGT
TATGTTGACATTCTTCCTTATGATTATAACCGTGTTGAACTCTCTGAGATAAACGGAGATGC
AGGGTCAAACTACATAAATGCCAGCTATATTGATGGTTTCAAAGAACCCAGGAAATACATTG
CTGCACAAGGTCCCAGGGATGAAACTGTTGATGATTTCTGGAGGATGATTTGGGAACAGAAA
GCCACAGTTATTGTCATGGTCACTCGATGTGAAGAAGGAAACAGGAACAAGTGTGCAGAATA
CTGGCCGTCAATGGAAGAGGGCACTCGGGCTTTTGGAGATGTTGTTGTAAAGATCAACCAGC
ACAAAAGATGTCCAGATTACATCATTCAGAAATTGAACATTGTAAATAAAAAAGAAAAAGCA
ACTGGAAGAGAGGTGACTCACATTCAGTTCACCAGCTGGCCAGACCACGGGGTGCCTGAGGA
TCCTCACTTGCTCCTCAAACTGAGAAGGAGAGTGAATGCCTTCAGCAATTTCTTCAGTGGTC
CCATTGTGGTGCACTGCAGTGCTGGTGTTGGGCGCACAGGAACCTATATCGGAATTGATGCC
ATGCTAGAAGGCCTGGAAGCCGAGAACAAAGTGGATGTTTATGGTTATGTTGTCAAGCTAAG
GCGACAGAGATGCCTGATGGTTCAAGTAGAGGCCCAGTACATCTTGATCCATCAGGCTTTGG
TGGAATACAATCAGTTTGGAGAAACAGAAGTGAATTTGTCTGAATTACATCCATATCTACAT
AACATGAAGAAAAGGGATCCACCCAGTGAGCCGTCTCCACTAGAGGCTGAATTCCAGAGACT
TCCTTCATATAGGAGCTGGAGGACACAGCACATTGGAAATCAAGAAGAAAATAAAAGTAAAA
ACAGGAATTCTAATGTCATCCCATATGACTATAACAGAGTGCCACTTAAACATGAGCTGGAA
ATGAGTAAAGAGAGTGAGCATGATTCAGATGAATCCTCTGATGATGACAGTGATTCAGAGGA
ACCAAGCAAATACATCAATGCATCTTTTATAATGAGCTACTGGAAACCTGAAGTGATGATTG
CTGCTCAGGGACCACTGAAGGAGACCATTGGTGACTTTTGGCAGATGATCTTCCAAAGAAAA
GTCAAAGTTATTGTTATGCTGACAGAACTGAAACATGGAGACCAGGAAATCTGTGCTCAGTA
CTGGGGAGAAGGAAAGCAAACATATGGAGATATTGAAGTTGACCTGAAAGACACAGACAAAT
CTTCAACTTATACCCTTCGTGTCTTTGAACTGAGACATTCCAAGAGGAAAGACTCTCGAACT
GTGTACCAGTACCAATATACAAACTGGAGTGTGGAGCAGCTTCCTGCAGAACCCAAGGAATT
AATCTCTATGATTCAGGTCGTCAAACAAAAACTTCCCCAGAAGAATTCCTCTGAAGGGAACA
AGCATCACAAGAGTACACCTCTACTCATTCACTGCAGGGATGGATCTCAGCAAACGGGAATA
TTTTGTGCTTTGTTAAATCTCTTAGAAAGTGCGGAAACAGAAGAGGTAGTGGATATTTTTCA
AGTGGTAAAAGCTCTACGCAAAGCTAGGCCAGGCATGGTTTCCACATTCGAGCAATATCAAT
TCCTATATGACGTCATTGCCAGCACCTACCCTGCTCAGAATGGACAAGTAAAGAAAAACAAC
CATCAAGAAGATAAAATTGAATTTGATAATGAAGTGGACAAAGTAAAGCAGGATGCTAATTG
TGTTAATCCACTTGGTGCCCCAGAAAAGCTCCCTGAAGCAAAGGAACAGGCTGAAGGTTCTG
AACCCACGAGTGGCACTGAGGGGCCAGAACATTCTGTCAATGGTCCTGCAAGTCCAGCTTTA
AATCAAGGTTCATAG.

By “CD45 polypeptide biologic activity” is meant tyrosine phosphatase activity.

By “mAb U139AGL030-9 (mAb030-9) polypeptide” is meant an antibody having at least about 85% amino acid sequence identity to an antibody sequence of antibody mAb030-9 or comprising VH and/or VL CDRs 1-3 of mAb030-9 or antigen binding fragments thereof, wherein each of the antibody, CDRs, and antigen binding fragments specifically bind to a wild type CD45 polypeptide, but fail to detectably bind or have only reduced binding to an altered CD45 polypeptide. In embodiments, the antibody sequence is a variable light chain region amino acid sequence, a variable heavy chain region amino acid sequence, a heavy chain amino acid sequence, a light chain amino acid sequence, or antigen-binding fragments thereof of the mAb030-9 polypeptide. In embodiments, the antibody or antigen binding fragment thereof has at least 90%, 93%, 95%, 98%, 99% or 100% amino acid sequence identity to an antibody sequence of antibody mAb030-9. Exemplary heavy chain and light chain sequences for antibody mAb030-9 are provided below, where the variable domains are in plain text, the constant domains are in bold, and complementarity determining regions (CDRs), i.e., CDR1, CDR2, and CDR2, are underlined:

mAb030-9 Heavy chain
(SEQ ID NO: 428)
EVKLLESGGGLVQPGGSLKLSCAASGFDFSRYWMSWVRQAPGKGLEWIGEINPTSSTINFTP
SLKDKVFISRDNAKNTLYLQMSKVRSEDTALYYCARGNYYRYGDAMDYWGQGTSVTVSSAKT
TPPSVYPLAPGSAAQTNSMVTLGCLVKGYFPEPVTVTWNSGSLSSGVHTFPAVLQSDLYTLS
SSVTVPSSTWPSETVTCNVAHPASSTKVDKKIVPRDCGCKPCICTVPEVSSVFIFPPKPKDV
LTITLTPKVTCVVVDISKDDPEVQFSWFVDDVEVHTAQTQPREEQFNSTFRVSELPIMHQDW
LNGKEFKCRVNSAAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTCSVLHEGLHNHHT
EKSLSHSPGK
mAb030-9 Light chain
(SEQ ID NO: 429)
DIALTQSPASLAVSLGQRATISCRASKSVSTSGYSYLHWYQQKPGQPPKLLIYLASNLESGV
PARFSGSGSGTDFTLNIHPVEEEDAATYYCQHSRELPFTFGSGTKLEIKRADAAPTVSIFPP
SSEQLTSGGASVVCFLNNFYPKDINVKWKIDGSERQNGVLNSWTDQDSKDSTYSMSSTLTLT
KDEYERHNSYTCEATHKTSTSPIVKSFNRNEC.

The three CDRs of the mAb030-9 antibody VH region are as follows:
VH CDR1: GFDFSRYW (SEQ ID NO: 430);
VH CDR2: INPTSSTI (SEQ ID NO: 431); and
VH CDR3: ARGNYYRYGDAMDY (SEQ ID NO: 432).

The three CDRs of the mAb030-9 antibody VL region are as follows:
VL CDR1: KSVSTSGYSYL (SEQ ID NO: 433);
VL CDR2: LAS; and
VL CDR3: QHSRELPFT (SEQ ID NO: 434).

The four framework (FR) regions, i.e., FR1, FR2, FR3, and FR4, of the mAb030-9 antibody are located on either side of each of the CDRs in VH and VL region sequences shown supra. In particular, the four FRs of the mAb030-9 antibody VH region are as follows:

VH FR1:
(SEQ ID NO: 435)
EVKLLESGGGLVQPGGSLKLSCAAS;
VH FR2:
(SEQ ID NO: 436)
MSWVRQAPGKGLEWIGE;
VH FR3:
(SEQ ID NO: 437)
NFTPSLKDKVFISRDNAKNTLYLQMSKVRSEDTALYYC;
and
VH FR4:
(SEQ ID NO: 438)
WGQGTSVTVSS.

The four FRs of the mAb030-9 antibody VL region are as follows:

VH FR1:
(SEQ ID NO: 439)
DIALTQSPASLAVSLGQRATISCRAS;
VH FR2:
(SEQ ID NO: 440)
HWYQQKPGQPPKLLIY;
VH FR3:
(SEQ ID NO: 441)
NLESGVPARFSGSGSGTDFTLNIHPVEEEDAATYYC;
and
VH FR4:
(SEQ ID NO: 442)
FGSGTKLEIKRA.

By “mAb U139AGL030-9 (mAb030-9) polynucleotide” is meant a nucleic acid molecule (e.g., DNA) encoding at least a fragment of a mAb030-9 antibody. In an embodiment, the encoded fragment has antigen binding activity.

By “adenine” or “9H-Purin-6-amine” is meant a purine nucleobase with the molecular formula C₅H₅N₅, having the structure

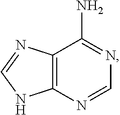

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

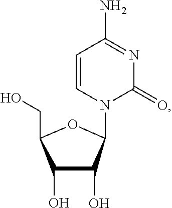

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence:

(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD,
or a

corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof. Non-limiting examples of agents suitable for use in the methods of the present disclosure include antibody-drug conjugates (ADCs).

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduce in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)2, Fab, Fv, rlgG, and scFv fragments. Further non-limiting examples of antibodies include VHH domains. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as CD45 in the context of four framework (FR) regions, as follows: FR1-CDR1-FR2-CDR2-FR3-CDR3-FR4. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding.

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C.

By “base editing efficiency” is meant the total percent of one or more target bases in a sample that have been modified using a base editor. In some cases, the base editing efficiency is calculated as the total percent of target polynucleotides in a sample containing a modified target base. In some instances, the base editing efficiency is calculated as the total percent of target polynucleotides in a sample containing a modification to one or more (e.g., 2, 3, 4, 5, 6, 7, 8, 9 or 10) of 2, 3, 4, 5, 6, 7, 8, 9, or 10 target bases. Methods for measuring base editing efficiency for a base editor are known in the art (see, e.g., Gaudelli, et al. Nature 551:464-471 (2017), the disclosure of which is incorporated herein in its entirety for all purposes). In some cases a base editing efficiency is a median base editing efficiency calculated across 2, 3, 4, 5, 6, 7, 8, 9, 10, 15, 20, 25, 30, 35, 40, 45, 50, 60, 70, 80, 90, 100 or more target sites.

By “base editing window” for a base editor is meant bases within a target polynucleotide sequence that can be modified using the base editor. In some embodiments, the position of the nucleobases in the target polynucleotide sequence are numbered relative to a protospacer adjacent motif (PAM) for which a nucleic acid programmable DNA binding protein (napDNAbp) domain of the base editor has specificity, where base 1 corresponds to the base immediately adjacent to the PAM. In some embodiments, the position of the nucleobases in the target polynucleotide sequence are numbered relative to the 5′ or 3′ end of a spacer of a guide polynucleotide used to guide a nucleic acid programmable DNA binding protein (napDNAbp) domain of the base editor to a target site, where base 1 corresponds to the 5′ or 3′ terminal base of the spacer.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain joined to one or more intracellular signaling domains (e.g., T cell signaling domain) that confers specificity for an antigen onto an immune effector cell (e.g., a T-cell, an NK cell, or a macrophage). In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” includes T cells, regulatory T cells (T_{REG}), macrophages, or NK cells. As used herein, “CAR-T cells” include cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

By “chimeric antigen receptor” or “CAR” is meant a synthetic or engineered receptor comprising an extracellular antigen binding domain operationally joined to one or more intracellular signaling domains where the CAR confers specificity for an antigen bound by the extracellular antigen binding domain onto an immune effector cell. In some cases, the intracellular signaling domain is a T cell signaling domain. In embodiments, the immune effector cell is a T cell, an NK cell, or a macrophage. In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” includes T cells, regulatory T cells (TUG), macrophages, or NK cells. As used herein, the term “CAR-T cells” includes cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

As used herein, the terms “condition” and “conditioning” refer to processes by which a patient is prepared for receipt of a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for hematopoietic stem cell transplant therapy by administration to the patient of an antibody or antigen-binding fragment thereof capable of binding an antigen expressed by hematopoietic stem cells, such as CD45. Such antibodies are expected to act via complement-mediated cytotoxicity and antibody-dependent cell-mediated cytotoxicity. As described herein, the transplanted cells have been edited so that an anti-CD45 antibody no longer binds the antigen (e.g., CD45). Administration of an antibody, antigen-binding fragment thereof, antibody-drug conjugate (ADC), or chimeric antigen receptor expressing T-cell (CAR-T) capable of binding one or more antigens (e.g., CD45) to a patient in need of hematopoietic stem cell transplant therapy can promote the engraftment of a hematopoietic stem cell graft, for example, by selectively depleting endogenous hematopoietic stem cells expressing CD45, thereby creating a vacancy filled by an exogenous hematopoietic stem cell transplant.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

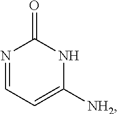

and corresponding to CAS No. 71-30-7.

By “cytidine” is meant a cytosine molecule attached to a ribose sugar via a glycosidic bond, having the structure

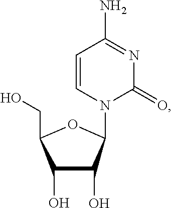

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

The term “detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include, but are not limited to, sickle cell disease, beta-thalassemia, multiple sclerosis, systemic sclerosis (sSC), systemic lupus erythematosus (SLE), rheumatoid arthritis (RA), multiple myeloma, a plasma cell disorder, acute myelogenous leukemia, non-Hodgkin lymphoma, myelodysplastic syndrome, myeloproliferative neoplasm, acute lymphoblastic leukemia, Hodgkin lymphoma, chronic myeloid leukemia, HIV, or Crohn's disease. In some embodiments, the disease is an autoimmune disease. In some embodiments, the disease is a blood cancer.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

A “framework (FR) region” or “FR region” includes amino acid residues that are adjacent to the CDRs in VH, and VL regions, and in VHHs. For example, FR region residues may be present in antibodies as described herein, camelid antibodies (VHHs), human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.”

By “heterologous,” or “exogenous” is meant a polynucleotide or polypeptide that 1) has been experimentally incorporated into a polynucleotide or polypeptide sequence to which the polynucleotide or polypeptide is not normally found in nature; and/or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a VHH or a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature. 332:323-7, 1988; Kasmiri et al., Methods, 36(1):25-34, 2005; U.S. Pat. Nos. 5,530,101; 5,585,089; 5,693,761; 5,693,762; and U.S. Pat. No. 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In an embodiment, the preparation is at least 75%, at least 90%, and at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “nick guide RNA (nickRNA; nsgRNA; ngRNA; nRNA)”, or “nicking guide RNA (nickRNA; nsgRNA; ngRNA; nRNA)”, refers to a guide polynucleotide comprising a spacer and a scaffold sequence and that is capable of guiding a nucleic acid programmable DNA binding (napDNAbp) protein domain of a prime editor to nick a target site in a target polynucleotide, where the nick guide RNA does not comprise an intended nucleotide edit for incorporation into a double stranded target polynucleotide. In some instances, the target polynucleotide is double stranded DNA.

The terms “nucleic acid,” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine).

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “OpenCRISPR-1 polypeptide” is meant a protein with an amino acid sequence having at least about 85% amino acid sequence identity to SEQ ID NO: 443, or a fragment thereof that associates with a nucleic acid, such as a guide nucleic acid or guide polynucleotide, that guides the napDNAbp to a specific nucleic acid sequence. Further details relating to the OpenCRISPR-1 polypeptide are disclosed in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes.

By “OpenCRISPR-1 polynucleotide” is meant a nucleic acid molecule encoding an OpenCRISPR-1 polypeptide, as well as the introns, exons, 3′ untranslated regions, 5′ untranslated regions, and regulatory sequences associated with its expression, or fragments thereof. In embodiments, an OpenCRISPR-1 polynucleotide is the genomic sequence, cDNA, mRNA, or gene associated with and/or required for OpenCRISPR-1 expression. An exemplary OpenCRISPR-1 nucleotide sequence is provided at SEQ ID NO: 444.

In various embodiments, a guide RNA suitable for use in combination with an OpenCRISPR-1 polypeptide contains a scaffold having at least 85% sequence identity to a nucleotide sequence selected from the following, or fragments thereof capable of binding to an OpenCRISPR-1 polypeptide:

(SEQ ID NO: 445)
GUUUUAGAGCUGUGUUGAAAAACACAGCAAGUUAAAAUAAGGCUUUGUCC
GUAUCCAACUUGAAAAAGUGAGCACCGAUUCGGUGC;
(SEQ ID NO: 446)
GUUUUAGAGCUGGAAACAGCAAGUUAAAAUAAGGCUUUGUCCGUAUCCAA
CUUGAAAAAGUGAGCACCGAUUCGGUGC;
and
(SEQ ID NO: 447)
GUUUUAGAGCUAGAAAUAGCAAGUUAAAAUAAGGCUAGUCCGUUAUCAAC
UUGAAAAAGUGGCACCGAGUCGGUGC.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

By “prime editor (PE)” is meant the polypeptide or polypeptide components involved in prime editing. In various embodiments, a prime editor comprises a polypeptide domain having DNA binding activity (e.g., a DNA binding domain) and a polypeptide domain (e.g., a DNA polymerase domain) having DNA polymerase activity. In embodiments, a prime editor contains Cas9 protein domain of S. pyogenes, or a functional fragment or variant thereof, and a reverse transcriptase domain from a retrovirus (e.g., Moloney murine leukemia virus), or a functional fragment or variant thereof. Prime editors and methods for use thereof are described in International Patent Application Publication No. WO 2023/283092, the disclosure of which is incorporated herein by reference in its entirety for all purposes.

The term “prime editing” refers to programmable editing of a target DNA using a prime editor complexed with a prime editing guide RNA (PEgRNA) to incorporate an intended nucleotide alteration into the target DNA through target-primed DNA synthesis. A target DNA polynucleotide, e.g., a target gene of prime editing can comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand can also be referred to as the “non-Protospacer Adjacent Motif (non-PAM strand).” In some embodiments, the non-target strand can also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence can be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease, e.g., a Cas9 nickase or a Cas9 nuclease. A protospacer sequence refers to a specific sequence in the PAM strand of the double stranded target DNA (e.g., target gene) that is complementary to the search target sequence. In a PEgRNA, a spacer sequence can have a substantially identical sequence as the protospacer sequence on the edit strand of the double stranded target DNA (e.g., target gene) except that the spacer sequence can comprise Uracil (U) and the protospacer sequence can comprise Thymine (T).

The term “prime editing guide RNA”, or “PEgRNA”, refers to a guide polynucleotide that comprises one or more intended nucleotide edits for incorporation into a double stranded target polynucleotide. In some instances, the target polynucleotide is double stranded DNA. In some embodiments, the PEgRNA associates with and directs a prime editor to incorporate the one or more intended nucleotide edits into the double stranded target DNA, e.g., a target gene via prime editing.

The term “prime editing system” or “prime editor system” refers to an intermolecular complex for editing a nucleobase of a target nucleotide sequence. In various embodiments, the prime editor (PE) system comprises (1) a fusion protein comprising a domain having DNA binding activity (e.g., a nucleic acid programmable DNA binding protein) and a polymerase (e.g., a DNA polymerase or a reverse transcriptase); and (2) one or more guide polynucleotides (e.g., prime editing guide RNA and/or a nick guide RNA (“nickRNA”)) in conjunction with the domain having DNA binding activity.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In some instances, a reference is an unedited cell. In some cases, a reference is a subject not administered an edited cell of the disclosure.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

By “selectively binds” is meant specifically binds a wild-type version of a CD45 polypeptide but exhibits reduced binding or fails to bind to a CD45 polypeptide comprising a mutation. In embodiments, the mutation to the CD45 polypeptide is selected from those mutations provided herein.

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:
splP14739IUNGI_BPPB2 Uracil-DNA glycosylase inhibitor

(SEQ ID NO: 231)
MTNLSDIIEKETGKQLVIQESILMLPEEVEEVIGNKPESDILVHTAYDES
TDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A and 1B provide a schematic diagram and bar graphs relating to mAb030-9-relevant edits in cell lines. In FIG. 1B, each left bar corresponds to D3 (day 3 post-electroporation (EP)) and each right bar corresponds to D7 (day 7 post-EP). In FIGS. 1A and 1B, “NGS” indicates next-generation sequencing.

FIGS. 2A to 2C provide plots and bar graphs relating to mAb030-9-relevant edits in MOLM13.

FIG. 3 provides scatter plots and a schematic diagram showing mAb030-9 antibody ESCAPE seen on Y232C and K231R/Y232C mutations. CD34 guide mutation frequency: Y232C=56.02%, K231R/Y232C=10.24%. Combined Escape Editing=66.26%. Both Y232C and K231R/Y232C showed ESCAPE from mAb030-9 and represented high combined frequency of edits in CD34+ cells.

FIG. 4 provides scatter plots and a schematic diagram showing mAb030-9 antibody ESCAPE seen on the E259G mutation. CD34 guide mutation frequency: E259G=63.6%; Silent=28.71%. 63.6% Escape Editing. E259G demonstrated ESCAPE from mAb030-9 antibody and represented a high frequency of the edits in CD34+ cells

FIG. 5 provides scatter plots and a schematic diagram showing mAb030-9 antibody ESCAPE seen on transient pools of N255G/E256G. Guide mutation frequencies: N255G=48.79%; N255D=1.03%; N255G/E256G=3.36%. 3.36% Escape Editing. N255G/E256G had some effect.

FIGS. 6A to 6C provide plots showing the kinetics of binding of mAb030-9 to CD45 polypeptides containing the indicated amino acid substitutions. In FIGS. 6A to 6C the term “PBD” indicates phosphate buffered saline, and “WT” indicates a wild-type CD45 polypeptide. The data of each of FIGS. 6A to 6C, was generated by immobilizing 100 nM of mAB030-9 to a biosensor with an Anti-Human IgG Fc Capture (AHC) antibody and contacting the immobilized mAB030-9 with 300 nM the indicated mutant or wild type CD45 protein, followed by replacing the CD45 protein containing solution with PBS at the time indicated by the dashed vertical line. CD45 polypeptides containing the amino acid substitution E259R, E259K, or N257S all showed reduced binding kinetics to mAb030-9 relative to WT CD45, indicating that the edits resulted in CD45 mutants with reduced binding affinity to mAb030-9. All CD45 variants had a response lower than 0.05 nm.

FIG. 7 provides a plot showing geometric mean fluorescence intensity for cells surface-expressing the indicated CD45 variants and immunostained using a labeled mAb030-9 polypeptide. The cells were electroporated with the base editor systems indicated along the x-axis (see Tables 10 to 15) and measurements were taken at day 7 post-electroporation. “Mock” indicates cells that were mock edited and thus express WT CD45. EP9 to EP15 exhibit reduced MFI compared to mock, indicating that the mutations reduced binding affinity to mAb030-9 and were made at a sufficient efficiency that a detectable percentage of cells had the edit.

### DETAILED DESCRIPTION

The disclosure provides a base editing strategy targeting the CD45 cell surface protein that is useful for non-toxic conditioning of a subject.

The various aspects of the disclosure are based, at least in part, on the discovery that base editing can be used to edit a CD45 polynucleotide resulting in an alteration in a CD45 polypeptide that reduces or eliminates binding of anti-CD45 antibodies, antibody drug conjugates (ADCs), or chimeric antigen receptor (CAR)-T cells to a cell comprising the edited CD45 polynucleotide. In embodiments, editing of the CD45 polynucleotide alters binding of an anti-CD45 antibody to the CD45 polypeptide, for example, by altering an epitope present on the CD45 polypeptide. In embodiments, editing of the CD45 polynucleotide reduces or eliminates CD45 expression. In some embodiments, this alteration in the CD45 polypeptide does not alter CD45 polypeptide biological activity.

In one example, an anti-CD45 antibody is used to target a CD45-expressing cell present in a subject for ablation. The ablation may be carried out before, during, or after transplantation into the subject of an HSC comprising an edited CD45 polynucleotide, such that the transplanted HSC is not targeted by the antibody. In one embodiment, the anti-CD45 antibody does not bind to or deplete CD45-edited cells. Accordingly, a cell comprising an edited CD45 polynucleotide may be expanded in vivo in the presence of a CD45 antibody (e.g., a CD45 antibody drug conjugate).

There are several advantages of the conditioning methods of the present disclosure. The methods described herein provide for the selective targeting of endogenous HSCs, while sparing edited HSCs. Accordingly, antibody or ADC treatment can continue to be administered following HSCT to expand gene edited cells in vivo. In embodiments, the base-edited HSC cells are not associated with a disease and, thus, it is advantageous to expand the base-edited HSC cells in a subject to replace pathogenic cells with the non-pathogenic base-edited HSC cells. The edited cells allow for the administration of antibody or ADC without risk for toxicity or any need to ensure that the ADC has a short serum half-life. This has the potential to enable the use of antibodies with longer half-lives, and simplify the development of ADCs. Non-limiting examples of ADCs for treatment of multiple sclerosis include those provided herein. Clinical trial design is also simplified—HSCs could be infused prior to or concurrently with conditioning with little or no risk of being depleted. The methods provide a benefit for all patients regardless of immune status.

In one embodiment, CD45 is altered (e.g., using base editing) in a cell for transplantation to prevent binding of anti-CD45 antibody, but not interfere with normal CD45 function in regulation of signal transduction in hematopoiesis. Using base editing, a nucleobase change may be generated to create an amino acid substitution in CD45. Administering an anti-CD45 antibody drug conjugate targeting a CD45 to a patient containing native cells expressing the CD45 depletes HSCs and progenitor cells in the patient (conditioning). Autologous gene edited HSCs can be transplanted into the patient. The gene-edited cells compete with residual host HSCs to repopulate bone marrow (BM). The anti-CD45 antibody drug targets cells with the non-edited CD45, but cannot bind to HSCs with an edited CD45. Thus, HSCs native to the subject are targeted by anti-CD45 antibody, but gene edited HSCs are not. Thus, both cells express CD45 polypeptides that function in signal transduction. However, binding of the antibody-drug conjugate to native CD45-expressing cells results in death of the cells. In contrast, gene-edited HSCs are not targeted by the anti-CD45 antibody-drug conjugate because the amino acid substitution introduced in CD45 prevents the binding of anti-CD45 antibody, but does not interfere with normal functioning of CD45.

Methods of identifying a candidate agent for selectively depleting or ablating an endogenous stem cell population are also within the scope of the disclosure. Such methods may comprise the steps of: (a) contacting a sample comprising the stem cell population with a test agent (e.g., antibody or antibody-drug conjugate); and (b) detecting whether one or more cells of the stem cell population are depleted or ablated from the sample; wherein the depletion or ablation of one or more cells of the stem cell population following the contacting step identifies the test agent as a candidate agent. In a further step an edited cell is identified as not similarly depleted or ablated by the agent. In some embodiments, the cell is contacted with the test agent for at least about 2-24 hours.

### Editing of Target Genes

To produce the gene edits described herein, cells (e.g., hematopoietic stem cells (HSCs)) are collected from a subject and contacted with one or more guide RNAs and a nucleobase editor polypeptide comprising a nucleic acid programmable DNA binding protein (napDNAbp) and a cytidine deaminase or adenosine deaminase or comprising one or more deaminases. In some embodiments, cells to be edited are contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes.

In some embodiments, the immune cell is modified using prime editing. Methods for editing polynucleotide sequences using prime editing are well known in the art (see, e.g., Petrova I O, Smimikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes). A prime editor construct and paired prime editing guide RNA (pegRNA), with or without a secondary nicking guide RNA (nRNA), can induce targeted, programmable changes to genomic DNA.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

Exemplary guide sequences are provided in Tables 1 and 2.

Variants of the spacer sequences listed in Tables 1 or 2 comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

TABLE 1
Representative guide sequences relating to missense mutations identified by
yeast display screen.

| | | | | Missense |
|---|---|---|---|---|
| | | SEQ | | Mutation to |
| guide_name | Spacer | ID NO | PAM | CD45 |
| GuideDesign4_ABE_NGG_20nt_3- | GCUAAAUGUUAA | 448 | TGG | N253G, N255G |
| 12_018_+_198706800_spCas9 | UGAGAAUG | | | |
| GuideDesign4_ABE NGG_20nt_3- | GUUAAUGAGAAU | 449 | TGG | N255G, E256G, |
| 12_018_+_198706800_spCas9 | GUGGAAUG | | | N257G |
| GuideDesign4_ABE_NGA_20nt_3- | UUAAUGAGAAUG | 450 | GGA | N255G, E256G, |
| 9_005_+_198706808_spCas9 | UGGAAUGU | | | N257D |
| GuideDesign4_ABE_VTTN_22nt_5- | AUGAGAAUGUGG | 451 | GTTA | N257G |
| 9_017_+_198706807_cas12b | AAUGUGGAAA | | | |
| GuideDesign4_ABE_NGC_20nt_3- | UGGAAUGUGGAA | 452 | TGC | E259G |
| 9_008_+_198706820_spCas9 | ACAAUACU | | | |
| GuideDesign4_ABE_NGC_20nt_3- | UGGAAUGUGGAA | 453 | TGC | E259G, N262D |
| 12_020_+_198706820_spCas9 | ACAAUACU | | | |
| GuideDesign4_ABE_NGA_20nt_3- | AACAAUACUUGC | 454 | TGA | N263G, T264A |
| 12_019_+_198706831_spCas9 | ACAAACAA | | | |
| GuideDesign4_ABE_NGG_20nt_3- | CAAUACUUGCAC | 455 | AGG | N263S, T264A |
| 9_002_+_198706833_spCas9 | AAACAAUG | | | |
| GuideDesign4_ABE_VTTN_22nt_5- | CACAAACAAUGA | 456 | CTTG | N267G, N268G |
| 9_017_+_198706838_cas12b | GGUGCAUAAC | | | |
| GuideDesign4_ABE_NGC_20nt_3- | UACUUGCACAAA | 457 | TGC | T266A |
| 9_008_+_198706836_spCas9 | CAAUGAGG | | | |
| GuideDesign4_ABE_NGC_20nt_3- | UACUUGCACAAA | 458 | TGC | T266A, N267G |
| 12_020_+_198706836_spCas9 | CAAUGAGG | | | |
| GuideDesign4_ABE_NGA_20nt_3- | CAUAAUUCAUGU | 459 | TGA | N286G |
| 12_019_+_198706900_spCas9 | ACUGCUCC | | | |
| GuideDesign4_ABE_NGC_20nt_3- | AUAUCUCAUAAU | 460 | TGC | I283M, H285R, |
| 12_020_+_198706894_spCas9 | UCAUGUAC | | | N286G |
| GuideDesign4_ABE_NGC_20nt_3- | AUAUCUCAUAAU | 461 | TGC | I283M, H285R |
| 9_008_+_198706894_spCas9 | UCAUGUAC | | | |
| GuideDesign4_ABE_VTTN_22nt_5- | CCAUAUCUCAUA | 462 | GTTT | I283M |
| 9_017_+_198706888_cas12b | AUUCAUGUAC | | | |

| | | gRNA | gRNA |
|---|---|---|---|
| | | code | code Lead |
| guide_name | editor | Heme | Discovery |
| GuideDesign4_ABE_NGG_20nt_3- | ABE_NGG_20nt_3- | gRNA1612 | gRNA2739 |
| 12_018_+_198706800_spCas9 | 12_018 | | |
| GuideDesign4_ABE_NGG_20nt_3- | ABE_NGG_20nt_3- | gRNA1613 | N/A |
| 12_018_+_198706807_spCas9 | 12_018 | | |
| GuideDesign4_ABE_NGA_20nt_3- | ABE_NGA_20nt_3- | N/A | N/A |
| 9_005_+_198706808_spCas9 | 9_005 | | |
| GuideDesign4_ABE_VTTN_22nt_5- | ABE_VTTN_22nt 5- | N/A | N/A |
| 9_017_+_198706807_cas12b | 9_017 | | |
| GuideDesign4_ABE NGC_20nt_3- | ABE_NGC_20nt_3- | gRNA2696 | gRNA2784 |
| 9_008_+_198706820_spCas9 | 9_008 | | |
| GuideDesign4_ABE NGC 20nt_3- | ABE_NGC_20nt_3- | gRNA2696 | gRNA2784 |
| 12_020_+_198706820_spCas9 | 12_020 | | |
| GuideDesign4_ABE NGA 20nt_3- | ABE_NGA_20nt_3- | N/A | gRNA4366 |
| 12 019_+_198706831_spCas9 | 12_019 | | |
| GuideDesign4_ABE_NGG_20nt_3- | ABE_NGG_20nt_3- | gRNA1614 | gRNA1614 |
| 9_002_+_198706833_spCas9 | 9_002 | | |
| GuideDesign4_ABE_VTTN_22nt_5- | ABE_VTTN_22nt_5- | N/A | N/A |
| 9_017_+_198706838_cas12b | 9_017 | | |
| GuideDesign4_ABE_NGC_20nt_3- | ABE_NGC_20nt_3- | gRNA2863 | gRNA4369 |
| 9_008_+_198706836_spCas9 | 9_008 | | |
| GuideDesign4_ABE_NGC_20nt_3- | ABE_NGC_20nt_3- | gRNA2863 | gRNA4369 |
| 12_020_+_198706836_spCas9 | 12_020 | | |
| GuideDesign4_ABE_NGA_20ntv3- | ABE_NGA_20nt_3- | N/A | N/A |
| 12_019_+_198706900_spCas9 | 12_019 | | |
| GuideDesign4_ABE_NGC_20nt_3- | ABE_NGC_20nt_3- | gRNA2862 | gRNA4393 |
| 12_020_+_198706894_spCas9 | 12_020 | | |
| GuideDesign4_ABE_NGC_20nt_3- | ABE_NGC_20nt_3- | gRNA2862 | gRNA4393 |
| 9_008_+_198706894_spCas9 | 9_008 | | |
| GuideDesign4_ABE_VTTN_22nt_5- | ABE_VTTN_22nt_5- | N/A | N/A |
| 9_017_+_198706888_cas12b | 9_017 | | |

TABLE 2
Representative guide sequences. Guides tested on CD34, Jurkat, Molm13
and/or CD34+ hematopoietic stem and progenitor cell (HSPC) cells.

| | | | | Full Target | | | |
|---|---|---|---|---|---|---|---|
| | | | Predicted | Sequence | | | |
| | | Base | amino acid | (PAM in | SEQ | | SEQ ID |
| gRNA | mRNA | editor¹ | substitutions | bold) | ID NO | Spacer | NO |
| gRNA1610 | 2080 | ABE- | K231R, | AAAATATGC | 463 | AAAAUAUGCAA | 473 |
| | | NGG | Y232C | AAACATCAC | | ACAUCACUG | |
| | | | | TGTGG | | | |
| gRNA1611 | 2080 | ABE- | D238G, | TGTGGATTA | 464 | UGUGGAUUACU | 474 |
| | | NGG | Y239C | CTTATATAA | | UAUAUAACA | |
| | | | | CAAGG | | | |
| gRNA1613 | 2080 | ABE- | N255G, | GTTAATGAG | 465 | GUUAAUGAGAA | 475 |
| | | NGG | E256G | AATGTGGAA | | UGUGGAAUG | |
| | | | | TGTGG | | | |
| gRNA1614 | 2080 | ABE- | N263S, | CAATACTTG | 466 | CAAUACUUGCA | 476 |
| | | NGG | T264A | CACAAACAA | | CAAACAAUG | |
| | | | | TGAGG | | | |
| gRNA2696 | 2743; | ABE- | E259G | TGGAATGTG | 467 | UGGAAUGUGGA | 477 |
| | 3626; | NGC | | GAAACAATA | | AACAAUACU | |
| | 3167 | | | CTTGC | | | |
| gRNA2783 | 2518; | ABE- | E259G | AATGTGGAA | 468 | AAUGUGGAAUG | 478 |
| | 383 | NRCH | | TGTGGAAAC | | UGGAAACAA | |
| | | | | AATACT | | | |
| gRNA4630 | 1570; | ABE- | E259G | TGGAATGTG | 469 | UGGAAUGUGGA | 479 |
| | 2518; | NRCH; | | GAAACAATA | | AACAAUACU | |
| | 383; | ABE- | | CTTGCA | | | |
| | 3626; | NGC | | | | | |
| | 3167 | | | | | | |
| gRNA4373 | 1570; | ABE- | N267G, | GCACAAACA | 470 | GCACAAACAAU | 480 |
| | 2518; | NRCH | T266A, | ATGAGGTGC | | GAGGUGCAU | |
| | 383 | | N268D | ATAACC | | | |
| gRNA4361 | 1570; | ABE- | N257G, | ATGAGAATG | 471 | AUGAGAAUGUG | 481 |
| | 383; | NRCH | E256G | TGGAATGTG | | GAAUGUGGA | |
| | 2518 | | | GAAACA | | | |
| gRNA4359 | 4071; | ABE- | N255G, | TTAATGAGA | 472 | UUAAUGAGAAU | 482 |
| | 2626; | NGA; | E256G, | ATGTGGAAT | | GUGGAAUGU | |
| | 1570 | ABE- | N257D | GTGGA | | | |
| | | NRCH | | | | | |

| | | SEQ ID |
|---|---|---|
| gRNA | gRNA² | NO |
| gRNA2783 | mAsmAsmUsGUGGAAUGUGGAAACAAGUUUUAGAGCUAGAAAUAGCAAGU | 483 |
| | UAAAAUAAGGCUAGUCCGUUAUCAACUUGAAAAAGUGGCACCGAGUCGGU | |
| | GCmUsmUsmUsU | |
| gRNA4630 | mUsmGsmGsAAUGUGGAAACAAUACUGUUUUAGAmGmCmCmGmGmCmGmG | 484 |
| | mAmAmAmCmGmCmCmGmGmCAAGUUAAAAUAAGGCUAGUCCGUUAmUmCA | |
| | AmCmUmUGGACUUCGGUCCmAmAmGUGGmCmAmCmCmGmAmGmUmCmGmG | |
| | mUmGmCmUsmUsmUsmU | |
| gRNA2696 | mUsmGsmGsAAUGUGGAAACAAUACUGUUUUAGAGCUAGAAAUAGCAAGU | 485 |
| | UAAAAUAAGGCUAGUCCGUUAUCAACUUGAAAAAGUGGCACCGAGUCGGU | |
| | GCUsmUsmUsmU | |
| gRNA4373 | mGsmCsmAsCAAACAAUGAGGUGCAUGUUUUAGAGCUAGAAAUAGCAAGU | 486 |
| | UAAAAUAAGGCUAGUCCGUUAUCAACUUGAAAAAGUGGCACCGAGUCGGU | |
| | GCmUsmUsmUsU | |
| gRNA4361 | mAsmUsmGsAGAAUGUGGAAUGUGGAGUUUUAGAGCUAGAAAUAGCAAGU | 487 |
| | UAAAAUAAGGCUAGUCCGUUAUCAACUUGAAAAAGUGGCACCGAGUCGGU | |
| | GCmUsmUsmUsU | |
| gRNA4359 | mUsmUsmAsAUGAGAAUGUGGAAUGUGUUUUAGAGCUAGAAAUAGCAAGU | 488 |
| | UAAAAUAAGGCUAGUCCGUUAUCAACUUGAAAAAGUGGCACCGAGUCGGU | |
| | GCUsmUsmUsmU | |
| gRNA1613 | mGsmUsmUsAAUGAGAAUGUGGAAUGGUUUUAGAGCUAGAAAUAGCAAGU | 489 |
| | UAAAAUAAGGCUAGUCCGUUAUCAACUUGAAAAAGUGGCACCGAGUCGGU | |
| | GCUsmUsmUsmU | |
| GuideDesign4_ | mGsmUsmUsCUGUCUUUUGGUCAGGACAACCGUCUAGCUAUAAGUGCUGC | 490 |
| ABE_VTTN_22nt_5- | AGGGUGUGAGAAACUCCUAUUGCUGGACGAUGUCUCUUACGAGGCAUUAG | |
| 9_017_+_198706838_cas12b | CACCACAAACAAUGAGGUGCAUsmAsmAsmC | |
| GuideDesign4_ | mUsmAsmCsUUGCACAAACAAUGAGGGUUUUAGAGCUAGAAAUAGCAAGU | 491 |
| ABE_NGC_20nt_3- | UAAAAUAAGGCUAGUCCGUUAUCAACUUGAAAAAGUGGCACCGAGUCGGU | |
| 12_020_+_198706836_spCas9 | GCUsmUsmUsmU | |

¹The term following the hyphen “-” indicates the PAM specificity of the base editor. All of the base editors referenced in the table contained a nucleic acid programmable DNA binding domain derived from SpCas9.
²The term “Ns,” where “N” is any nucleotide, indicates that the nucleotide N contains a 3′-phosphorothioate modification such that the nucleotide is linked to the nucleotide 3′ of and adjacent to N by a phorphorothioate group, and the term “mN,” where “N” is any nucleotide, indicates that N contains a 2′-O-methyl modification. In some embodiments the 3′ nucleotide of a gRNA contains a 2′-O-methyl modification. In some embodiments the 3′ nucleotide of a gRNA does not contain a 2′-O-methyl modification.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of abase editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGT, NGCG, NGAG, NGAN, NGNG, NGCN, NGCG, NGTN, NNGRRT, NNNRRT, NNGRR(N), TTTV, TYCV, TYCV, TATV, NNNNGAT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in &T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase or cytidine deaminase) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in SEQ ID NO: 197. Cas9 polypeptide regions comprising a higher than average B-factor can include, for example, residues 792-872, 792-906, and 2-791 as numbered in SEQ ID NO: 197.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in SEQ ID NO: 197, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in SEQ ID NO: 197, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in SEQ ID NO: 197, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or anon-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

(SEQ ID NO: 262)
ATGGCCCCAAAGAAGAAGCGGAAGGTCGGTATCCACGGAGTCCCAGCAG
CC.

In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | D | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of M1I, M1S, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, I49G, I49H, I49V, I49F, I49H, I49I, I49M, I49N, I49K, I49Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, I176I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, I95S, I95G, I95L, I95H, I95V, H96A, H96L, H96R, H96S, H96H, H96N, H96E, S97C, S97G, S97I, S97M, S97R, S97S, S97P, R98K, R98I, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K110I, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L 121M, L121N, L 121K, L 121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M126O, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I132I, I132F, I132T, I132L, I132V, I132E, T133V, T133E, T133G, T133K, T133T, T133A, T133H, T133F, T133I, E134A, E134E, E134G, E134I, E134H, E134K, E134T, G135G, G135V, G135I, G135P, G135E, I136G, I136L, I136T, I136I, I137A, I137D, I137E, L137M, I137S, L137L, L137I, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L 145F, L 145G, L 145D, L 145L, L 145C, L 145E, L 145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D147I, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R152O, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D167I, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “in” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. In an embodiment, the multiple gRNA sequences can be tandemly arranged and are separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some embodiments, cellular DNA repair response to the presence of U: G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a bamase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a sterile alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voβ, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + 176Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)n to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence:

(SEQ ID NO: 361)
SGGSSGGSSGSETPGTSESATPESSGGSSGGSSGGSSGGSSGSETPGTSE
SATPESSGGSSGGS.

In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEEG
TSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.
Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cel

Fusion proteins or complexes of the disclosure comprising an adenosine deaminase variant may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes is reduced markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like can be used.

When the host is Escherichia coli, a trp promoter, lac promoter, recA promoter, .lamda.P.sub.L promoter, lpp promoter, T7 promoter, and the like can be used.

When the host is in the genus Bacillus, the SPO1 promoter, SPO2 promoter, penP promoter, and the like can be used.

When the host is a yeast, the Gall/10 promoter, PHO5 promoter, PGK promoter, GAP promoter, ADH promoter, and the like can be used.

When the host is an insect cell, the polyhedrin promoter, P10 promoter, and the like can be used.

When the host is a plant cell, the CaMV35S promoter, CaMV19S promoter, NOS promoter, and the like can be used.

Expression vectors for use in the present disclosure, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

Host cells of interest, include but are not limited to bacteria, yeast, fungi, insects, plants, and animal cells. For example, a host cell may comprise bacteria from the genus Escherichia, such as Escherichia coli K12.cndot.DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like.

A host cell may comprise bacteria from the genus Bacillus, for example Bacillus subtilis M1114 [Gene, 24, 255 (1983)], Bacillus subtilis 207-21 [Journal of Biochemistry, 95, 87 (1984)] and the like.

A host cell may be a yeast cell. Examples of yeast cells include Saccharomyces cerevisiae AH22, AH22R.sup.-, NA87-11A, DKD-5D, 20B-12, Schizosaccharomyces pombe NCYC1913, NCYC2036, Pichia pastoris KM71 and the like.

When the viral delivery methods utilize the virus AcNPV, cells from a cabbage armyworm larva-derived established line (Spodoptera frugiperda cell; Sf cell), MG1 cells derived from the mid-intestine of Trichoplusia ni, High Five™ cells derived from an ovary of Trichoplusia ni, Mamestra brassicae-derived cells, Estigmene acrea-derived cells and the like can be used. When the virus is BmNPV, cells of Bombyx mori-derived established line (Bombyx mori N cell; BmN cell) and the like are used. As the Sf cell, for example, Sf9 cell (ATCC CRL1711), Sf21 cell [all above, In Vivo, 13, 213-217 (1977)] and the like are used.

An insect can be any insect, for example, larva of Bombyx mori, Drosophila, cricket, and the like [Nature, 315, 592 (1985)].

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Plant cells are also contemplated in the present disclosure. Plant cells include, but are not limited to, suspended cultured cells, callus, protoplast, leaf segment, root segment and the like prepared from various plants (e.g., grain such as rice, wheat, com, and the like; product crops such as tomato, cucumber, eggplant and the like; garden plants such as carnations, Eustoma russellianum, and the like; and other plants such as tobacco, Arabidopsis thaliana and the like) are used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂ coprecipitation method, electroporation, microinjection, particle gun method, lipofection, Agrobacterium-mediated delivery, etc.) according to the kind of the host.

Escherichia coli can be transformed according to the methods described in, for example, Proc. Natl. Acad. Sci. USA, 69, 2110 (1972), Gene, 17, 107 (1982).

The genus Bacillus can be introduced into a vector according to the methods described in, for example, Molecular & General Genetics, 168, 111 (1979).

A yeast can be introduced into a vector according to the methods described in, for example, Methods in Enzymology, 194, 182-187 (1991), Proc. Natl. Acad. Sci. USA, 75, 1929 (1978).

An insect cell and an insect can be introduced into a vector according to the methods described in, for example, Bio/Technology, 6, 47-55 (1988).

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host. For example, when Escherichia coli or genus Bacillus is cultured, a liquid medium may be used for the culture. In an embodiment, the medium contains a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is between about 5 and about 8.

As a medium for culturing Escherichia coli, for example, M9 medium containing glucose, casamino acid [Journal of Experiments in Molecular Genetics, 431-433, Cold Spring Harbor Laboratory, New York 1972] is one that may be used. Where necessary, for example, agents such as 3β-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30 to about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium may be from about 5 to about 8. The culture is performed at generally about 20° C. to about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. In an embodiment, the pH of the medium may be from about 6.2 to about 6.4. The culture is performed at generally about 27° C. Where necessary, aeration and stirring may be performed.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium is about 6 to about 8. The culture is performed at generally about 30° C. to about 40° C. Where necessary, aeration and stirring may be performed.

As a medium for culturing a plant cell, for example, MS medium, LS medium, B5 medium and the like are used. The pH of the medium is about 5-about 8. The culture is performed at generally about 20° C. to about 30° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Prokaryotic cells such as Escherichia coli and the like can utilize an inducible promoter. Examples of the inducible promoter include, but are not limited to, lac promoter (induced by IPTG), cspA promoter (induced by cold shock), araBAD promoter (induced by arabinose) and the like.

Alternatively, the above-mentioned inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Immunoconjugates

In some embodiments, an anti-CD45 antibody of the disclosure is or is part of an immunoconjugate (“anti-CD45 antibody immunoconjugate”), in which the anti-CD45 antibody is conjugated to one or more heterologous molecule(s), such as, but not limited to, a cytotoxic or an imaging agent. The fusion of the cytotoxic agent with the anti-CD45 antibody may have therapeutic value. Cytotoxic agents include, but are not limited to, radioactive isotopes (e.g., At²¹¹, I¹³¹, I¹²⁵, Y⁹⁰, Rel⁸⁶, Rel⁸⁸, Sml⁵³, Bi²¹², P³², Pb²¹² and radioactive isotopes of Lu); chemotherapeutic agents (e.g., maytansinoids, taxanes, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Among the anti-CD45 antibody immunoconjugates are antibody-drug conjugates (ADCs), in which an anti-CD45 antibody is conjugated to one or more drugs, including but not limited to a maytansinoid (see U.S. Pat. Nos. 5,208,020, 5,416,064 and European Patent EP 0 425 235 B 1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53: 3336-3342 (1993); and Lode et al, Cancer Res. 58: 2925-2928 (1998)); an anthracycline such as daunomycin or doxorubicin (see Kratz et al., Current Med. Chem. 13: 477-523 (2006); Jeffrey et al., Bioorganic & Med. Chem. Letters 16: 358-362 (2006); Torgov et al., Bioconj. Chem. 16: 717-721 (2005); Nagy et al, Proc. Natl. Acad. Sci. USA 97: 829-834 (2000); Dubowchik et al, Bioorg. & Med. Chem. Letters 12: 1529-1532 (2002); King et al, J. Med. Chem. 45: 4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

Also among the anti-CD45 antibody immunoconjugates are those in which the antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the anti-CD45 antibody is conjugated to a protein degrader, such as those described in Frere, G., et al. Methods in Cell Biology, 167:1-26 (2022) and Sasso, J., et al. Biochemistry, 62:601-623 (2023), or in International Patent Applications No. WO 2021/053555, WO 2021/249517, WO 2020/006264, WO 2008/115516, WO 2021/126805, WO 2021/178920, WO 2021/127080, WO 2014/094138, WO 2015/200795, WO 2017/117118, and WO 2020/079103 the disclosures of which is incorporated herein by reference in their entireties for all purposes. In some embodiments, the degrader is CC-122, CC-220, CC-99282, CFT7455, DKY709, CR8, Glue01, HQ005, FPFT-2216, TMX-4116, Eragidomide, BTX-1188, MG-277, ZHX-1-161, Indisulam, E7820, dCeMM1, CQS, NRX-252114, NRX-252262, BI-3802, CCT369260, Cyclosporin A, Lupkynis, Sanglifehrin A, Auxin, Jasmonate, lenalidomide (Revlimid), lenalidomide, pomalidomide (Pomalyst), or thalidomide. Non-limiting examples of types of protein degraders suitable for use in compositions, conjugates, and/or methods of the disclosure include heterobifunctional degraders and molecular glue degraders.

Also among the anti-CD45 antibody immunoconjugates are those in which the anti-CD45 antibody is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

Conjugates of an anti-CD45 antibody and cytotoxic agent may be made using any of a number of known protein coupling agents, e.g., linkers, (see Vitetta et al., Science 238: 1098 (1987)). The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52: 127-131 (1992); U.S. Pat. No. 5,208,020).

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application Publication No. US 2022/0290164 A1, the disclosure of which is incorporated herein by reference in its entirety for all purposes), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6, AAV9, PHP.EB, PHP.B, AAV.CAP-B10, AAV, CAP-B22, AAV-rh10, a PAL family AAV, or any combination thereof. In embodiments, the AAV is capable of crossing the blood-brain barrier (see, e.g., those AAV vectors disclosed in Liu, et al. “Crossing the blood-brain barrier with AAV vectors,” Metabolic Brain Disease, 36:45-52 (2021), the disclosure of which is incorporated herein by reference in its entirety for all purposes). One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)). In some embodiments, the AAV vector contains a PAL family AAV capsid (see, Stanton, A., et al. Med 4:31-50 (2023) (doi: doi.org/10.1016/j.medj.2022.11.002), the disclosure of which is incorporated herein by reference in its entirety for all purposes).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (lssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publications No. WO 2013/045632, WO 2024/073385, and WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N-[N-terminal portion of the split Cas9]-[intein-N]-C. In some embodiments, an intein-C is fused to the N-terminus of the C-terminal portion of the split Cas9, i.e., to form a structure of N-[intein-C]-[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminal fragment is fused at the C-terminus to an intein-N and the C-terminal fragment is fused to an intein-C at an N-terminal amino acid selected from the group consisting of S303, T310, T313, S355, A456, S460, A463, T466, S469, T472, T474, C574, S577, A589, and S590, referenced to SEQ ID NO: 197. In various embodiments, the SpCas9 is split between amino acid positions 302 and 303, 309 and 310, 312 and 313, 354 and 355, 455 and 456, 459 and 460, 462 and 463, 465 and 466, 468 and 469, 471 and 472, 473 and 474, 573 and 574, 576 and 577, 588 and 589, or 589 and 590, referenced to SEQ ID NO: 197 to yield an N-terminal fragment and a C-terminal fragment, where the N-terminal fragment is fused at the C-terminus to a an intein-N and where the C-terminal fragment is fused at the N-terminus to an intein-C.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of a disease in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Non-Genotoxic Conditioning

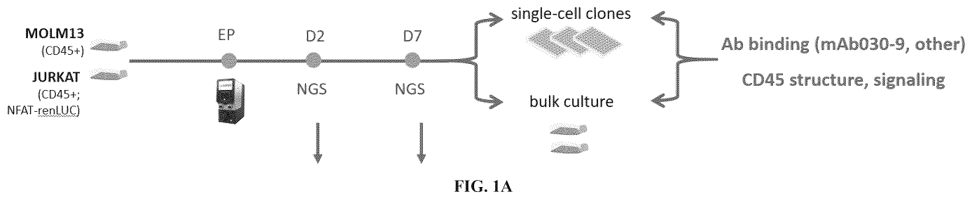

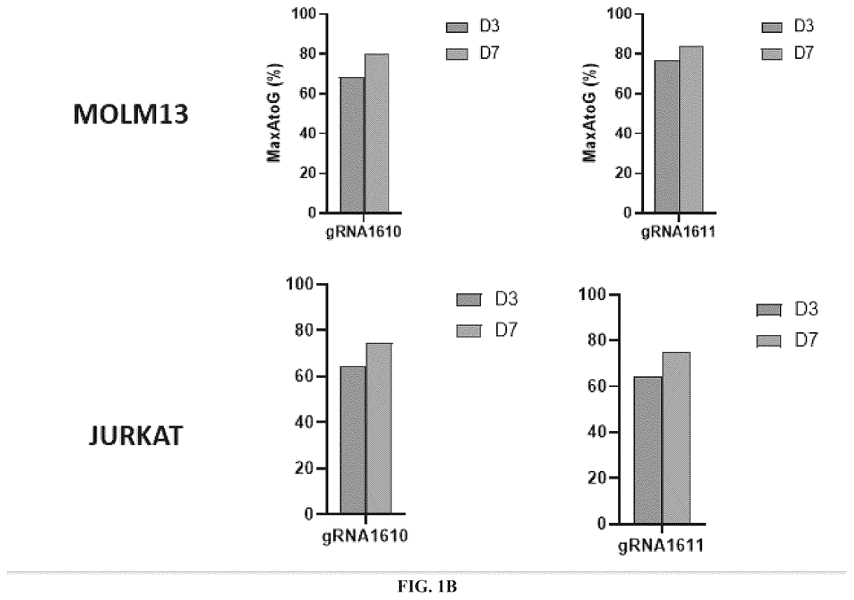

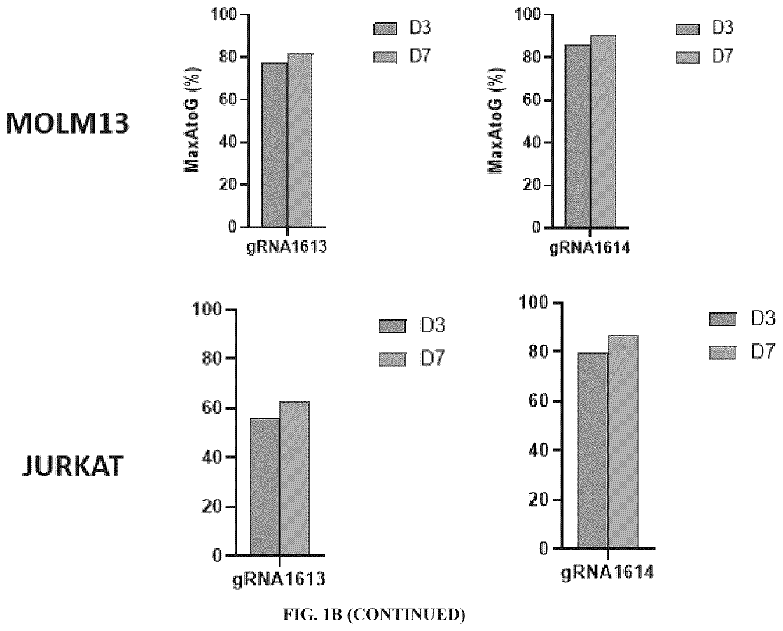

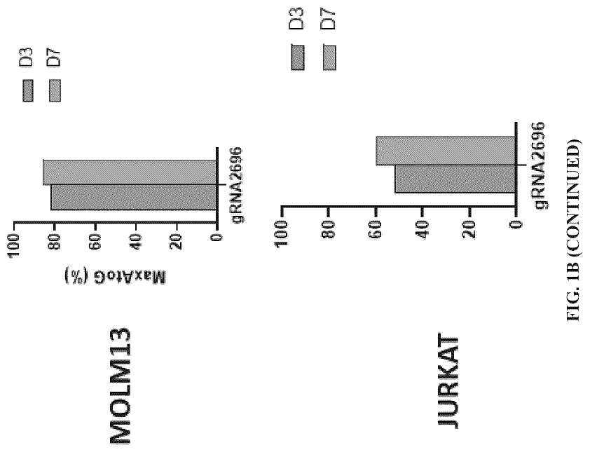

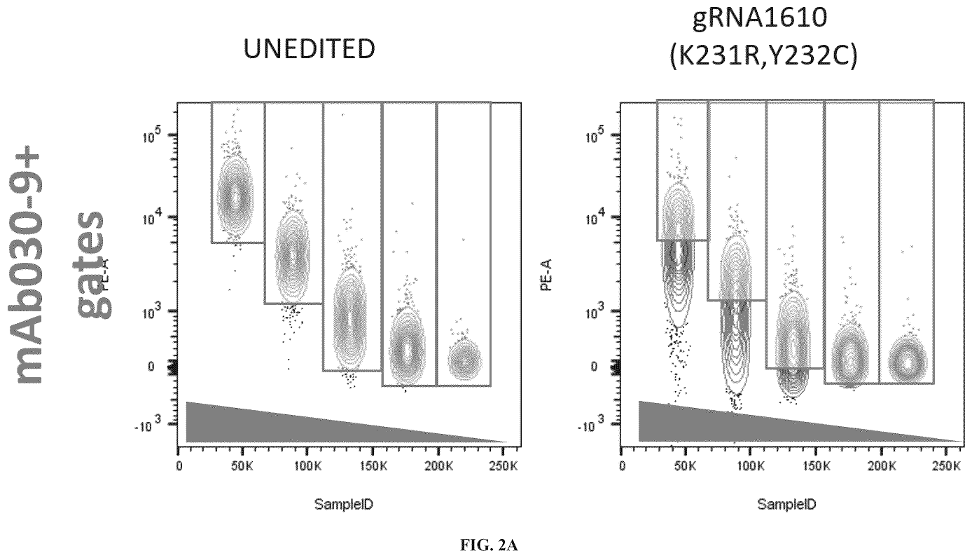

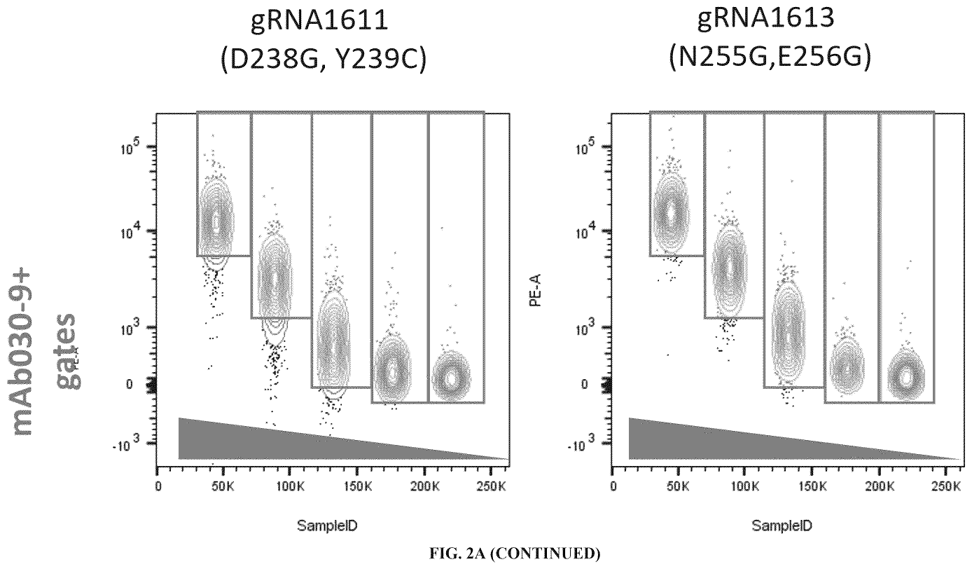

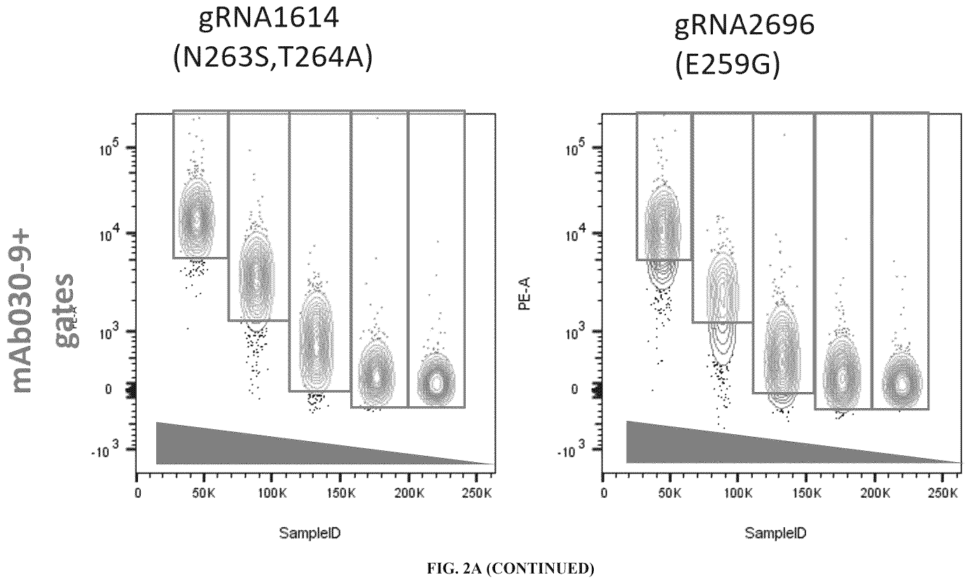

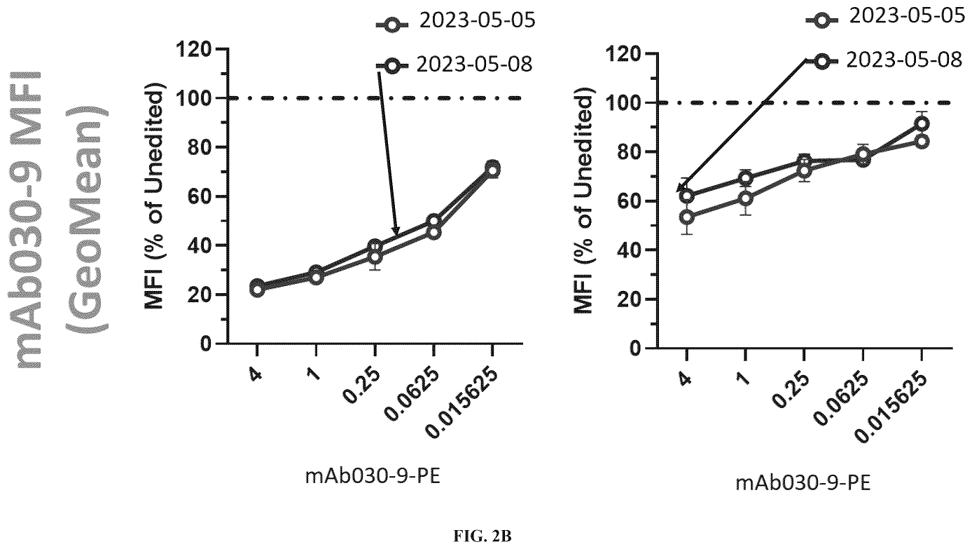

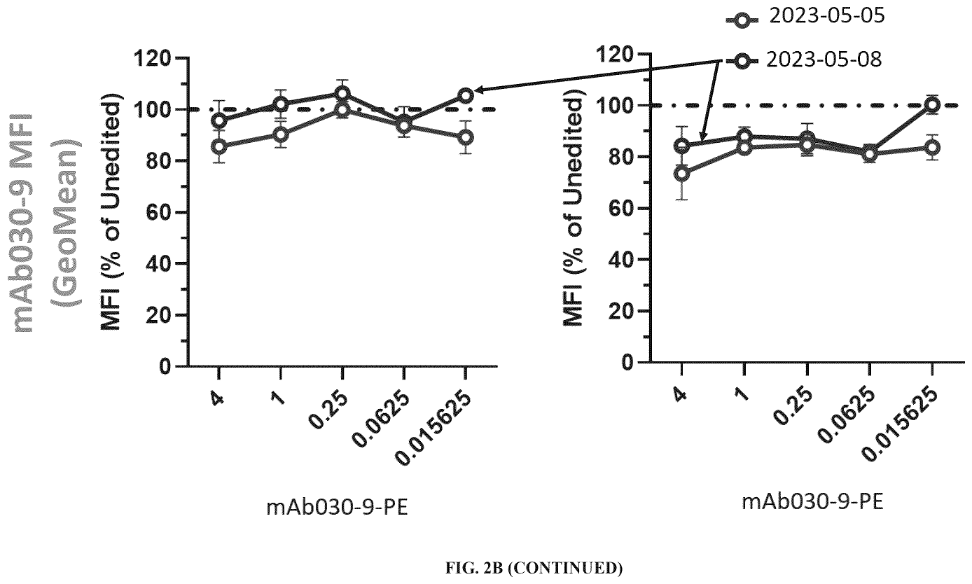

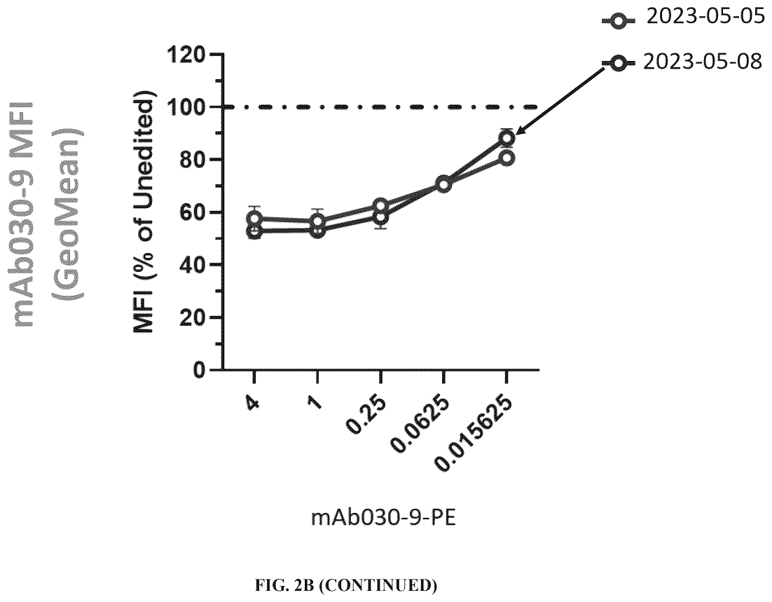

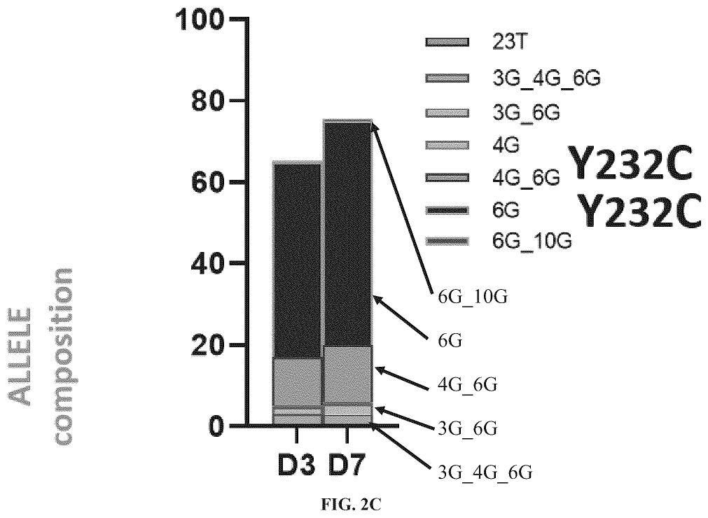

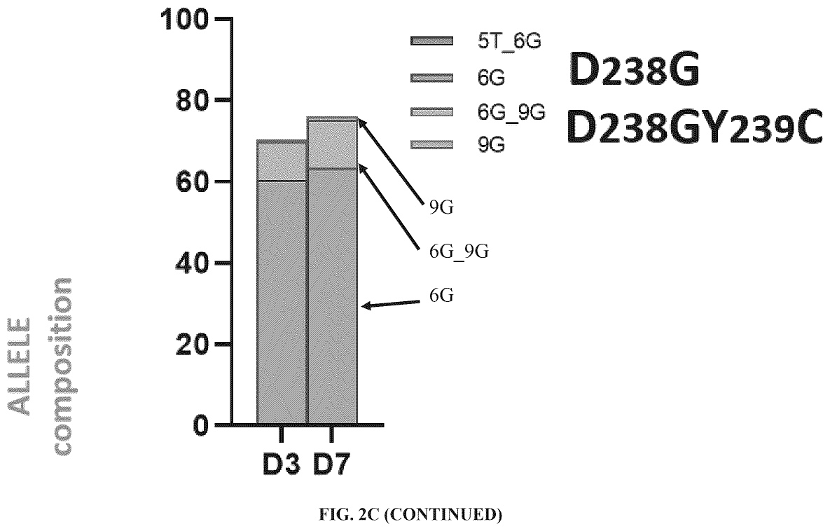

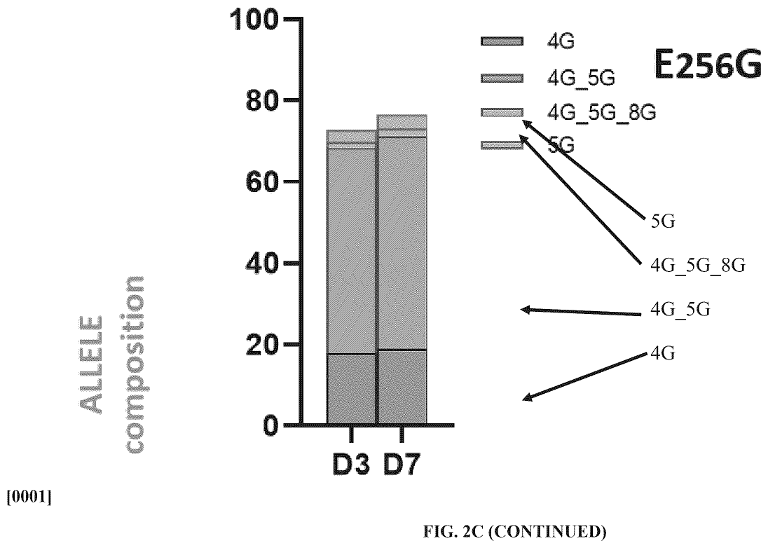

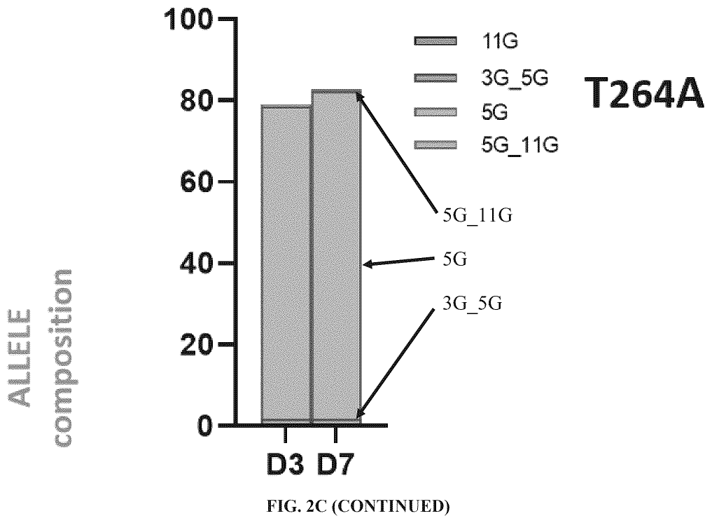

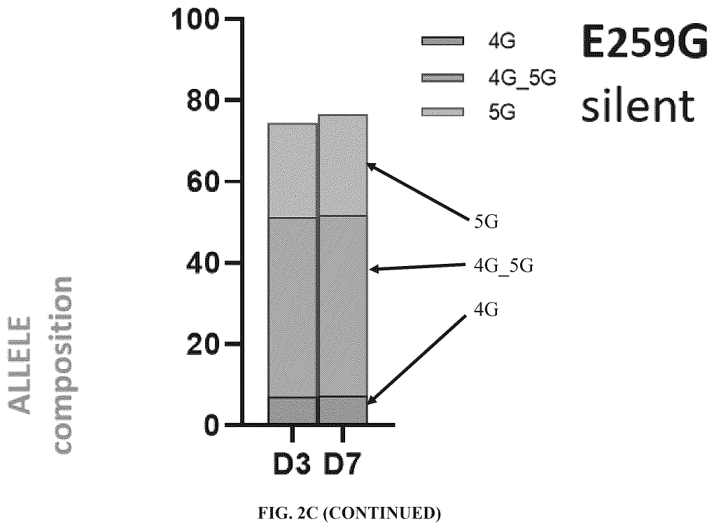

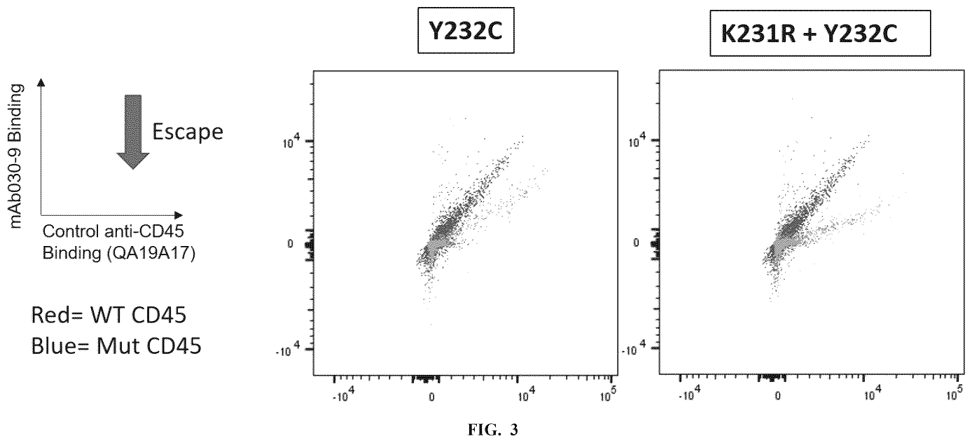

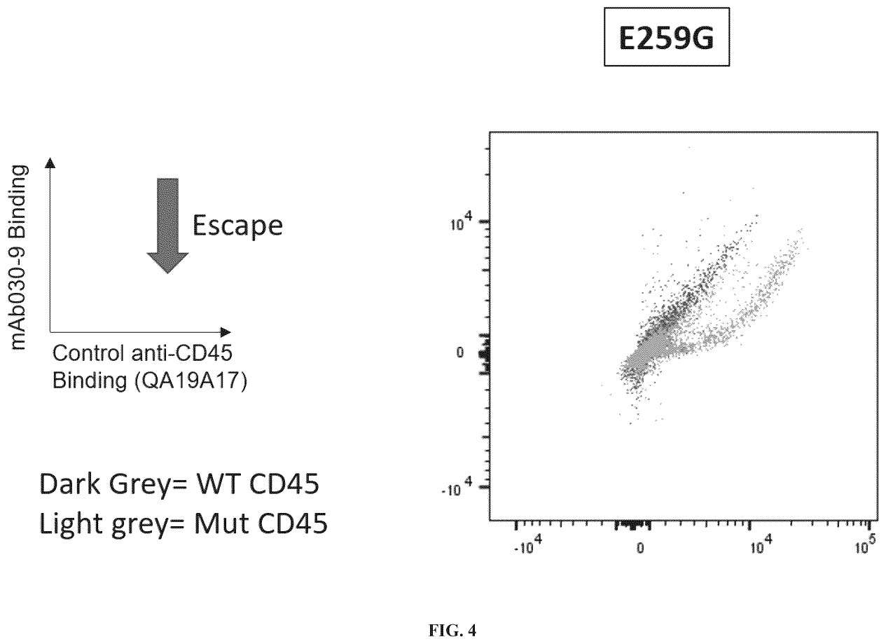

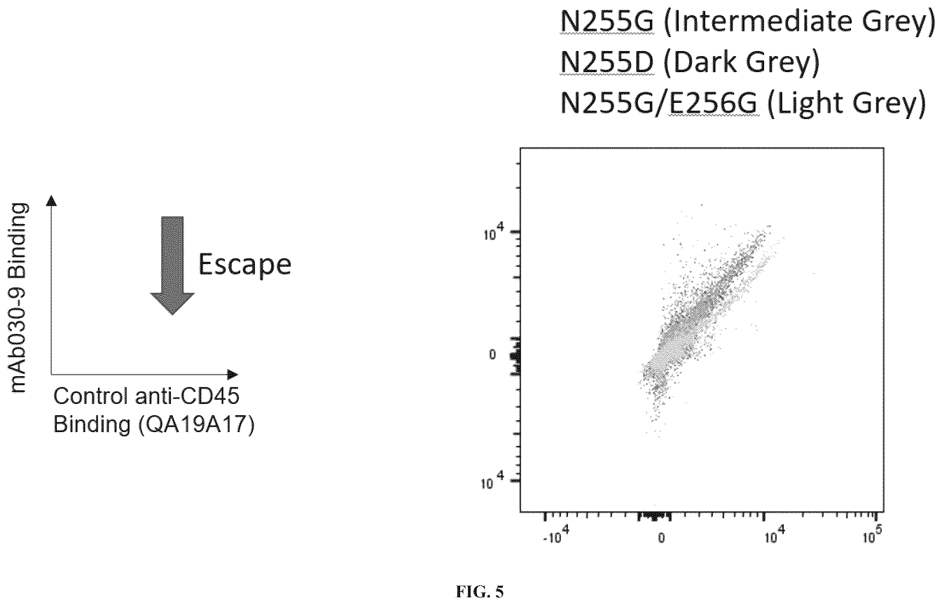

Experiments were undertaken to demonstrate methods for non-genotoxic conditioning. A yeast display screen was used to identify amino acid alterations to a cluster of differentiation 45 (CD45) polypeptide that led to reduced binding by mAb039-9 to the polypeptide (see Table 1). To demonstrate the impact of the amino acid alterations on the binding of mAb030-9 to the CD45 polypeptide, MOLM13 and JURKAT cells were base edited as indicated in FIG. 1A using base editor systems containing mRNA encoding an adenosine deaminase and a base editor selected from gRNA1610, gRNA1611, gRNA1613, gRNA1614, and gRNA2696 (see Table 2). FIG. 13 shows maximum percents of A to G base editing observed in the cells at 3 and 7 days post-electroporation. The base editor systems were associated with maximum percents A to G base editing of as high as about 80% at days 3 and 7, where an increase in base editing rates was observed between days 3 and 7. The edited MOLM 13 cells showed reduced binding to the mAb30-9 antibody (Table 8; FIGS. 2A and 2). FIG. 2C provides stacked bar graphs showing the allele compositions of the edited cells. CD45 variants containing the following alterations showed reduced binding by mAB030-9 (FIGS. 3-5): Y232C; K231R and Y232C; E259G; N255G; N255D; N255G and N256G. Cells base edited to introduce the Y232C, K231R+Y232C, or E259G amino acid substitutions to CD45 showed high frequencies of edits in CD34+ cells. These data demonstrated that base editing can be used to alter a CD45 polynucleotide in a cell to encode a CD45 polypeptide with reduced binding to an anti-CD45 antibody (e.g., mAb030-9).

TABLE 8
Binding of mAbU139AGL030-9 to mutant CD45 D1-D2
protein compared to that of WT CD45 D1-D2.

| CD45 Residue Number | % Mutant Binding |
|---|---|
| N253G, N255G | 44.9 |
| N257G, E256G, N257D | 2 |
| N257G, E256G, N257G | 1.9 |
| N257G, E256G | 8.5 |
| N257G | 1.7 |
| E259G | 2.7 |
| E259G, N262D | 2.3 |
| N263G, T264A | 106.5 |
| N263S, T264A | 121.5 |
| N263S, T264A, T266A | 69.3 |
| T264A | 111.9 |
| T266A | 73.4 |
| T266A, N267G | 1.4 |
| N267G, N268G | 1.3 |
| N286G | 81.5 |
| I283M | 12.9 |
| I283M, H285R | 1 |
| I283M, H285R, N286G | 0.7 |

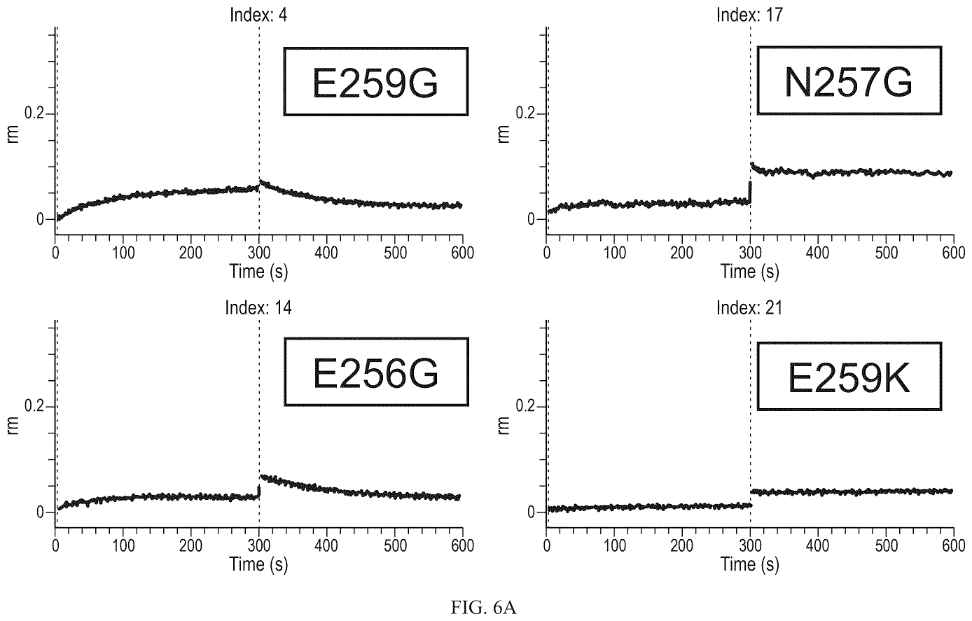

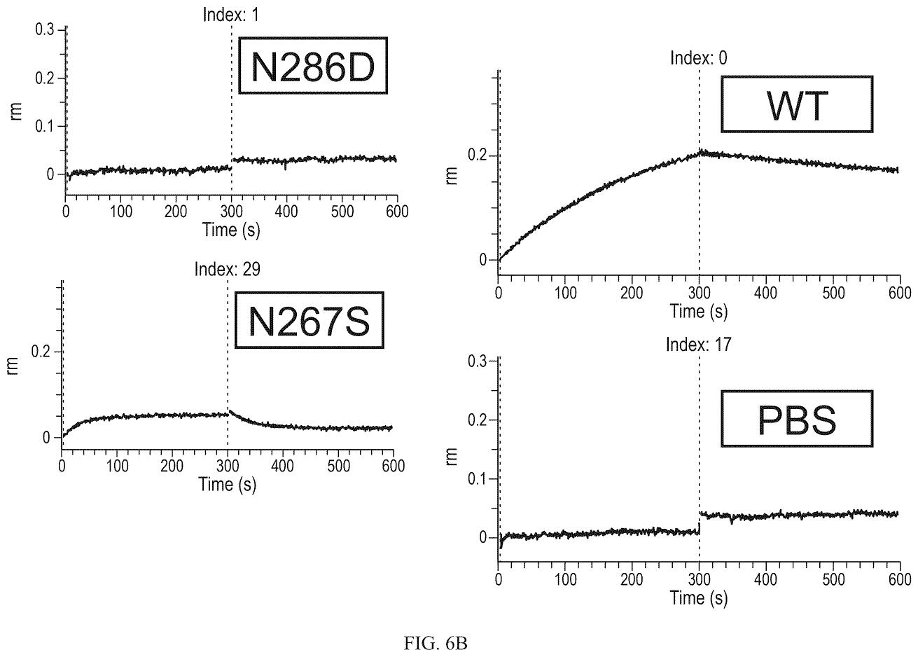

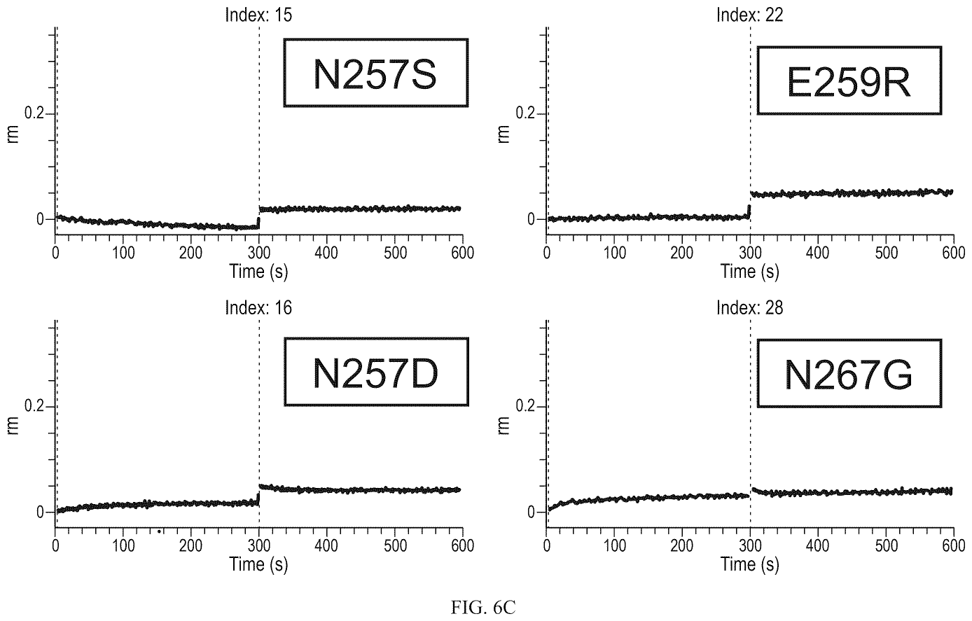

To allow for further evaluation of the CD45 variants showing reduced binding to mAb030-9, the CD45 polypeptides listed in Table 9 and FIG. 6A to 6C were overexpressed and purified. Kinetics of binding of purified CD45 variants containing an alteration selected from E259G, N257G, E256G, E259K, N286D), N267S, N257S, E259R, N2571D, and N267G to mAb030-9 was measured using a biosensor (FIGS. 6A to 6C). The alterations E259R, E259K, and N257S all showed similar or lower response to phosphate buffered saline, and all mutants containing one of the alterations selected from E259G, N257G, E256G, E259K, N286D, N267S, N257S, E259R, N2571D, and N267G had responses lower than 0.05 nm.

TABLE 9
Representative CD45 polypeptides generated using
CD45R0 overexpression constructs in CHO-S cells
Polypeptide Name

CD45R0 tm
CD45R0 tm K231R, Y232C
CD45R0 tmY232C
CD45R0 tm D238G
CD45R0 tm D238G, Y239C
CD45R0 tm N255G
CD45R0 tm N255G,E256G
CD45R0 tm N255D
CD45R0 tm E259G
CD45R0 tm N264S, T265A
CD45R0 tm T265A
CD45R0 tm T265A, T267A

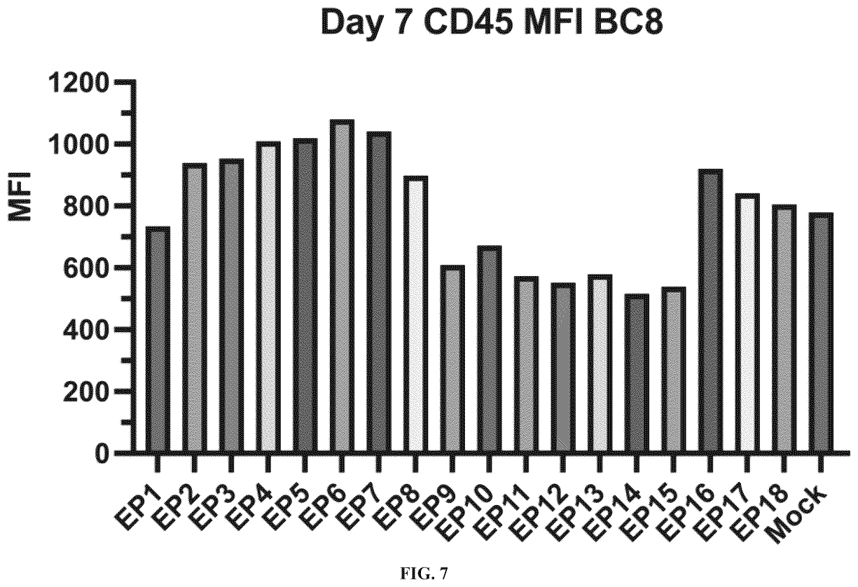

Experiments were undertaken to demonstrate reduced binding by mAb030-9 to cells base edited to express altered CD45 polypeptides. CD34+ HSPC cells were base edited using the base editor systems described in Table 15, and the base editing rates and amino acid alterations associated with each base editor system are listed in Tables 10 to 14, and 16. Cells edited using the base editor systems EP9 to EP15 all showed reduced binding compared to unedited (“Mock”) cells (FIG. 7).

TABLE 10
Editing rates measured for base editor systems evaluated. Nucleotide alterations
are referenced to the spacer sequence for the indicated gRNA.

| | | Base Editing Rates for the Indicated | | | | | | |
|---|---|---|---|---|---|---|---|---|
| | | Nucleotide and Amino Acid Alterations | | | | | | |
| Base | | | | 3G_4G_7G | | 4G_7G | | |
| Editor | | 3G | 3G_4G | N255G | 4G | N255S | 7G | 9G |
| System | gRNA | N255D | N255G | E256G | N255S | E256G | E256G | N257D |
| EP2 | gRNA4359 | 1.2 | 2.42 | 2.53 | 4.67 | 3.69 | 5.98 | 0.2 |
| EP3 | gRNA4359 | 0.01 | 0 | 0 | 0 | 0.01 | 0.13 | 0.27 |

TABLE 11
Editing rates measured for base editor systems
evaluated. Nucleotide alterations are referenced
to the spacer sequence for the indicated gRNA.

| | | Base Editing Rates for the Indicated | | | |
|---|---|---|---|---|---|
| | | Nucleotide and Amino Acid Alterations | | | |
| Base | | | 4G_6G_7G | | |
| Editor | | 13G | E256G | 6G | 6G_7G |
| System | gRNA | E259G | N257G | N257D | N257G |
| EP4 | gRNA4361 | 0.01 | 0.04 | 0.33 | 0.06 |
| EP5 | gRNA4361 | 0.01 | 0.57 | 1.87 | 1.01 |
| EP6 | gRNA4361 | 1.08 | 0 | 0.01 | 0 |

TABLE 12
Editing rates measured for base editor systems
evaluated. Nucleotide alterations are referenced
to the spacer sequence for the indicated gRNA.

| | | Base Editing Rates for the Indicated | | |
|---|---|---|---|---|
| Base | | Nucleotide and Amino Acid Alterations | | |
| Editor | | 8G | 8G_9G | |
| System | gRNA | E259G | E259G | 9G |
| EP7 | gRNA2783 | 1.16 | 0.06 | 0.52 |
| EP8 | gRNA2783 | 12.68 | 1.15 | 1.85 |
| EP9 | gRNA4630 | 17 | 24.46 | 34.09 |

TABLE 13
Editing rates measured for base editor systems evaluated. Nucleotide alterations
are referenced to the spacer sequence for the indicated gRNA.

| | | Base Editing Rates for the Indicated | | | | |
|---|---|---|---|---|---|---|
| Base | | Nucleotide and Amino Acid Alterations | | | | |
| Editor | | 4G | 4G_5G | 4G_5G_11G | 5G | 5G_11G |
| System | gRNA | E259G | E259G | E259G | E259E | E259E |
| EP10 | gRNA4630 | 9.81 | 27.07 | 0.1 | 38.34 | 0.13 |
| EP11 | gRNA4630 | 5.56 | 46.33 | 2.21 | 28.46 | 1.47 |
| EP12 | gRNA4630 | 1.79 | 54.27 | 0.05 | 30.48 | 0.05 |
| EP13 | gRNA4630 | 1.06 | 42.26 | 3.49 | 21.88 | 2.54 |
| EP14 | gRNA2696 | 1.46 | 55.61 | 0.03 | 26.34 | 0.07 |
| EP15 | gRNA2696 | 1.02 | 44.33 | 4.28 | 21.53 | 3.65 |

TABLE 14
Editing rates measured for base editor systems evaluated. Nucleotide alterations are referenced to the spacer sequence for the indicated gRNA.

| | | Base Editing Rates for the Indicated Nucleotide and Amino Acid Alterations | | | | | | | | | | |
|---|---|---|---|---|---|---|---|---|---|---|---|---|
| | | | | | 3G_5G_{—} | | | | | | | |
| | | | 3G_5G_{—} | 3G_5G_{—} | 6G_7G_9G | 3G_5G_{—} | 3G_5G_{—} | | | | | |
| Base | | | 6G | 6G_7G | T266A | 7G | 9G | | | 5G_6G_{—} | | |
| Editor | | 3G_5G | T266A | T266A | N267C | T266A | T266A | | 5G_6G | 7G | 5G_7G | 5G_9G |
| System | gRNA | T266A | N267D | N267G | N268D | N267S | N268D | 5G | N267D | N267G | N267S | N268D |
| EP17 | gRNA4373 | 1.02 | 0.09 | 0.02 | 0 | 0.19 | 0.02 | 9.93 | 0.46 | 0.1 | 0.63 | 0.09 |
| EP18 | gRNA4373 | 2.08 | 1.19 | 2.2 | 2.06 | 0.66 | 0.55 | 7.94 | 1.29 | 0.97 | 0.98 | 0.93 |
| Mock | gRNA4373 | 0 | 0 | 0 | 0 | 0 | 0 | 0.01 | 0.01 | 0 | 0 | 0 |
| | | 3G_5G | | | | | | 5G | | | | |
| EP16 | gRNA4373 | 0.68 | | | | | | 6.5 | | | | |

TABLE 15
Description of base editor systems E1 to E18 and volumes of the
indicated stocks used to transfect cells using electroporation.

| | mRNA | gRNA | | | mRNA stock | sgRNA stock |
|---|---|---|---|---|---|---|
| Base | stock | stock | | | added for | added for |
| Editor | conc. | conc. | | | electroporation | electroporation |
| System | (nM) | (μM) | gRNA | Editor³ | (μl) | (μl) |
| EP1 | 50 | 10 | 4359 | ABE_NGA (4071) | 2 | 2.3 |
| EP2 | 50 | 10 | 4359 | ABE_NGA (2626) | 2 | 2.3 |
| EP3 | 50 | 10 | 4359 | NRCH 8.20 (1570) | 1 | 1.7 |
| EP4 | 50 | 10 | 4361 | NRCH 9 (2518) | 1 | 1.7 |
| EP5 | 50 | 10 | 4361 | NRCH 8e (383) | 0.5 | 1.7 |
| EP6 | 50 | 10 | 4361 | NRCH 8.20 (1570) | 1 | 2.7 |
| EP7 | 50 | 10 | 2783 | NRCH 9 (2518) | 1 | 2.7 |
| EP8 | 50 | 10 | 2783 | NRCH 8e (383) | 0.5 | 2.7 |
| EP9 | 50 | 10 | 4630 | NRCH 8.20 (1570) | 1 | 2.3 |
| EP10 | 50 | 10 | 4630 | NRCH 9 (2518) | 1 | 2.3 |
| EP11 | 50 | 10 | 4630 | NRCH 8e (383) | 0.5 | 2.3 |
| EP12 | 50 | 10 | 4630 | NGC 8.20 (3626) | 2 | 2.3 |
| EP13 | 50 | 10 | 4630 | NGC 8.20 IBE16 | 2 | 2.3 |
| | | | | (3167) | | |
| EP14 | 50 | 10 | 2696 | NGC 8.20 (3626) | 2 | 2.5 |
| EP15 | 50 | 10 | 2969 | NGC 8.20 IBE16 | 2 | 2.5 |
| | | | | (3167) | | |
| EP16 | 50 | 10 | 4373 | NRCH 8.20 (1570) | 1 | 1.8 |
| EP17 | 50 | 10 | 4373 | NRCH 9 (2518) | 1 | 1.8 |
| EP18 | 50 | 10 | 4373 | NRCH 8e (383) | 0.5 | 1.8 |

³The terms “NRCH” and “NGC” indicate the PAM specificity of the napDNAbp domain of the base editor of the base editor system. The term “8.20” indicates a base editor containing a TadA*8.20 adenosine deaminase domain, the term “8e” indicates a base editor containing a TadA-8e adenosine deaminase domain, and the term “ABE” indicates an adenosine base editor containing an adenosine deaminase domain. The numbers in parenthesis identify the mRNA encoding the base editor (see, e.g., Table 17). The term “9” indicates a base editor containing a TadA*8.20 adenosine deaminase domain with the following amino acid substitutions: S82T, Y147D, F149Y, T166I, and D167N. The term “IBE16” indicates that the base editor contained an adenosine deaminase domain inserted within the napDNAbp domain. All of the base editors corresponding to the base editor systems listed in the table contained an SpCas9 nickase domain having the indicated PAM binding specificity.

TABLE 16
Base editing efficiencies for the E259G alteration carried
out using the base editor systems EP9 to EP15.

| Base Editor | Editing Efficiency for the |
|---|---|
| System | E259G Amino Acid Alteration |
| EP9 | 41.46 |
| EP10 | 36.98 |
| EP11 | 54.1 |
| EP12 | 56.11 |
| EP13 | 46.81 |
| EP14 | 57.1 |
| EP15 | 49.63 |

The following materials and methods were employed in the above Example.

### CD34+ Cell Preparation

Mobilized peripheral blood was obtained and enriched for Human CD34+ hematopoietic stem and progenitor cell (HSPC) cells (HemaCare, M001F-GCSF/MOZ-2). The CD34+ HSPC cells were thawed and put into X-VIVO 10 (Lonza) containing 1% Glutamax (Gibco), 100 ng/mL of TPO (Peprotech), SCF (Peprotech) and Flt-3 (Peprotech) at 48 hours prior to electroporation.

### Electroporation of CD34+ Cells

48 hours post thaw, the cells were spun down to remove the X-VIVO 10 media and washed in MaxCyte buffer (HyClone) with 0.1% HSA (Akron Biotechnologies). The cells were then resuspended in cold MaxCyte buffer at 1,250,000 cells per mL and split into multiple 20 μL aliquots. ABE mRNA and guide polynucleotides were then aliquoted as per the experimental conditions (see Table 15) and raised to a total of 5 μL in MaxCyte buffer. The 20 μL of cells was the added into the 5 μL RNA mixture in groups of 3 and loaded into each chamber of an OC25×3 MaxCyte cuvette for electroporation. After receiving the charge, 25 μL was collected from the chambers and placed in the center of the wells in a 24-well untreated culture plate. The cells recovered for 20 minutes in an incubator (37° C., 5% CO₂). After the 20 minutes recovery, X-VIVO 10 (a hematopoietic cell medium) containing 1% Glutamax, 100 ng/mL of TPO, SCF and Flt-3 was added to the cells for a concentration of 1,000,000 cells per mL. The cells were then left to further recover in an incubator (37° C., 5% CO₂) for 48 hrs.

### Genomic DNA Extraction for CD34+ Cells

Following electroporation (48h later), an aliquot of cells was cultured in X-VIVO 10 media (Lonza) containing 1% Glutamax (Gibco), 100 ng/mL of TPO (Peprotech), stem cell factor (SCF) (Peprotech) and Flt-3 (Peprotech). Following 48 h and 144 h post culturing, 100,000 cells were collected and spun down. 50 μL of Quick Extract (Lucigen) was added to the cell pellet and the cell mixture was transferred to a 96-well PCR plate (Bio-Rad). The lysate was heated for 15 minutes at 65° C. followed by 10 minutes at 98° C. The cell lysates were stored at −20° C.

### Kinetics of Binding

100 nM of mAb030-9 immobilized with Anti-Human IgG Fc Capture (AHC) to biosensor. 300 nM of purified CD45 protein was contacted with the immobilized antibody. At 300 s the biosensor was placed in buffer without purified CD45 protein.

### Sequences

Table 17 below provides amino acid sequences for base editors encoded by the indicated mRNA sequences.

TABLE 17
Base editor amino acid sequences encoded by the indicated mRNA sequences.

| | | SEQ |
|---|---|---|
| mRNA | Encoded Amino Acid Sequence | ID NO |
| 1570 | MSEVEFSHEYWMRHALTLAKRARDEREVPVGAVLVLNNRVIGEGWNRAIGLHDPT | 492 |
| | AHAEIMALRQGGLVMQNYRLYDATLYSTFEPCVMCAGAMIHSRIGRVVFGVRNAK | |
| | TGAAGSLMDVLHHPGMNHRVEITEGILADECAALLCRFYRMPRRVFNAQKKAQSS | |
| | TDSGGSSGGSSGSETPGTSESATPESSGGSSGGSDKKYSIGLAIGTNSVGWAVIT | |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMVKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GIIPHQIHLGELHAILRRQGDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRLRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGGHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKGNSDKLIAR | |
| | KKDWDPKKYGGFNSPTVAYSVLVVAKVEKGKSKKLKSVKELLGITIMERSSFEKN | |
| | PIDFLEAKGYKEVKKDLIIKLPKYSLFELENGRKRMLASAGVLQKGNELALPSKY | |
| | VNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTINRKQYNTTKEV | |
| | LDATLIRQSITGLYETRIDLSQLGGDEGADKRTADGSEFESPKKKRKV | |
| 2518 | MSEVEFSHEYWMRHALTLAKRARDEREVPVGAVLVLNNRVIGEGWNRAIGLHDPT | 493 |
| | AHAEIMALRQGGLVMQNYRLYDATLYTTFEPCVMCAGAMIHSRIGRVVFGVRNAK | |
| | TGAAGSLMDVLHHPGMNHRVEITEGILADECAALLCDFYRMPRRVFNAQKKAQSS | |
| | INSGGSSGGSSGSETPGTSESATPESSGGSSGGSDKKYSIGLAIGTNSVGWAVIT | |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMVKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GIIPHQIHLGELHAILRRQGDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRLRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGGHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKGNSDKLIAR | |
| | KKDWDPKKYGGFNSPTVAYSVLVVAKVEKGKSKKLKSVKELLGITIMERSSFEKN | |
| | PIDFLEAKGYKEVKKDLIIKLPKYSLFELENGRKRMLASAGVLQKGNELALPSKY | |
| | VNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTINRKQYNTTKEV | |
| | LDATLIRQSITGLYETRIDLSQLGGDEGADKRTADGSEFESPKKKRKV | |
| 4071 | MSEVEFSHEYWMRHALTLAKRARDEREVPVGAVLVLNNRVIGEGWNRAIGLHDPT | 494 |
| | AHAEIMALRQGGLVMQNYRLYDATLYSTFEPCVMCAGAMIHSRIGRVVFGVRNAK | |
| | TGAAGSLMDVLHHPGMNHRVEITEGILADECAALLCRFFRMPRRVFNAQKKAQSS | |
| | TDSGGSSGGSSGSETPGTSESATPESSGGSSGGSDKKYSIGLAIGTNSVGWAVIT | |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFVSPTVAYSVLVVAKVEKGKSKKLKSVKELLGITIMERSSFEKN | |
| | PIDFLEAKGYKEVKKDLIIKLPKYSLFELENGRKRMLASARELQKGNELALPSKY | |
| | VNFLYLASHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKQYRSTKEV | |
| | LDATLIHQSITGLYETRIDLSQLGGDEGADKRTADGSEFESPKKKRKV | |
| 2626 | MKRTADGSEFESPKKKRKVSEVEFSHEYWMRHALTLAKRARDEREVPVGAVLVLN | 495 |
| | NRVIGEGWNRAIGLHDPTAHAEIMALRQGGLVMQNYRLYDATLYSTFEPCVMCAG | |
| | AMIHSRIGRVVFGVRNAKTGAAGSLMDVLHHPGMNHRVEITEGILADECAALLCR | |
| | FFRMPRRVFNAQKKAQSSTDSGGSSGGSSGSETPGTSESATPESSGGSSGGSDKK | |
| | YSIGLAIGTNSVGWAVITDEYKVPSKKFKVLGNTDRHSIKKNLIGALLFDSGETA | |
| | EATRLKRTARRRYTRRKNRICYLQEIFSNEMAKVDDSFFHRLEESFLVEEDKKHE | |
| | RHPIFGNIVDEVAYHEKYPTIYHLRKKLVDSTDKADLRLIYLALAHMIKFRGHFL | |
| | IEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMIKRYDEHHQDLTL | |
| | LKALVRQQLPEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKDNREKIEKIL | |
| | TFRIPYYVGPLARGNSRFAWMTRKSEETITPWNFEEVVDKGASAQSFIERMTNFD | |
| | KNLPNEKVLPKHSLLYEYFTVYNELTKVKYVTEGMRKPAFLSGEQKKAIVDLLFK | |
| | TNRKVTVKQLKEDYFKKIECFDSVEISGVEDRFNASLGTYHDLLKIIKDKDFLDN | |
| | EENEDILEDIVLTLTLFEDREMIEERLKTYAHLFDDKVMKQLKRRRYTGWGRLSR | |
| | KLINGIRDKQSGKTILDFLKSDGFANRNFMQLIHDDSLTFKEDIQKAQVSGQGDS | |
| | LHEHIANLAGSPAIKKGILQTVKVVDELVKVMGRHKPENIVIEMARENQTTQKGQ | |
| | KNSRERMKRIEEGIKELGSQILKEHPVENTQLQNEKLYLYYLQNGRDMYVDQELD | |
| | INRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRM | |
| | NTKYDENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGG | |
| | FSKESIRPKRNSDKLIARKKDWDPKKYGGFVSPTVAYSVLVVAKVEKGKSKKLKS | |
| | VKELLGITIMERSSFEKNPIDFLEAKGYKEVKKDLIIKLPKYSLFELENGRKRML | |
| | ASARFLQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPRAF | |
| | KYFDTTIDRKAYRSTKEVLDATLIHQSITGLYETRIDLSQLGGDEGADKRTADGS | |
| | EFESPKKKRKV | |
| 3626 | MKRTADGSEFESPKKKRKVSEVEFSHEYWMRHALTLAKRARDEREVPVGAVLVLN | 496 |
| | NRVIGEGWNRAIGLHDPTAHAEIMALRQGGLVMQNYRLYDATLYSTFEPCVMCAG | |
| | AMIHSRIGRVVFGVRNAKTGAAGSLMDVLHHPGMNHRVEITEGILADECAALLCR | |
| | FFRMPRRVFNAQKKAQSSTDSGGSSGGSSGSETPGTSESATPESSGGSSGGSDKK | |
| | YSIGLAIGTNSVGWAVITDEYKVPSKKFKVLGNTDRHSIKKNLIGALLFDSGETA | |
| | EATRLKRTARRRYTRRKNRICYLQEIFSNEMAKVDDSFFHRLEESFLVEEDKKHE | |
| | RHPIFGNIVDEVAYHEKYPTIYHLRKKLVDSTDKADLRLIYLALAHMIKFRGHFL | |
| | IEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLL | |
| | AQIGDQYADLFLAAKNLSDAILLSDILRVNTEITKAPLSASMVKRYDEHHQDLTL | |
| | LKALVRQQLPEKYKEIFFDQSKNGYAGYIDGGASQEEFYKFIKPILEKMDGTEEL | |
| | LVKLNREDLLRKQRTFDNGIIPHQIHLGELHAILRRQGDFYPFLKDNREKIEKIL | |
| | TFRIPYYVGPLARGNSRFAWMTRKSEETITPWNFEEVVDKGASAQSFIERMTNFD | |
| | KNLPNEKVLPKHSLLYEYFTVYNELTKVKYVTEGMRKPAFLSGEQKKAIVDLLFK | |
| | TNRKVTVKQLKEDYFKKIECFDSVEISGVEDRFNASLGTYHDLLKIIKDKDFLDN | |
| | EENEDILEDIVLTLTLFEDREMIEERLKTYAHLFDDKVMKQLKRLRYTGWGRLSR | |
| | KLINGIRDKQSGKTILDFLKSDGFANRNFMQLIHDDSLTFKEDIQKAQVSGQGDS | |
| | LHEHIANLAGSPAIKKGILQTVKVVDELVKVMGGHKPENIVIEMARENQTTQKGQ | |
| | KNSRERMKRIEEGIKELGSQILKEHPVENTQLQNEKLYLYYLQNGRDMYVDQELD | |
| | INRLSDYDVDHIVPQSFLKDDSIDNKVLTRSDKNRGKSDNVPSEEVVKKMKNYWR | |
| | QLLNAKLITQRKFDNLTKAERGGLSELDKAGFIKRQLVETRQITKHVAQILDSRM | |
| | NTKYDENDKLIREVKVITLKSKLVSDFRKDFQFYKVREINNYHHAHDAYLNAVVG | |
| | TALIKKYPKLESEFVYGDYKVYDVRKMIAKSEQEIGKATAKYFFYSNIMNFFKTE | |
| | ITLANGEIRKRPLIETNGETGEIVWDKGRDFATVRKVLSMPQVNIVKKTEVQTGG | |
| | FSKESILPKGNSDKLIARKKDWDPKKYGGFMQPTVAYSVLVVAKVEKGKSKKLKS | |
| | VKELLGITIMERSSFEKNPIDFLEAKGYKEVKKDLIIKLPKYSLFELENGRKRML | |
| | ASAKFLQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPRAF | |
| | KYFDTTIARKEYRSTKEVLDATLIHQSITGLYETRIDLSQLGGDEGADKRTADGS | |
| | EFESPKKKRKV | |
| 3167 | MKRTADGSEFESPKKKRKVDKKYSIGLAIGTNSVGWAVITDEYKVPSKKFKVLGN | 497 |
| | TDRHSIKKNLIGALLFDSGETAEATRLKRTARRRYTRRKNRICYLQEIFSNEMAK | |
| | VDDSFFHRLEESFLVEEDKKHERHPIFGNIVDEVAYHEKYPTIYHLRKKLVDSTD | |
| | KADLRLIYLALAHMIKFRGHFLIEGDLNPDNSDVDKLFIQLVQTYNQLFEENPIN | |
| | ASGVDAKAILSARLSKSRRLENLIAQLPGEKKNGLFGNLIALSLGLTPNFKSNFD | |
| | LAEDAKLQLSKDTYDDDLDNLLAQIGDQYADLFLAAKNLSDAILLSDILRVNTEI | |
| | TKAPLSASMVKRYDEHHQDLTLLKALVRQQLPEKYKEIFFDQSKNGYAGYIDGGA | |
| | SQEEFYKFIKPILEKMDGTEELLVKLNREDLLRKQRTFDNGIIPHQIHLGELHAI | |
| | LRRQGDFYPFLKDNREKIEKILTFRIPYYVGPLARGNSRFAWMTRKSEETITPWN | |
| | FEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAHL | |
| | FDDKVMKQLKRLRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMG | |
| | GHKPENIVIEMARENQTTQKGQKNSRERMKRIEEGIKELGSQILKEHPVENTQLQ | |
| | NEKLYLYYLQNGRDMYVDQELDINRLSDYDVDHIVPQSFLKDDSIDNKVLTRSDK | |
| | NRGKSDNVPSEEVVKKMKNYWRQLLNAKLITQRKFDNLTKAERGGLSELDKAGFI | |
| | KRQLVETRQITKHVAQILDSRMNTKYDENDKLIREVKVITLKSKLVSDFRKDFQF | |
| | YKVREINNYHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKGRDFAT | |
| | VRKVLSMPQVNIVKKTEVQTGGFSKESILPKGNSDKLIARKKDWDPKKYGGFMQP | |
| | TVAYSVLVVAKVEKGKSKKLKSVKELLGITIMERSSFEKNPIDFLEAKGYKEVKK | |
| | DLIIKLPKYSLFELENGRKRMLASAKFLQKGNELALPSKYVNFLYLASHYEKLKG | |
| | GSSGSETPGTSESATPESSGSEVEFSHEYWMRHALTLAKRARDEREVPVGAVLVL | |
| | NNRVIGEGWNRAIGLHDPTAHAEIMALRQGGLVMQNYRLYDATLYSTFEPCVMCA | |
| | GAMIHSRIGRVVFGVRNAKTGAAGSLMDVLHHPGMNHRVEITEGILADECAALLC | |
| | RFFRMPRRVFNAQKKAQSSTDSPEDNEQKQLFVEQHKHYLDEIIEQISEFSKRVI | |
| | LADANLDKVLSAYNKHRDKPIREQAENIIHLFTLTNLGAPRAFKYFDTTIARKEY | |
| | RSTKEVLDATLIHQSITGLYETRIDLSQLGGDEGADKRTADGSEFESPKKKRKV | |

### OTHER EMBODIMENTS

From the foregoing description, it will be apparent that variations and modifications may be made to the various embodiments of the disclosure described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. This application may be related to PCT/US2020/048586, filed Aug. 28, 2020, the disclosure of which is herein incorporated by reference in its entirety for all purposes.

What is claimed:

1. A method of producing an edited hematopoietic stem or progenitor cell (HSPC) for the treatment of a disease or condition, the method comprising:
(a) expressing in the hematopoietic stem cell or progenitor thereof a nucleobase editor polypeptide, wherein the nucleobase editor polypeptide comprises a nucleic acid programmable DNA binding protein (napDNAbp) domain and a deaminase domain; and
(b) contacting the hematopoietic stem cell or progenitor thereof with a guide RNA (gRNA), or a polynucleotide encoding the gRNA, wherein the gRNA targets a polynucleotide encoding a cluster of differentiation 45 (CD45) polypeptide, thereby introducing a missense mutation in a CD45 polynucleotide in the cell, wherein the missense mutation is in a portion of the polynucleotide encoding the extracellular domain and/or in fibronectin domain 1 to domain 4 of the CD45 polypeptide, and wherein the missense mutation is associated with a reduction in binding of an anti-CD45 antibody to the CD45 polypeptide expressed by the edited HSPC.

2. The method of claim 1, wherein the anti-CD45 antibody comprises the following CDRs: VH CDR1: GFDFSRYW (SEQ ID NO: 430); VH CDR2: INPTSSTI (SEQ ID NO: 431); VH CDR3: ARGNYYRYGDAMDY (SEQ ID NO: 432); VL CDR1: KSVSTSGYSYL (SEQ ID NO: 433); VL CDR2: LAS; and VL CDR3: QHSRELPFT (SEQ ID NO: 434).

3. The method of claim 1, wherein the deaminase domain comprises an adenosine deaminase domain or a cytidine deaminase domain.

4. The method of claim 1, wherein the adenosine deaminase domain comprises TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, TadA*8.24, TadA-8e, or TadA*8.20 with the amino acid alterations S82T, Y147D, F149Y, T166I, and D167N.

5. The method of claim 1, wherein the adenosine deaminase domain is inserted within the napDNAbp domain.

6. The method of claim 1, wherein the missense mutation is selected from the group consisting of
E256G;
D238G, Y239C;
E259G;
E259K;
E259G, N262D;
I283M;
I283M, H285R;
I283M, H285R, N286G;
K231R, Y232C;
N253G, N255G;
N255G, E256G;
N255G, E256G, N257D;
N255G, E256G, N257G;
N257G;
N257S;
N257D;
E259R;
N257G, E256G;
N257G, E256G, N257D;
N263G, T264A;
N263S, T264A;
N263S, T264A, T266A;
N267G, N268G;
N267S;
N267G;
N286G;
T264A;
T266A;
T266A, N267G; and
N286D.

7. A method of conditioning a human subject having a disease or condition concurrent with or prior to a hematopoietic stem cell transplant (HSCT), the method comprising:
(a) expressing in an hematopoietic stem cell of the subject or of a donor a nucleobase editor polypeptide comprising a nucleic acid programmable DNA binding protein (napDNAbp) and a deaminase;
(b) contacting the hematopoietic stem cell or progenitor cell (HSPC) with a guide RNA (gRNA), or a polynucleotide encoding the gRNA, wherein the gRNA targets a polynucleotide encoding a cluster of differentiation 45 (CD45) polypeptide, thereby introducing a missense mutation in the polynucleotide and generating an edited HSPC, wherein the missense mutation is in a portion of the polynucleotide encoding the extracellular domain and/or in fibronectin domain 1 to domain 4 of the CD45 polypeptide;
(c) administering the edited hematopoietic stem cell to the subject; and
(d) administering to the subject an anti-CD45 antibody, anti-CD45 antibody drug conjugate, or anti-CD45 chimeric antigen receptor expressing T cell (CAR-T) that selectively binds a wild-type CD45 protein, wherein the administering of step (d) is prior to, concurrent with, or following step (c),
wherein the missense mutation is associated with a reduction in binding of the anti-CD45 antibody, anti-CD45 antibody drug conjugate, or anti-CD45 chimeric antigen receptor expressing T cell (CAR-T) to a CD45 polypeptide expressed by the edited HSPC.

8. The method of claim 7, wherein the anti-CD45 antibody, anti-CD45 antibody drug conjugate, or anti-CD45 chimeric antigen receptor comprises the following CDRs: VH CDR1: GFDFSRYW (SEQ ID NO: 430); VH CDR2: INPTSSTI (SEQ ID NO: 431); VH CDR3: ARGNYYRYGDAMDY (SEQ ID NO: 432); VL CDR1: KSVSTSGYSYL (SEQ ID NO: 433); VL CDR2: LAS; and VL CDR3: QHSRELPFT (SEQ ID NO: 434).

9. A base editor system comprising a nucleobase editor polypeptide, or a polynucleotide encoding the nucleobase editor polypeptide, wherein the nucleobase editor polypeptide comprises a nucleic acid programmable DNA binding protein (napDNAbp) domain and a deaminase domain, and a guide RNA (gRNA), or a polynucleotide encoding the gRNA, wherein the gRNA targets a polynucleotide encoding a cluster of differentiation 45 (CD45) polypeptide, thereby introducing a missense mutation in a CD45 polynucleotide, wherein the missense mutation is in a portion of the polynucleotide encoding the extracellular domain and/or in fibronectin domain 1 to domain 4 of the CD45 polypeptide, and wherein the missense mutation is associated with a reduction in binding of an anti-CD45 antibody to the CD45 polypeptide expressed by the edited HSPC.

10. The base editor system of claim 9, wherein the deaminase domain comprises an adenosine deaminase domain or a cytidine deaminase domain.

11. The base editor system of claim 10, wherein the adenosine deaminase domain comprises TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, TadA*8.24, TadA-8e, or TadA*8.20 with the amino acid alterations S82T, Y147D, F149Y, T166I, and D167N.

12. The base editor system of claim 11, wherein the adenosine deaminase domain comprises TadA*8.20, TadA-8e, or TadA*8.20 with the amino acid alterations S82T, Y147D, F149Y, T166I, and D167N.

13. A polynucleotide or set of polynucleotides encoding the base editor system of claim 9, or a component thereof.

14. A vector or set of vectors comprising the polynucleotide of claim 10.

15. An HSPC prepared by the method claim 1.

16. An HSPC comprising the base editor system of claim 9.

17. A pharmaceutical composition comprising the HSPC of claim 13, and a pharmaceutically acceptable excipient.

18. A kit suitable for use in the method of claim 1.

19. A method of producing an edited hematopoietic stem or progenitor cell (HSPC) for the treatment of a disease or condition, the method comprising:
(a) expressing in the hematopoietic stem cell or progenitor thereof a nucleobase editor polypeptide, wherein the nucleobase editor polypeptide comprises an SpCas9 polypeptide having specificity for a protospacer adjacent motif selected from NRCH and NGC, wherein “N” is A, T, G, or C, “R” is A or G, and “H” is A, C, or T, and an adenosine deaminase domain, wherein the adenosine deaminase domain comprises TadA*8.20, TadA-8e, or TadA*8.20 with the amino acid alterations S82T, Y147D, F149Y, T166I, and D167N; and
(b) contacting the hematopoietic stem cell or progenitor thereof with gRNA4630 or gRNA2696, thereby introducing a missense mutation in a CD45 polynucleotide in the cell, wherein the missense mutation results in an E259G alteration to the CD45 polypeptide encoded by the CD45 polynucleotide.

20. A method of treating a subject having a disease or condition, the method comprising:
a) administering to the subject the HSPC cell of claim 19; and
b) administering to the subject an mAb030-9 polypeptide prior to, concurrent with, or following step a).
